(19) United States
(12) Patent Application Publication
(10) Pub. No.: US 2026/0325299 A1
(43) Pub. Date: Oct. 22, 2026

(54) DELIVERY SYSTEM FOR MEDICAL IMPLANT

(71) Applicant: Edwards Lifesciences Corporation, Irvine, CA (US)

(72) Inventors: David Robert Landon, Huntington Beach, CA (US); Alexander H. Cooper, Costa Mesa, CA (US); Glen T. Rabito, Lake Forest, CA (US); Julio Cesar Sanchez, Garden Grove, CA (US); Siddharth Vad, Irvine, CA (US)

(21) Appl. No.: 19/724,587

(22) Filed: Jun. 29, 2026

Related U.S. Application Data
(63) Continuation of application No. 17/581,724, filed on Jan. 21, 2022.
Application No. 17/581,724 is a continuation of application No. PCT/US2020/040329, filed on Jun. 30, 2020.
(60) Provisional application No. 62/879,986, filed on Jul. 29, 2019.
(60) Provisional application No. 62/975,587, filed on Feb. 12, 2020.

(51) Int. Cl. A61F 2/24; A61M 25/00; A61M 25/01

(52) U.S. Cl. CPC A61F 2/2436; A61F 2/2418; A61F 2250/0018; A61F 2250/0029; A61M 2025/0004; A61M 25/0045; A61M 25/005; A61M 25/0108; A61M 25/0138

(57) ABSTRACT

Embodiments of the present disclosure are directed to delivery systems, devices and/or methods of use to an implant to a location within a patient's body. The delivery system includes a shaft. The shaft includes a retention member. The retention member is configured to retain the implant. The retention member is positioned at a distal end portion of the shaft. The delivery system includes a sheath. The sheath is slidably mounted on the shaft. The sheath is configured to surround the retention member. The delivery system includes a stop. The stop is positioned at the distal end portion of the shaft. The stop is positioned proximal to the retention member. The stop is configured to define a stopping point at which the stop impedes proximal sliding of the sheath past the stop.

### CROSS REFERENCE TO RELATED APPLICATIONS

This application is a continuation of U.S. patent application Ser. No. 17/581,724 to Scheinblum et al., filed Jan. 21, 2022, which is a continuation of International Patent Application No. PCT/US2020/040329, filed Jun. 30, 2020, which designates the United States and was published in English by the International Bureau on Feb. 4, 2021 as WO 2021/021368, which claims priority to U.S. Provisional Patent App. No. 62/879,986, filed Jul. 29, 2019, and U.S. Provisional Patent App. No. 62/975,587, filed Feb. 12, 2020, the entirety of each of these applications being incorporated herein by reference.

### FIELD

Certain embodiments disclosed herein relate generally to delivery systems for implants. In particular, delivery systems and implants relate in some embodiments to replacement heart valves, such as replacement mitral heart valves or other heart valves.

### BACKGROUND

Human heart valves, which include the aortic, pulmonary, mitral and tricuspid valves, function essentially as one-way valves operating in synchronization with the pumping heart. The valves allow blood to flow downstream, but block blood from flowing upstream. Diseased heart valves exhibit impairments such as narrowing of the valve or regurgitation, which inhibit the valves'ability to control blood flow. Such impairments reduce the heart's blood-pumping efficiency and can be a debilitating and life-threatening condition. For example, valve insufficiency can lead to conditions such as heart hypertrophy and dilation of the ventricle. Thus, extensive efforts have been made to develop methods and apparatuses to repair or replace impaired heart valves.

Prosthetic implants exist to correct problems associated with impaired heart valves. For example, mechanical and tissue-based heart valve prostheses can be used to replace impaired native heart valves. More recently, substantial effort has been dedicated to developing replacement heart valves, particularly tissue-based replacement heart valves that can be delivered with less trauma to the patient than through open heart surgery. Replacement valves are being designed to be delivered through minimally invasive procedures and even percutaneous procedures. Such replacement valves often include a tissue-based valve body that is connected to an expandable frame that is then delivered to the native valve's annulus.

Development of prosthetic implants including but not limited to replacement heart valves that can be compacted for delivery and then controllably expanded for controlled placement has proven to be particularly challenging. An additional challenge relates to the ability of such prostheses to be secured relative to intralumenal tissue, e.g., tissue within any body lumen or cavity, in an atraumatic manner.

Delivering an implant to a desired location in the human body, for example delivering a replacement heart valve to the mitral valve, can also be challenging. Obtaining access to perform procedures in the heart or in other anatomical locations may require delivery of devices percutaneously through tortuous vasculature or through open or semi-open surgical procedures. The ability to control the deployment of the prosthesis at the desired location can also be challenging.

It may also be difficult to properly visualize or locate a delivery system for the implant, including utilizing ultrasound imaging.

### SUMMARY

Embodiments of the present disclosure are directed to delivery systems, devices and/or methods of use to deliver and/or controllably deploy an implant in the form of a prosthesis, such as but not limited to a replacement heart valve, to a desired location within the body. In some embodiments, a replacement heart valve and methods for delivering a replacement heart valve to a native heart valve, such as a mitral valve, are provided.

In some embodiments, a delivery system and method are provided for delivering a replacement heart valve to a native mitral valve location. The delivery system and method may utilize a transseptal approach. In some embodiments, components of the delivery system facilitate bending of the delivery system to steer a prosthesis from the septum to a location within the native mitral valve. In some embodiments, a capsule is provided for containing the prosthesis for delivery to the native mitral valve location. In other embodiments, the delivery system and method may be adapted for delivery of implants to locations other than the native mitral valve.

An embodiment herein includes a delivery system for delivering an implant to a location within a patient's body, the delivery system comprising an elongate shaft having a proximal end and a distal end, and including: an implant retention area configured to retain the implant, and a capsule configured to surround the implant retention area and including a hypotube having one or more cuts forming a plurality of rings.

An embodiment herein includes a delivery system for delivering an implant to a location within a patient's body, the delivery system comprising: an elongate shaft having a proximal end and a distal end, and including: an implant retention area configured to retain the implant, and a capsule configured to surround the implant retention area and including a hypotube having one or more cuts that bias a flexibility of the hypotube in a direction.

An embodiment herein includes a delivery system for delivering an implant to a location within a patient's body, the delivery system comprising: an elongate shaft having a proximal end and a distal end, and including: an implant retention area configured to retain the implant, and a capsule configured to surround the implant retention area and including a hypotube having one or more cuts forming a spiral.

An embodiment herein includes a method comprising: deploying an elongate shaft to a location within a patient's body, the elongate shaft including a capsule surrounding an implant retention area retaining an implant for implantation within the patient's body, the capsule including a hypotube having one or more cuts forming a plurality of rings; moving the capsule proximally to expose a portion of the implant within the patient's body; and moving the capsule distally to recapture a portion of the implant within the patient's body.

An embodiment herein includes a delivery system for delivering an implant to a location within a patient's body, the delivery system comprising: an elongate shaft having a proximal end and a distal end, and including: an implant retention area configured to retain the implant, a capsule configured to surround the implant retention area, a shaft portion positioned proximal of the capsule, and a coupler configured to couple the capsule to the shaft portion and configured to allow the capsule to rotate relative to the shaft portion.

An embodiment herein includes a method comprising: deploying an elongate shaft to a location within a patient's body, the elongate shaft including a capsule and a shaft portion positioned proximal of the capsule and a coupler coupling the capsule to the shaft portion and configured to allow the capsule to rotate relative to the shaft portion, the capsule surrounding an implant retention area retaining an implant for implantation within the patient's body; and moving the capsule proximally to expose a portion of the implant within the patient's body while the capsule rotates relative to the shaft portion.

An embodiment herein includes a delivery system for delivering an implant to a location within a patient's body, the delivery system comprising: an elongate shaft having a proximal end and a distal end, and including: an implant retention area configured to retain the implant, a sheath having an interior lumen, an interior shaft positioned within the interior lumen, and an inflatable body positioned between the interior shaft and the sheath and configured to inflate to support the sheath.

An embodiment herein includes a method comprising: deploying an elongate shaft to a location within a patient's body, the elongate shaft including a sheath and an interior shaft positioned within an interior lumen of the sheath and an implant retention area retaining an implant for implantation within the patient's body; moving the sheath proximally to expose a portion of the implant within the patient's body; inflating an inflatable body between the sheath and the interior shaft; and moving the sheath distally to recapture a portion of the implant within the patient's body while the inflatable body is inflated between the sheath and the interior shaft to support the sheath.

An embodiment herein includes a delivery system for delivering an implant to a location within a patient's body, the delivery system comprising: an elongate shaft having a proximal end and a distal end, and including: an implant retention area configured to retain the implant, and a wall of the elongate shaft including: an outer jacket layer, an interior liner layer, a braid layer positioned between the outer jacket layer and the interior liner layer, and a metal layer positioned between the braid layer and the interior liner layer.

An embodiment herein includes a delivery system for delivering an implant to a location within a patient's body, the delivery system comprising: an elongate shaft having a proximal end and a distal end, and including: an implant retention area configured to retain the implant, a sheath configured to bend in at least one plane, and a cable having a first end portion, a second end portion, and an intermediate portion extending between the first end portion and the second end portion, the first end portion coupled to a first side of the sheath and the second end portion coupled to a second side of the sheath opposite the first side. The delivery system includes a cable router configured to engage the intermediate portion of the cable and allow the cable to move along the cable router when the sheath is bent in the at least one plane; and a control mechanism for retracting the cable router and the sheath relative to the implant retention area.

An embodiment herein includes a method comprising: deploying an elongate shaft to a location within a patient's body, the elongate shaft including an implant retention area retaining an implant for implantation within the patient's body and a sheath and a cable having a first end portion, a second end portion, and an intermediate portion extending between the first end portion and the second end portion, the first end portion coupled to a first side of the sheath and the second end portion coupled to a second side of the sheath opposite the first side, the intermediate portion engaged with a cable router; bending the sheath in a plane such that a length of the cable between the cable router and the first end portion increases and a length of the cable between the cable router and the second end portion decreases; and retracting the cable router and the sheath relative to the implant retention area.

An embodiment herein includes a delivery system for delivering an implant to a location within a patient's body, the delivery system comprising: an elongate shaft having a proximal end and a distal end, and including: an implant retention area configured to retain the implant, a sheath having an interior lumen and a capsule configured to surround the implant retention area, an interior shaft positioned within the interior lumen, and a stop positioned on the interior shaft and configured to impede proximal movement of the capsule relative to the interior shaft.

An embodiment herein includes a method comprising: deploying an elongate shaft to a location within a patient's body, the elongate shaft including a sheath having an interior lumen and a capsule retaining an implant for implantation within the patient's body, the sheath including an interior shaft and a stop positioned on the interior shaft; moving the capsule proximally to expose a first portion of the implant within the patient's body until the sheath contacts the stop; and overcoming the stop and moving the capsule proximally to expose a second portion of the implant within the patient's body that is positioned proximal of the first portion of the implant.

An embodiment herein includes a delivery system for delivering an implant to a location within a patient's body, the delivery system comprising: an elongate shaft having a proximal end and a distal end, and an implant retention area, and an assembly configured to retain at least a portion of the implant within the implant retention area; and a handle coupled to the proximal end of the elongate shaft and including a control knob configured to be rotated to move the assembly to release at least the portion of the implant from the implant retention area, the control knob including an outer grip surface that is exposed for gripping around an entire outer circumference of the control knob.

An embodiment herein includes a method comprising: deploying a delivery apparatus to a location within a patient's body, the delivery apparatus including an elongate shaft and a handle coupled to a proximal end of the elongate shaft, the elongate shaft including an implant retention area retaining an implant for implantation within the patient's body and an assembly configured to retain at least a portion of the implant within the implant retention area, the handle including a control knob configured to be rotated to move the assembly to release at least the portion of the implant from the implant retention area and including an outer grip surface that is exposed for gripping around an entire outer circumference of the control knob; gripping the grip surface of the control knob; and rotating the control knob to move the assembly to release at least the portion of the implant from the implant retention area.

An embodiment herein includes a delivery system for delivering an implant to a location within a patient's body, the delivery system comprising: an elongate shaft having a proximal end and a distal end, and including: an implant retention area configured to retain the implant, and a marker configured to enhance an echogenicity of the elongate shaft to define a location of a portion of the elongate shaft when viewed with ultrasound imaging.

An embodiment herein includes a method comprising: deploying an elongate shaft to a location within a patient's body, the elongate shaft including an implant retention area retaining an implant for implantation within the patient's body and a marker that enhances an echogenicity of the elongate shaft to define a location of a portion of the elongate shaft when viewed with ultrasound imaging.

An embodiment herein includes a delivery system for delivering an implant to a location within a patient's body, the delivery system comprising: an elongate shaft having a proximal end and a distal end, and including: an implant retention area configured to retain the implant, and a capsule configured to surround the implant retention area and including a distal end configured to expand radially outward.

An embodiment herein includes a method comprising: deploying an elongate shaft to a location within a patient's body, the elongate shaft including a capsule surrounding an implant retention area retaining an implant for implantation within the patient's body; moving the capsule proximally to expose a portion of the implant within the patient's body; and moving the capsule distally to recapture a portion of the implant within the patient's body and to pass a distal end of the capsule that is expanded radially outward over the portion of the implant that is recaptured.

An embodiment herein includes a delivery system for delivering an implant to a location within a patient's body, the delivery system comprising: an elongate shaft having a proximal end and a distal end, and including: an implant retention area configured to retain the implant, and a pull tether coupled to a portion of the elongate shaft at or distal the implant retention area and configured to deflect the distal end of the elongate shaft.

An embodiment herein includes a delivery system for delivering an implant to a location within a patient's body, the delivery system comprising: an elongate shaft having a proximal end and a distal end, and including: an implant retention area configured to retain the implant, a nose cone positioned at the distal end of the elongate shaft, and a pull tether coupled to the nose cone and configured to deflect the nose cone.

An embodiment herein includes a method comprising: deploying an elongate shaft to a location within a patient's body, the elongate shaft including a proximal end and a distal end and an implant retention area retaining an implant for implantation within the patient's body; and deflecting the distal end of the elongate shaft utilizing a pull tether coupled to a portion of the elongate shaft at or distal the implant retention area.

An embodiment herein includes a delivery system for delivering an implant to a location within a patient's body, the delivery system comprising: a first elongate shaft having a proximal end and a distal end and extending along a first axis and being steerable; a second elongate shaft having a proximal end and a distal end and extending along a second axis and including an implant retention area configured to retain the implant; and a coupler configured to couple the second elongate shaft to the first elongate shaft such that the second elongate shaft may slide relative to the first elongate shaft with the second axis offset from the first axis.

An embodiment herein includes a method comprising: deploying a first elongate shaft to a location within a patient's body, the first elongate shaft extending along a first axis and being steerable; and sliding a second elongate shaft along the first elongate shaft to a location within the patient's body, the second elongate shaft being coupled to the first elongate shaft and extending along a second axis that is offset from the first axis and including an implant retention area retaining an implant therein.

An embodiment herein includes a delivery system for delivering an implant to a location within a patient's body, the delivery system comprising: an elongate shaft having a proximal end and a distal end, and including an implant retention area configured to retain the implant, and a covering layer on the elongate shaft including reinforcing fibers or beads.

An embodiment herein includes a method comprising: deploying an elongate shaft to a location within a patient's body, the elongate shaft including an implant retention area retaining an implant for implantation within the patient's body and a covering layer including reinforcing fibers or beads.

An embodiment herein includes a method comprising: preparing a mixture of polytetrafluoroethylene (PTFE) with reinforcing fibers or beads; and providing an elongate shaft for a delivery system for delivering an implant to a location within a patient's body, the elongate shaft including the mixture of the polytetrafluoroethylene (PTFE) with the reinforcing fibers or beads as a covering layer of the elongate shaft.

### BRIEF DESCRIPTION OF THE DRAWINGS

FIG. 1 shows an embodiment of a delivery system.

FIG. 2A shows a partial cross-sectional view of the distal end of the delivery system of FIG. 1 loaded with the valve prosthesis of FIG. 3A.

FIG. 2B shows a partial cross-sectional view of the distal end of the delivery system of FIG. 1 without the valve prosthesis of FIG. 3A.

FIG. 2C shows a partial cross-sectional view of the distal end of the delivery system of FIG. 1 without with certain shaft assemblies translated along the rail assembly.

FIG. 3A shows a side view of an embodiment of a valve prosthesis that may be delivered using the delivery systems described herein.

FIG. 3B shows a side view of an embodiment of an aortic valve prosthesis that may be delivered using the delivery systems described herein.

FIG. 4 shows a perspective view of the distal end of the delivery system of FIG. 1.

FIG. 5 shows components of the delivery system of FIG. 4 with the outer sheath assembly moved proximally and out of view.

FIG. 6A shows components of the delivery system of FIG. 5 with the mid shaft assembly moved proximally and out of view.

FIG. 6B illustrates a cross-section view of the rail assembly.

FIG. 7 shows components of the delivery system of FIG. 6A with the rail assembly moved proximally and out of view.

FIG. 8 shows components of the delivery system of FIG. 7 with the inner assembly moved proximally and out of view.

FIGS. 9A and 9B illustrate embodiments of a guide wire shield.

FIG. 10 illustrates an embodiment of an outer hypotube.

FIG. 11 illustrates an embodiment of a mid shaft hypotube.

FIG. 12A illustrates an embodiment of the mid shaft hypotube of FIG. 11 as a flat pattern.

FIG. 12B illustrates an embodiment of an outer retention ring.

FIG. 13 illustrates an embodiment of a rail assembly.

FIG. 14 illustrates an embodiment of an inner assembly.

FIG. 15 illustrates a cross-section of a capsule.

FIG. 16 illustrates an embodiment of a hypotube of an outer sheath assembly as a flat pattern.

FIG. 17 illustrates an embodiment of a hypotube of an outer sheath assembly as a flat pattern.

FIG. 18 illustrates an embodiment of a hypotube of an outer sheath assembly as a flat pattern.

FIG. 19 illustrates an embodiment of a hypotube of an outer sheath assembly as a flat pattern.

FIG. 20 illustrates an embodiment of a hypotube of an outer sheath assembly as a flat pattern.

FIG. 21 illustrates an embodiment of a hypotube of an outer sheath assembly as a flat pattern.

FIG. 22 illustrates a cross sectional view of a distal portion of an elongate shaft of a delivery system including a coupler coupling a capsule to a shaft.

FIG. 23 illustrates a perspective view of a distal portion of an elongate shaft of a delivery system including a coupler coupling a capsule to a shaft.

FIG. 24 illustrates a cross sectional view of a distal portion of an elongate shaft of a delivery system including a coupler coupling a capsule to a shaft.

FIG. 25 illustrates a perspective view of a distal portion of an elongate shaft of a delivery system including a coupler coupling a capsule to a shaft.

FIG. 26 illustrates a cross sectional view of a distal portion of an elongate shaft of a delivery system including an inflatable body positioned between an outer sheath and an interior shaft.

FIG. 27 illustrates a cross sectional view of the distal portion of the elongate shaft shown in FIG. 26 with the distal portion bent and the inflatable body inflated.

FIG. 28 illustrates a side view of an embodiment of a braid layer.

FIG. 29 illustrates a cross sectional schematic of a construction of a wall of an elongate shaft.

FIG. 30 illustrates a cross sectional schematic of components of an elongate shaft including a sheath, a cable, and a cable router.

FIG. 31 illustrates a cross sectional schematic of the sheath shown in FIG. 30 bent.

FIGS. 32A, 32B, and 32C each illustrate a cross sectional representation of a stop for a capsule.

FIGS. 33A, 33B, and 33C each illustrate a cross sectional representation of a stop for a capsule.

FIGS. 34A, 34B, and 34C each illustrate a perspective representation view of a stop for a capsule.

FIG. 35 illustrates an embodiment of a delivery system handle.

FIG. 36 illustrates a cross-section of the delivery system handle of FIG. 35.

FIG. 37 illustrates a perspective view of an embodiment of a delivery system handle.

FIG. 38 illustrates a side bottom view of the delivery system handle of FIG. 37.

FIG. 39 illustrates a center cross sectional view of the delivery system handle of FIG. 37 from the side bottom view of FIG. 38.

FIG. 40 illustrates a front perspective view of the delivery system handle of FIG. 37.

FIG. 41 illustrates a perspective cross sectional view of the delivery system handle of FIG. 37 along line A-A in FIG. 39.

FIG. 42 illustrates a perspective cross sectional view of the delivery system handle of FIG. 37 along line B-B in FIG. 39.

FIG. 43 illustrates a perspective cross sectional view of the delivery system handle of FIG. 37 along line C-C in FIG. 39.

FIG. 44 illustrates a perspective cross sectional view of the delivery system handle of FIG. 37 along line D-D in FIG. 39.

FIG. 45 illustrates a perspective cross sectional view of the delivery system handle of FIG. 37 along line E-E in FIG. 39.

FIG. 46 illustrates a side view of an embodiment of a nose cone.

FIG. 47 illustrates a cross sectional view of the nose cone of FIG. 46 along line A-A in FIG. 46.

FIG. 48 illustrates a side view of an embodiment of a nose cone including a marker.

FIG. 49 illustrates views of echocardiogram images.

FIG. 50 illustrates a side view of an embodiment of a nose cone including a marker.

FIG. 51 illustrates a perspective view of an embodiment of a nose cone including a marker.

FIG. 52 illustrates a perspective view of an embodiment of a nose cone including a marker.

FIG. 53 illustrates a perspective view of an embodiment of a nose cone including a marker.

FIG. 54 illustrates a side cross sectional view of an embodiment of a nose cone including a marker.

FIG. 55 illustrates a perspective view of an embodiment of a nose cone with portions shown in transparent view and including a marker.

FIG. 56 illustrates a perspective view of the nose cone of FIG. 55.

FIG. 57 illustrates a perspective view of an embodiment of a nose cone including a marker.

FIG. 58 illustrates a cross sectional view of an embodiment of an outer sheath including a marker.

FIG. 59 illustrates a cross sectional view of the marker of FIG. 58.

FIG. 60 illustrates views of echocardiogram images.

FIG. 61 illustrates a schematic representation of a transseptal delivery approach.

FIG. 62 illustrates a schematic representation of a valve prosthesis positioned within a native mitral valve.

FIG. 63 shows a valve prosthesis frame located within a heart.

FIGS. 64-66 show steps of a method for delivery of the valve prosthesis to an anatomical location.

FIGS. 67A-B illustrate the methodology of the rail delivery system.

FIG. 68 shows a side view of an embodiment of an implant in the form of a valve prosthesis that may be delivered using the delivery systems described herein.

FIG. 69 show a view of an embodiment of an implant in the form of a valve prosthesis that may be delivered using the delivery systems described herein.

FIG. 70 shows a cross sectional view of arms of an implant extending from a capsule.

FIG. 71 shows a side cross sectional view of a distal end of a capsule expanding radially outward.

FIG. 72 shows a side cross sectional view of a capsule having a distal end configured to expand radially outward.

FIG. 73 shows a side cross sectional view of the capsule shown in FIG. 72.

FIG. 74A shows a side view of a capsule having a pull tether coupled to a nose cone.

FIG. 74B shows a side view of the capsule shown in FIG. 74A with the capsule flexed from the position shown in FIG. 74A.

FIG. 75A shows a side view of a capsule and nose cone of a delivery system.

FIG. 75B shows a side view of the capsule and nose cone of the delivery system shown in FIG. 75A.

FIG. 76A shows a perspective view of a capsule of a delivery system.

FIG. 76B shows a perspective view of the capsule of the delivery system shown in FIG. 76A.

FIG. 77A shows a perspective view of a capsule of a delivery system.

FIG. 77B shows a perspective view of the capsule of the delivery system shown in FIG. 77A.

FIG. 78A shows a perspective view of a capsule of a delivery system.

FIG. 78B shows a perspective view of the capsule of the delivery system shown in FIG. 78A.

FIG. 79 shows a side view of a steerable elongate shaft.

FIG. 80 shows a side view of an elongate shaft including an implant sliding along the steerable elongate shaft shown in FIG. 79.

FIG. 81 shows a side view of an elongate shaft including an implant sliding along the steerable elongate shaft shown in FIG. 79.

### DETAILED DESCRIPTION

The present specification and drawings provide aspects and features of the disclosure in the context of several embodiments of delivery systems and methods. The delivery systems and methods may be configured for use in the vasculature of a patient, such as for replacement of natural heart valves in a patient. These embodiments may be discussed in connection with replacing specific valves such as the patient's aortic, tricuspid, or mitral valve. However, it is to be understood that the features and concepts discussed herein can be applied to products other than heart valve implants. For example, the delivery systems and methods can be applied to medical implants, for example other types of expandable prostheses, for use elsewhere in the body, such as within an artery, a vein, or other body cavities or locations. In addition, particular features of a valve, delivery system, etc. should not be taken as limiting, and features of any one embodiment discussed herein can be combined with features of other embodiments as desired and when appropriate. While certain of the embodiments described herein are described in connection with a transfemoral delivery approach, it should be understood that these embodiments can be used for other delivery approaches such as, for example, transapical or transjugular approaches. Moreover, it should be understood that certain of the features described in connection with some embodiments can be incorporated with other embodiments, including those which are described in connection with different delivery approaches.

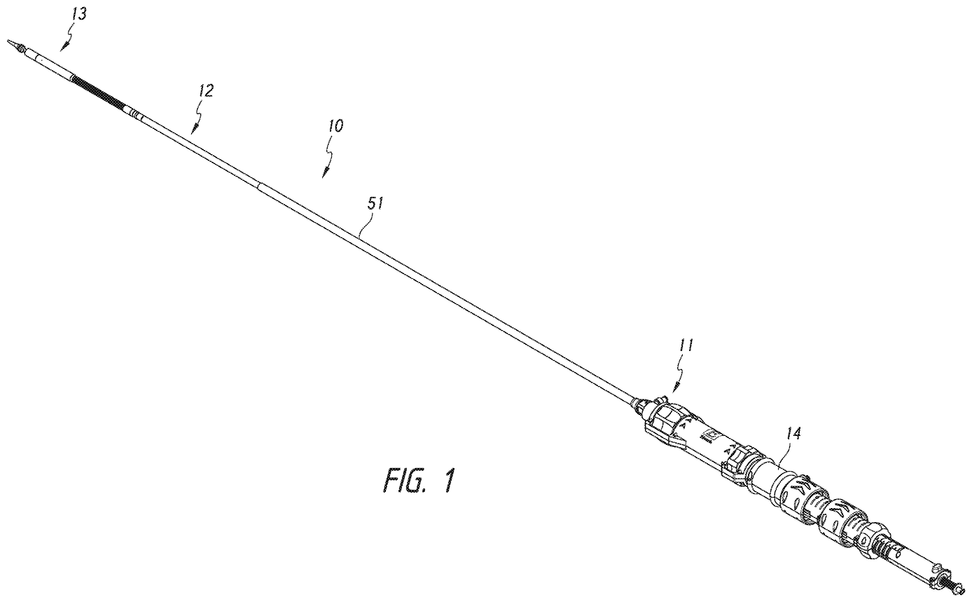

FIG. 1 illustrates an embodiment of a delivery system 10 according to an embodiment of the present disclosure. The delivery system 10 may be used to deploy an implant, such as a prosthetic replacement heart valve, within the body. In some embodiments, the delivery system 10 may use a dual plane deflection approach to properly deliver the implant. Replacement heart valves may be delivered to a patient's heart mitral valve annulus or other heart valve location in various manners, such as by open surgery, minimally-invasive surgery, and percutaneous or transcatheter delivery through the patient's vasculature. While the delivery system 10 may be described in certain embodiments in connection with a percutaneous delivery approach, and more specifically a transfemoral delivery approach, it should be understood that features of delivery system 10 can be applied to other delivery systems, including delivery systems for a transapical delivery approach.

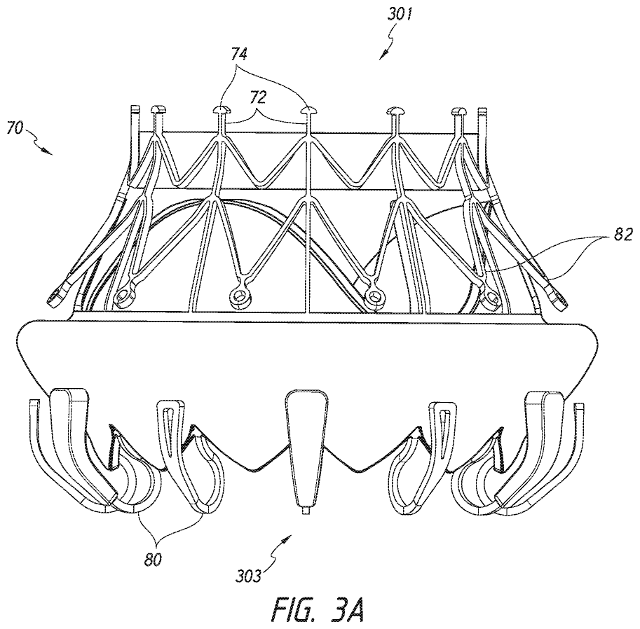

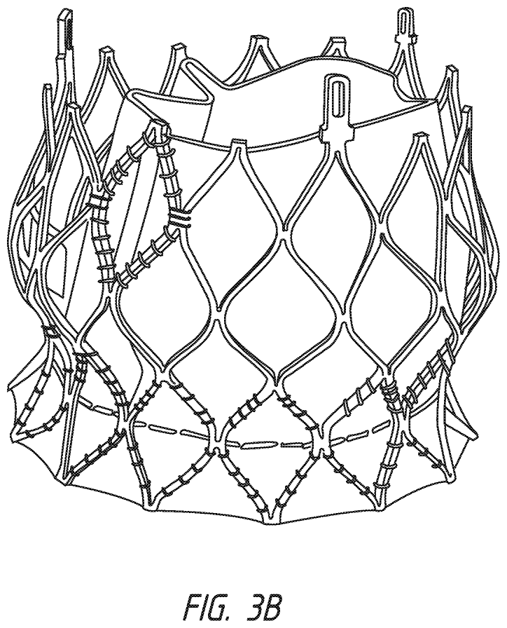

The delivery system 10 may be used to deploy an implant, such as a replacement heart valve as described elsewhere in this specification, within the body. The delivery system 10 may receive and/or cover portions of the implant such as a first end 301 and second end 303 of the implant 70, or prosthesis, illustrated in FIG. 3A. For example, the delivery system 10 may be used to deliver an expandable implant 70, where the implant 70 includes the first end 301 and the second end 303, and wherein the second end 303 is configured to be deployed or expanded before the first end 301.

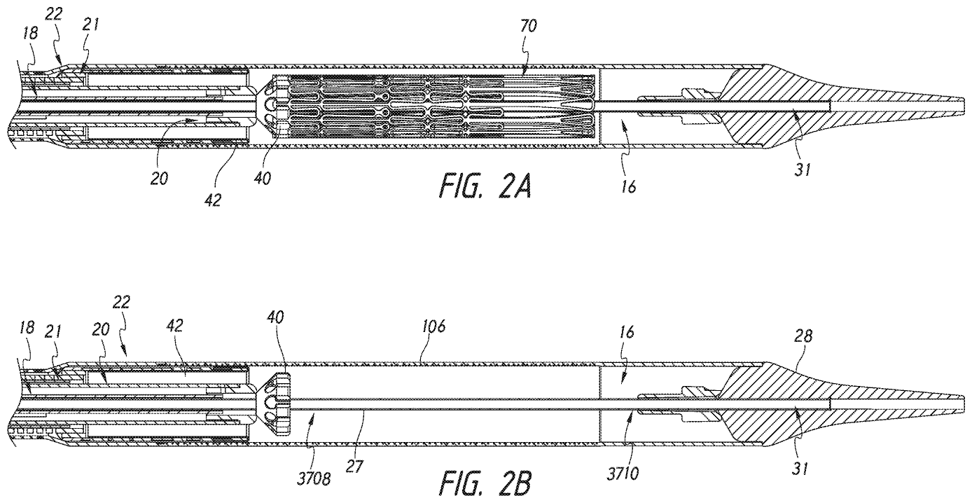

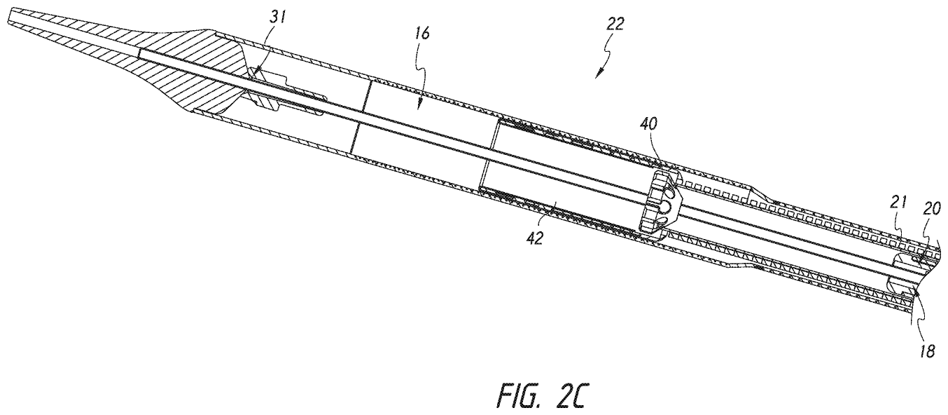

FIG. 2A further shows an example of the implant 70 that can be inserted into a portion of the delivery system 10, specifically into an implant retention area 16. For ease of understanding, in FIG. 2A, the implant is shown with only the bare metal frame illustrated. The implant 70, or prosthesis, can take any number of different forms. A particular example of frame for an implant is shown in FIG. 3A, although other designs may be utilized in other embodiments. The implant 70 may include one or more sets of anchors, such as distal (or ventricular) anchors 80 (marked in FIG. 3A) extending proximally when the implant frame is in an expanded configuration and proximal (or atrial) anchors 82 extending distally when the implant frame is in an expanded configuration. The implant may further include struts 72 which may end in mushroom-shaped tabs 74 at the first end 301 (marked in FIG. 3A).

In some embodiments, the delivery system 10 may be used in conjunction with a replacement aortic valve, such as shown in FIG. 3B. In some embodiments the delivery system 10 can be modified to support and deliver the replacement aortic valve. However, the procedures and structures discussed below can similarly be used for a replacement mitral and replacement aortic valve, as well as other replacement heart valves and other implants.

Referring to FIG. 1, the delivery system 10 may include an elongate shaft 12 that may comprise a shaft assembly. The elongate shaft 12 may include a proximal end 11 and a distal end 13, wherein a housing in the form of a handle 14 is coupled to the proximal end of the elongate shaft 12. The elongate shaft 12 may be used to hold the implant for advancement of the same through the vasculature to a treatment location. The elongate shaft 12 may further comprise a relatively rigid live-on (or integrated) sheath 51 surrounding an interior portion of the shaft 12 that may reduce unwanted motion of the interior portion of the shaft 12. The live-on sheath 51 can be attached at a proximal end of the shaft 12 proximal to the handle 14, for example at a sheath hub.

The elongate shaft 12 and housing in the form of a handle 14 may form a delivery apparatus that is configured to deliver the implant 70 to a body location.

Referring to FIGS. 2A and 2B, the elongate shaft 12 may include an implant retention area 16 (shown in FIGS. 2A-B with FIG. 2A showing the implant 70 and FIG. 2B with the implant 70 removed) at its distal end. In some embodiments, the elongate shaft 12 can hold an expandable implant in a compressed state at implant retention area 16 for advancement of the implant 70 within the body. The shaft 12 may then be used to allow controlled expansion of the implant 70 at the treatment location. In some embodiments, the shaft 12 may be used to allow for sequential controlled expansion of the implant 70 as discussed in detail below. The implant retention area 16 is shown in FIGS. 2A-2B at the distal end of the delivery system 10, but may also be at other locations. In some embodiments, the implant 70 may be rotated in the implant retention area 16, such as through the rotation of the inner shaft assembly 18 discussed herein.

As shown in the cross-sectional view of FIGS. 2A-2B, the distal end of the delivery system 10 can include one or more assemblies such as an outer sheath assembly 22, a mid shaft assembly 21, a rail assembly 20, an inner shaft assembly 18, and a nose cone assembly 31 as will be described in more detail below. In some embodiments, the delivery system 10 may not have all of the assemblies disclosed herein. For example, in some embodiments a full mid shaft assembly may not be incorporated into the delivery system 10. In some embodiments, the assemblies may be in a different radial order than is discussed.

Embodiments of the disclosed delivery system 10 may utilize a steerable rail in the rail assembly 20 for steering the distal end of the elongate shaft 12, allowing the implant to be properly located in a patient's body. As discussed in detail below, the steerable rail can be, for example, a rail shaft that extends through the elongate shaft 12 from the handle 14 generally to the distal end of the elongate shaft 12. In some embodiments, the steerable rail has a distal end that ends proximal to the implant retention area 16. A user can manipulate the bending of the distal end of the rail, thereby bending the rail in a particular direction. In preferred embodiments, the rail has more than one bend along its length, thereby providing multiple directions of bending. The rail may deflect the elongate shaft 12 in at least two planes. As the rail is bent, it presses against the other assemblies to bend them as well, and thus the other assemblies of the elongate shaft 12 can be configured to steer along with the rail as a cooperating single unit, thus providing for full steerability of the distal end of the elongate shaft 12.

Once the rail is steered into a particular location in a patient's body, the implant 70 can be advanced along or relative to the rail through the movement of the other sheaths/shafts relative to the rail and released into the body. For example, the rail can be bent into a desired position within the body, such as to direct the implant 70 towards the native mitral valve. The other assemblies (e.g., the outer sheath assembly 22, the mid shaft assembly 21, the inner assembly 18, and the nose cone assembly 31) can passively follow the bends of the rail. Further, the other assemblies (e.g., the outer sheath assembly 22, the mid shaft assembly 21, the inner assembly 18, and the nose cone assembly 31) can be advanced together (e.g., relatively together, sequentially, simultaneously, almost simultaneously, at the same time, closely at the same time) relative to the rail while maintaining the implant 70 in the compressed position without releasing or expanding the implant 70 (e.g., within the implant retention area 16). The other assemblies (e.g., the outer sheath assembly 22, the mid shaft assembly 21, the inner assembly 18, and the nose cone assembly 31) can be advanced distally or proximally together relative to the rail. In some embodiments, only the outer sheath assembly 22, mid shaft assembly 21, and inner assembly 18 are advanced together over the rail. Thus, the nose cone assembly 31 may remain in the same position. The assemblies can be individually, sequentially, or simultaneously, translated relative to the inner assembly 18 in order to release the implant 70 from the implant retention area 16.

FIG. 2C illustrates the sheath assemblies, specifically the outer sheath assembly 22, the mid shaft assembly 21, the inner shaft assembly 18, and the nose cone assembly 31 having translated distally together along the rail assembly 20. In some embodiments, the outer sheath assembly 22, the mid shaft assembly 21, the inner shaft assembly 18, and the nose cone assembly 31 translate together (e.g., relatively together, sequentially with one actuator, simultaneously, almost simultaneously, at the same time, closely at the same time). This distal translation can occur while the implant 70 remains in a compressed configuration within the implant retention area 16.

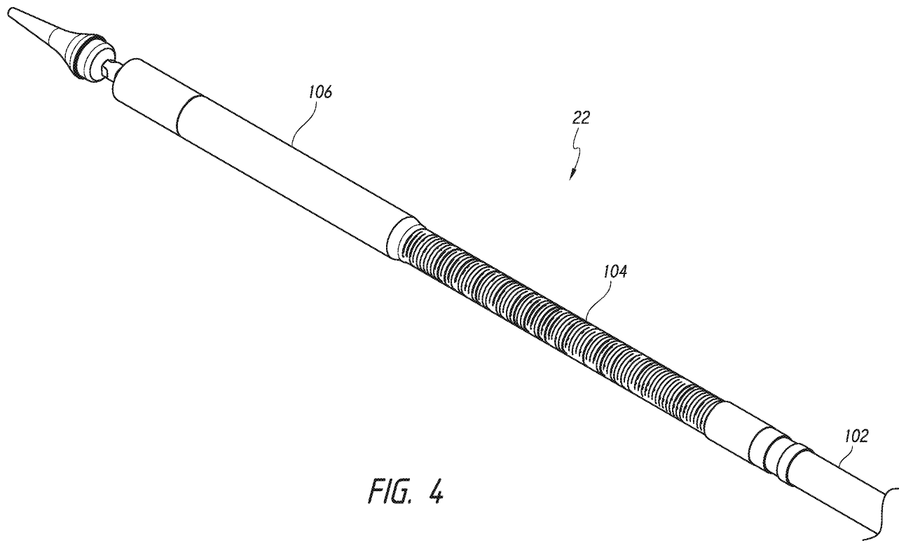

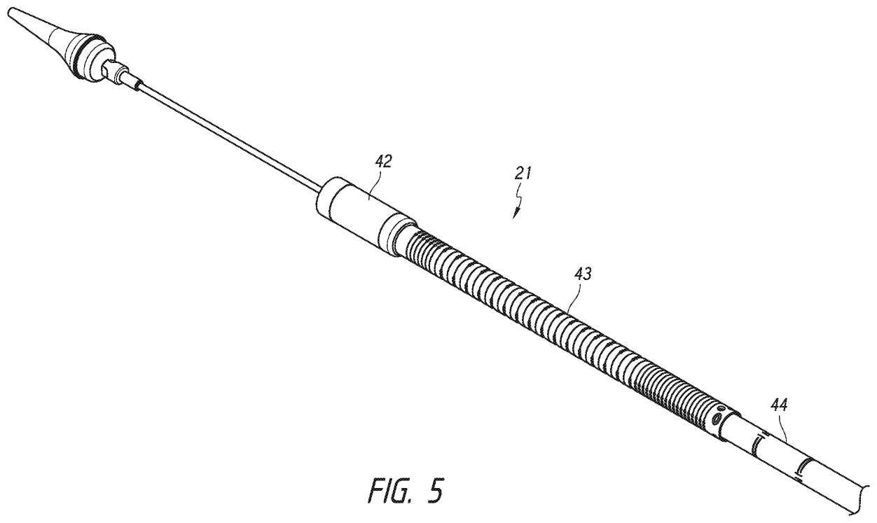

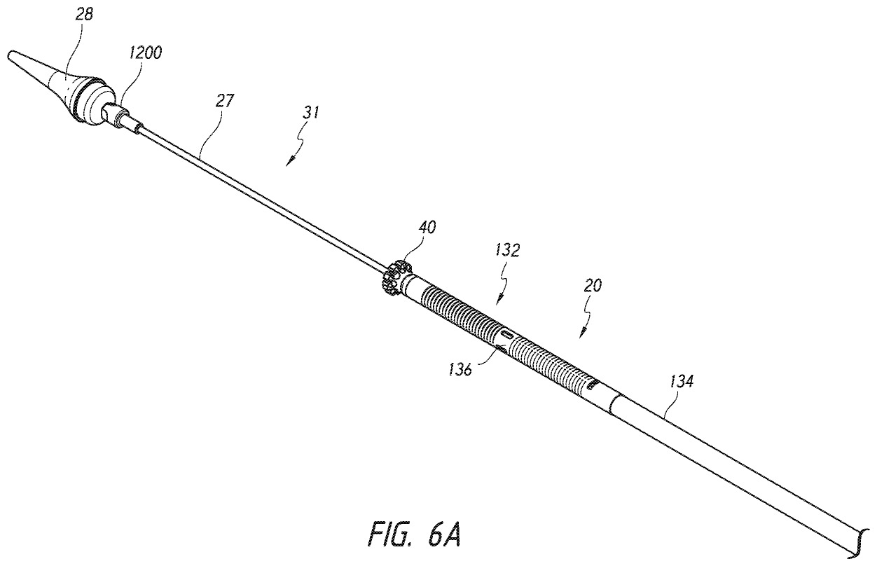

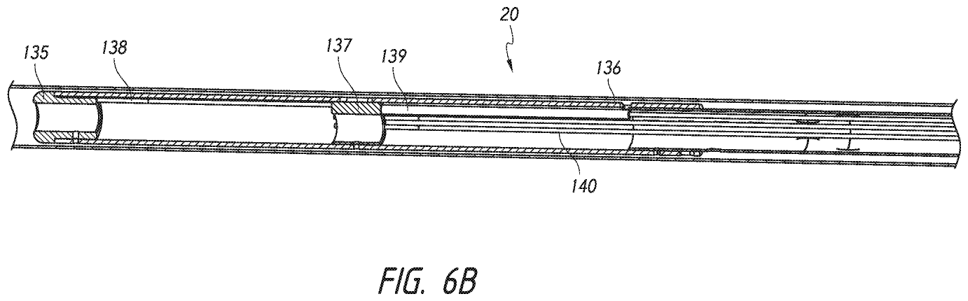

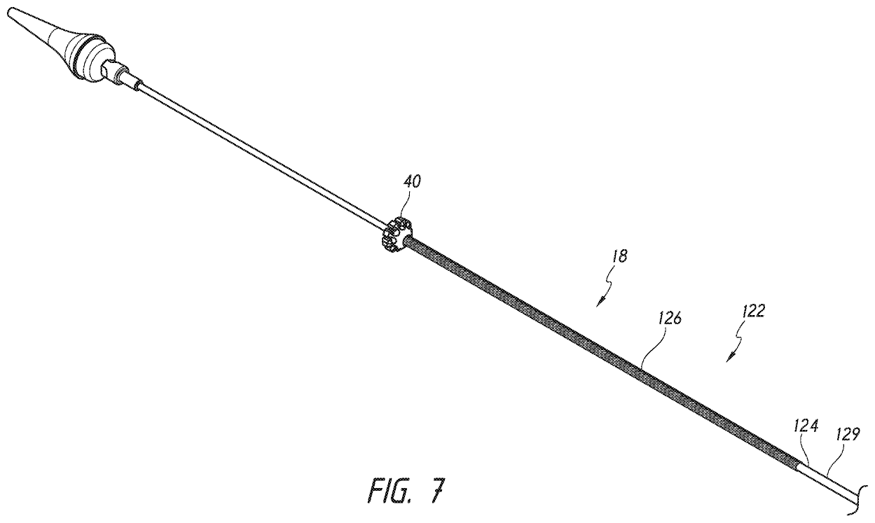

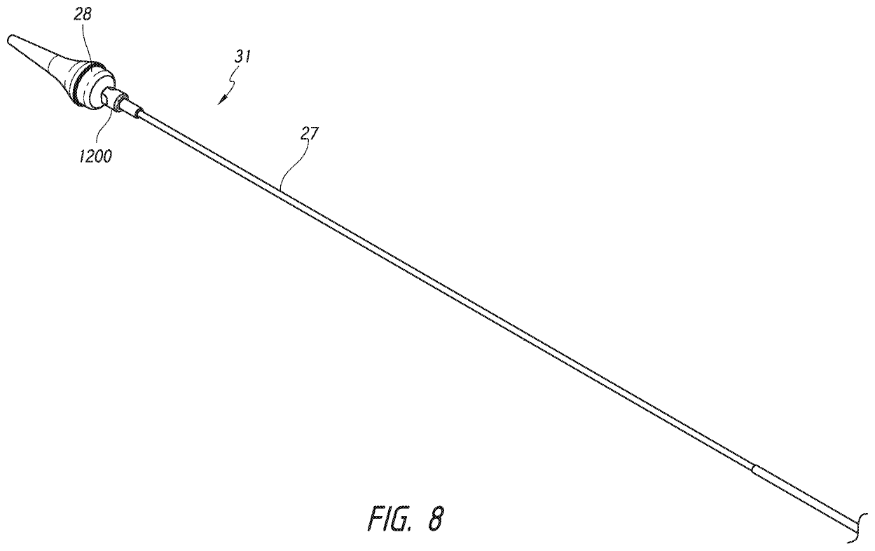

As shown in FIGS. 2A-2C and as further shown in FIG. 4-8, starting with the outermost assembly, the delivery system may include an outer sheath assembly 22 forming a radially outer covering, or sheath, to surround an implant retention area 16 and prevent the implant from radially expanding. Specifically, the outer sheath assembly 22 may prevent radial expansion of the distal end of the implant from radially expanding. Moving radially inward and referring to FIG. 5, the mid shaft assembly 21 may be composed of a mid shaft hypotube 43 with its distal end attached to an outer retention member 42 or outer retention ring for radially retaining a portion of the implant in a compacted configuration, such as a proximal end of the implant 70. The mid shaft assembly 21 may be located within an interior lumen of the outer sheath assembly 22. Moving further inwards, and referring to FIG. 6A, the rail assembly 20 may be configured for steerability, as mentioned above and further described below. The rail assembly 20 may be located within an interior lumen of the mid shaft assembly 21. Moving further inwards and referring to FIG. 7, the inner shaft assembly 18 may be composed of an inner shaft with its distal end attached to inner retention member or inner retention ring 40 (such as a PEEK ring) for axially retaining the implant, for example the proximal end of the implant. The inner shaft assembly 18 may be located within an interior lumen of the rail assembly 20. Further, and referring to FIG. 8, the most radially-inward assembly may be the nose cone assembly 31 which includes the nose cone shaft 27 having its distal end connected to the nose cone 28. The nose cone 28 can have a tapered tip and forms the tip of the elongate shaft 12. The nose cone assembly 31 is preferably located within an interior lumen of the inner shaft assembly 18. The nose cone assembly 31 may include an interior lumen for a guide wire to pass therethrough.

The elongate shaft 12 and its assemblies, more specifically the nose cone assembly 31, inner assembly 18, rail assembly 20, mid shaft assembly 21, and outer sheath assembly 22, can be collectively configured to deliver an implant 70 positioned within the implant retention area 16 (shown in FIG. 2A) to a treatment location. One or more of the assemblies may then be moved to allow the implant 70 to be released at the treatment location. For example, one or more of the assemblies may be movable with respect to one or more of the other assemblies. The implant 70 may be controllably loaded onto the delivery system 10 and then later deployed within the body. Further, the handle 14 can provide steering to the rail assembly 20, providing for bending/flexing/steering of the distal end of the elongate shaft 12.

Referring to FIGS. 2A-2C, the inner retention member 40, the outer retention member 42, and the outer sheath assembly 22 can cooperate to hold the implant 70 in a compacted configuration. In FIG. 2A, the inner retention member 40 is shown engaging struts 72 (marked in FIG. 3A) at the proximal end 301 of the implant 70. For example, slots located between radially extending teeth on the inner retention member 40 can receive and engage the struts 72 which may end in mushroom-shaped tabs 74 on the proximal end of the implant 70 (marked in FIG. 3A). The mid shaft assembly 21 can be positioned over the inner retention member 40 so that the first end 301 of the implant 70 (marked in FIG. 3A) is trapped between the inner retention member 40 and the outer retention member 42, thereby securely attaching it to the delivery system 10 between the mid shaft assembly 21 and the inner retention member 40. The outer sheath assembly 22 can be positioned to cover the second end 303 of the implant 70 (marked in FIG. 3A).

The outer retention member 42 may be attached to a distal end of the mid shaft hypotube 43 which can in turn be attached to a proximal tube 44 at a proximal end (marked in FIG. 5), which in turn can be attached at a proximal end to the handle 14. The outer retention member 42 can provide further stability to the implant 70 when in the compressed position. The outer retention member 42 can be positioned over the inner retention member 40 so that the proximal end of the implant 70 is trapped therebetween, securely attaching it to the delivery system 10. The outer retention member 42 can encircle a portion of the implant 70, in particular the first end 301, thus preventing the implant 70 from expanding. Further, the mid shaft assembly 21 can be translated proximally with respect to the inner assembly 18 into the outer sheath assembly 22, thus exposing a first end 301 of the implant 70 held within the outer retention member 42. In this way the outer retention member 42 can be used to help secure an implant 70 to or release it from the delivery system 10. The outer retention member 42 may have a cylindrical or elongate tubular shape, and may be referred to as an outer retention ring, though the particular shape is not limiting.

The mid shaft hypotube 43 itself (marked in FIG. 5) can be made of, for example, high density polyethylene (HDPE), as well as other appropriate materials as described herein. The mid shaft hypotube 43 can be formed of a longitudinally pre-compressed HDPE tube, which can provide certain benefits. For example, the pre-compressed HDPE tube can apply a force distally onto the outer retention member 42, thus preventing accidental, inadvertent, and/or premature release of the implant 70. Specifically, the distal force by the mid shaft hypotube 43 keeps the distal end of the outer retention member 42 distal to the inner retention member 40, thus preventing the outer retention member 42 from moving proximal to the inner retention member 40 before it is desired by a user to release the implant 70. This can remain true even when the elongate shaft 12 is deflected at a sharp angle.

As shown in FIG. 2A, the distal anchors 80 (marked in FIG. 3A) can be located in a delivered configuration where the distal anchors 80 point generally distally (as illustrated, axially away from the main body of the implant frame and away from the handle of the delivery system). The distal anchors 80 can be restrained in this delivered configuration by the outer sheath assembly 22. Accordingly, when the outer sheath 22 is withdrawn proximally, the distal anchors 80 can flip positions (e.g., bend approximately 180 degrees) to a deployed configuration (e.g., pointing generally proximally). FIG. 2A also shows the proximal anchors 82 extending distally in their delivered configuration within the outer sheath assembly 22. In other embodiments, the distal anchors 80 can be held to point generally proximally in the delivered configuration and compressed against the body of the implant frame.

The delivery system 10 may be provided to users with an implant 70 preinstalled. In other embodiments, the implant 70 can be loaded onto the delivery system 10 shortly before use, such as by a physician or nurse.

FIGS. 4-8 illustrate further views of delivery system 10 with different assemblies translated proximally and described in detail.

Starting with the outermost assembly shown in FIG. 4, the outer sheath assembly 22 can include an outer proximal shaft 102 directly attached to the handle 14 at its proximal end and an outer hypotube 104 attached at its distal end. A capsule 106 can then be attached generally at the distal end of the outer hypotube 104. In some embodiments, the capsule 106 can be 28 French or less in size. These components of the outer sheath assembly 22 can form a lumen for the other subassemblies to pass through.

The outer proximal shaft 102 may be a tube and is preferably formed of a plastic, but could also be a metal hypotube or other material. The outer hypotube 104 can be a metal hypotube which in some embodiments may be cut or have slots, as discussed in detail below. The outer hypotube 104 can be covered or encapsulated with a layer of ePTFE, PTFE, or other polymer/material so that the outer surface of the outer hypotube 104 is generally smooth.

A capsule 106 can be located at a distal end of the outer hypotube 104. The capsule 106 can be a tube formed of a plastic or metal material. In some embodiments, the capsule 106 is formed of ePTFE or PTFE. In some embodiments, this capsule 106 is relatively thick to prevent tearing and to help maintain a self-expanding implant in a compacted configuration. In some embodiments the material of the capsule 106 is the same material as the coating on the outer hypotube 104. As shown, the capsule 106 can have a diameter larger than the outer hypotube 104, though in some embodiments the capsule 106 may have a similar diameter as the hypotube 104. In some embodiments, the capsule 106 may include a larger diameter distal portion and a smaller diameter proximal portion. In some embodiments, there may be a step or a taper between the two portions. The capsule 106 can be configured to retain the implant 70 in the compressed position within the capsule 106. Further construction details of the capsule 106 according to various embodiments are discussed below.

The outer sheath assembly 22 is configured to be individually slidable with respect to the other assemblies. Further, the outer sheath assembly 22 can slide distally and proximally relative to the rail assembly 20 together with the mid shaft assembly 21, inner assembly 18, and nose cone assembly 31.

In embodiments, a hydrophilic layer may be applied to the elongate shaft of the delivery system 10, and particularly to the outer sheath assembly 22. The outer surface of the outer sheath assembly 22 may include a hydrophilic layer that may reduce the friction of the outer sheath assembly 22 as the elongate shaft is passed through the patient's vasculature. The hydrophilic layer may cover the entirety of the outer surface of the outer sheath assembly 22, or may only cover the outer surface of the capsule 106, or may cover other components of the delivery system 10 in embodiments.

In embodiments, the hydrophilic layer may be applied to a surface of the outer sheath assembly 22 comprising expanded polytetrafluoroethylene (ePTFE). The ePTFE surface may form an outer surface of the outer sheath assembly 22 that is then covered by the hydrophilic layer such that the hydrophilic layer then comprises the outer surface of the outer sheath assembly 22. A process may be utilized in which a plasma layer serves as an intermediate layer or tie layer between the ePTFE surface and the hydrophilic layer, to bond the hydrophilic layer to the ePTFE surface. As such, in embodiments, the ePTFE outer surface may be provided, and then the plasma layer may be applied to the ePTFE outer surface. The hydrophilic layer may then be applied to the plasma layer (with the plasma layer serving as an intermediate layer).

In embodiments, other intermediate layers or tie layers may be utilized. For example, in embodiments, chemical etching or methods may be utilized as an intermediate layer or tie layer.

In embodiments, the hydrophilic layer may be comprised of reagents from PhotoLink® reagent families of Photo-Polyvinylpyrrolidone (PV), Photo-Polyacrylamide (PA), and Photo-Crosslinker (PR), and the Kollidon® non-photo polymer povidone. In other embodiments, other formulations for hydrophilic layers may be utilized. In embodiments, a plasma layer may comprise a plasma hydroxyl treatment coating that may be comprised of carbon, hydrogen, and oxygen matter. In other embodiments, other forms of plasma layers may be utilized. The hydrophilic layer and/or intermediate or tie layer disclosed herein may be utilized solely, or may be utilized with any of the other apparatuses, systems, or methods disclosed herein.

Moving radially inwardly, the next assembly is the mid shaft assembly 21. FIG. 5 shows a similar view as FIG. 4, but with the outer sheath assembly 22 removed, thereby exposing the mid shaft assembly 21.

The mid shaft assembly 21 can include a mid shaft hypotube 43 generally attached at its proximal end to a mid shaft proximal tube 44, which in turn can be attached at its proximal end to the handle 14, and an outer retention ring 42 located at the distal end of the mid shaft hypotube 43. Thus, the outer retention ring 42 can be attached generally at the distal end of the mid shaft hypotube 43. These components of the mid shaft assembly 21 can form a lumen for other subassemblies to pass through.

Similar to the other assemblies, the mid shaft hypotube 43 and/or mid shaft proximal tube 44 can comprise a tube, such as a hypodermic tube or hypotube (not shown). The tubes can be made from one of any number of different materials including Nitinol, stainless steel, and medical grade plastics. The tubes can be a single piece tube or multiple pieces connected together. Using a tube made of multiple pieces can allow the tube to provide different characteristics along different sections of the tube, such as rigidity and flexibility. The mid shaft hypotube 43 can be a metal hypotube which in some embodiments may be cut or have slots as discussed in detail below. The mid shaft hypotube 43 can be covered or encapsulated with a layer of ePTFE, PTFE, or other material so that the outer surface of the mid shaft hypotube 43 is generally smooth.

The outer retention ring 42 can be configured as a prosthesis retention mechanism that can be used to engage with the implant 70, as discussed with respect to FIG. 2A. For example, the outer retention ring 42 may be a ring or covering that is configured to radially cover the struts 72 on the implant 70. The outer retention ring 42 can also be considered to be part of the implant retention area 16, and may be at the proximal end of the implant retention area 16. With struts or other parts of an implant 70 engaged with the inner retention member 40, discussed below the outer retention ring 42 can cover both the implant 70 and the inner retention member 40 to secure the implant 70 on the delivery system 10. Thus, the implant 70 can be sandwiched between the inner retention member 40 of the inner shaft assembly 18 and the outer retention ring 42 of the mid shaft assembly 21.

The mid shaft assembly 21 is disposed so as to be individually slidable with respect to the other assemblies. Further, mid shaft assembly 21 can slide distally and proximally relative to the rail assembly 20 together with the outer sheath assembly 22, inner shaft assembly 18, and nose cone assembly 31.

Next, radially inwardly of the mid shaft assembly 21 is the rail assembly 20. FIG. 6A shows approximately the same view as FIG. 5, but with the mid shaft assembly 21 removed, thereby exposing the rail assembly 20. FIG. 6B further shows a cross-section of the rail assembly 20 to view the pull wires. The rail assembly 20 can include a rail shaft 132 (or rail) generally attached at its proximal end to the handle 14. The rail shaft 132 can be made up of a rail proximal shaft 134 directly attached to the handle at a proximal end and a rail hypotube 136 attached to the distal end of the rail proximal shaft 134. The rail hypotube 136 can further include an atraumatic rail tip at its distal end. Further, the distal end of the rail hypotube 136 can abut a proximal end of the inner retention member 40, as shown in FIG. 6. In some embodiments, the distal end of the rail hypotube 136 can be spaced away from the inner retention member 40. These components of the rail shaft assembly 20 can form a lumen for the other subassemblies to pass through.

As shown in FIG. 6B, attached to an inner surface of the rail hypotube 136 are one or more pull wires which can be used apply forces to the rail hypotube 136 and steer the rail assembly 20. The pull wires can extend distally from the knobs in the handle 14, discussed below, to the rail hypotube 136. In some embodiments, pull wires can be attached at different longitudinal locations on the rail hypotube 136, thus providing for multiple bending locations in the rail hypotube 136, allowing for multidimensional steering.

In some embodiments, a distal pull wire 138 can extend to a distal section of the rail hypotube 136 and two proximal pull wires 140 can extend to a proximal section of the rail hypotube 136, however, other numbers of pull wires can be used, and the particular amount of pull wires is not limiting. For example, a two pull wires can extend to a distal location and a single pull wire can extend to a proximal location. In some embodiments, ring-like structures attached inside the rail hypotube 136, known as pull wire connectors, can be used as attachment locations for the pull wires, such as proximal ring 137 and distal ring 135. In some embodiments, the rail assembly 20 can include a distal ring 135 may comprise a distal pull wire connector and the proximal ring 137 may comprise a proximal pull wire connector. In some embodiments, the pull wires can directly connect to an inner surface of the rail hypotube 136.

The distal pull wire 138 can be connected (either on its own or through a connector 135 generally at the distal end of the rail hypotube 136. The proximal pull wires 140 can connect (either on its own or through a connector 137) at a location approximately one quarter, one third, or one half of the length up the rail hypotube 136 from the proximal end. In some embodiments, the distal pull wire 138 can pass through a small diameter pull wire lumen 139 (e.g., tube, hypotube, cylinder) attached on the inside of the rail hypotube 136. This can prevent the wires 138 from pulling on the rail hypotube 136 at a location proximal to the distal connection. Further, the lumen 139 can act as compression coils to strengthen the proximal portion of the rail hypotube 136 and prevent unwanted bending. Thus, in some embodiments the lumen 139 is only located on the proximal half of the rail hypotube 136. In some embodiments, multiple lumens 139, such as spaced longitudinally apart or adjacent, can be used per distal wire 138. In some embodiments, a single lumen 139 is used per distal wire 138. In some embodiments, the lumen 139 can extend into the distal half of the rail hypotube 136. In some embodiments, the lumen 139 is attached on an outer surface of the rail hypotube 136. In some embodiments, the lumen 139 is not used.

For the pair of proximal pull wires 140, the wires can be spaced approximately 180° from one another to allow for steering in both directions. Similarly, if a pair of distal pull wires 138 is used, the wires can be spaced approximately 180° from one another to allow for steering in both directions. In some embodiments, the pair of distal pull wires 138 and the pair of proximal pull wires 140 can be spaced approximately 90° from each other. In some embodiments, the pair of distal pull wires 138 and the pair of proximal pull wires 140 can be spaced approximately 0° from each other. However, other locations for the pull wires can be used as well, and the particular location of the pull wires is not limiting. In some embodiments, the distal pull wire 138 can pass through a lumen 139 attached within the lumen of the rail hypotube 136. This can prevent an axial force on the distal pull wire 138 from creating a bend in a proximal section of the rail hypotube 136.

The rail assembly 20 is disposed so as to be slidable over the inner shaft assembly 18 and the nose cone assembly 31. In some embodiments, the outer sheath assembly 22, the mid shaft assembly 21, the inner shaft assembly 18, and the nose cone assembly 31 can be configured to slide together along or relative to the rail assembly 20, such as proximally and distally with or without any bending of the rail assembly 20. In some embodiments, the outer sheath assembly 22, the mid shaft assembly 21, the inner shaft assembly 18, and the nose cone assembly 31 can be configured to retain the implant 70 in a compressed position when they are simultaneously slid along or relative to the rail assembly 20.

Moving radially inwards, the next assembly is the inner shaft assembly 18. FIG. 7 shows approximately the same view as FIG. 6A, but with the rail assembly 20 removed, thereby exposing the inner shaft assembly 18.

The inner shaft assembly 18 can include an inner shaft 122 generally attached at its proximal end to the handle 14, and an inner retention ring 40 located at the distal end of the inner shaft 122. The inner shaft 122 itself can be made up of an inner proximal shaft 124 directly attached to the handle 14 at a proximal end and a distal section 126 attached to the distal end of the inner proximal shaft 124. Thus, the inner retention ring 40 can be attached generally at the distal end of the distal section 126. These components of the inner shaft assembly 18 can form a lumen for the other subassemblies to pass through.

Similar to the other assemblies, the inner proximal shaft 124 can comprise a tube, such as a hypodermic tube or hypotube (not shown). The tube can be made from one of any number of different materials including Nitinol, cobalt chromium, stainless steel, and medical grade plastics. The tube can be a single piece tube or multiple pieces connected together. A tube comprising multiple pieces can provide different characteristics along different sections of the tube, such as rigidity and flexibility. The distal section 126 can be a metal hypotube which in some embodiments may be cut or have slots as discussed in detail below. The distal section 126 can be covered or encapsulated with a layer of ePTFE, PTFE, or other material so that the outer surface of the distal section 126 is generally smooth.

The inner retention member 40 can be configured as an implant retention mechanism that can be used to engage with the implant 70, as discussed with respect to FIG. 2A. For example, the inner retention member 40 may be a ring and can include a plurality of slots configured to engage with struts 72 on the implant 70. The inner retention member 40 can also be considered to be part of the implant retention area 16, and may be at the proximal end of the implant retention area 16. With struts or other parts of an implant 70 engaged with the inner retention member 40, the outer retention ring 42 can cover both the prosthesis and the inner retention member 40 to secure the prosthesis on the delivery system 10. Thus, the implant 70 can be sandwiched between the inner retention member 40 of the inner shaft assembly 18 and the outer retention ring 42 of the mid shaft assembly 21.

The inner shaft assembly 18 is disposed so as to be individually slidable with respect to the other assemblies. Further, the inner assembly 18 can slide distally and proximally relative to the rail assembly 20 together with the outer sheath assembly 22, mid shaft assembly 21, and nose cone assembly 31.

Moving further inwardly from the inner shaft assembly 18 is the nose cone assembly 31 also seen in FIG. 8. This may be a nose cone shaft 27, and in some embodiments, may have a nose cone 28 on its distal end. The nose cone 28 can be made of polyurethane for atraumatic entry and to minimize injury to venous vasculature. The nose cone 28 can also be radiopaque to provide for visibility under fluoroscopy.

The nose cone shaft 27 may include an interior lumen sized and configured to slidably accommodate a guide wire so that the delivery system 10 can be advanced over the guide wire through the vasculature. However, embodiments of the system 10 discussed herein may not use a guide wire and thus the nose cone shaft 27 can be solid. The nose cone shaft 27 may be connected from the nose cone 28 to the handle, or may be formed of different segments such as the other assemblies. Further, the nose cone shaft 27 can be formed of different materials, such as plastic or metal, similar to those described in detail above.

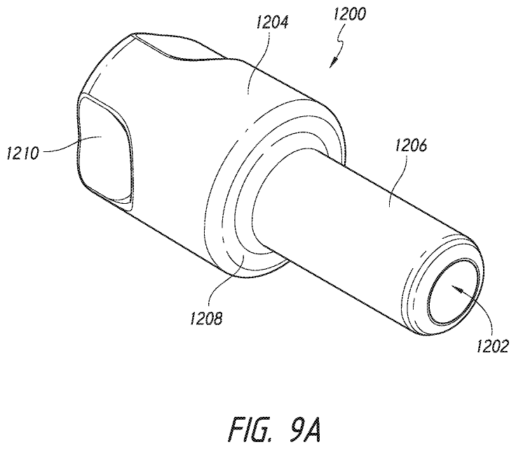

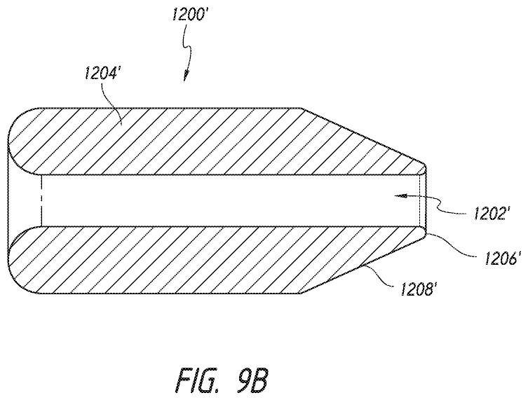

In some embodiments, the nose cone shaft 27 includes a guide wire shield 1200 located on a portion of the nose cone shaft 27. Examples of such a guide wire shield can be found in FIG. 9A-B. In some embodiments, the guide wire shield 1200 can be proximal to the nose cone 28. In some embodiments, the guide wire shield 1200 can be translatable along the nose cone shaft 27. In some embodiments, the guide wire shield 1200 can be locked in place along the nose cone shaft 27. In some embodiments, the guide wire shield 1200 can be at least partially located within the nose cone 28.

Advantageously, the guide wire shield 1200 can allow for smooth tracking of the guide wire with the implant 70 loaded, and can provide a large axial diameter landing zone for a distal end of the implant so that the distal end of the implant 70 may spread out properly and be arranged in a uniform radial arrangement. This uniformity allows for proper expansion. Furthermore, the guide wire shield 1200 can prevent kinking or damaging of the nose cone shaft 27 during compression/crimping of the implant 70, which can exert a large compressive force on the nose cone shaft 27. As the implant 70 can be crimped onto the guide wire shield 1200 instead of directly on the nose cone shaft 27, the guide wire shield 1200 can provide a protective surface.

As shown in FIG. 9A, the guide wire shield 1200 can include a lumen 1202 configured to surround the nose cone shaft 27. The guide wire shield 1200 can include a larger diameter distal end 1204 and a smaller diameter proximal end 1206. In some embodiments, the dimension change between the two ends can be tapered, or can be a step 1208 such as shown in FIG. 9A. The distal end 1204 can include a number of indents 1210 for easier gripping by a user, but may not be included in all embodiments. The proximal end 1206 and the distal end 1204 can both be generally cylindrical, but the particular shape of the guide wire shield 1200 is not limiting.

The distal end of the implant 70 can be crimped so that it is radially in contact with the proximal end 1206 of the guide wire shield 1200. This can allow the implant 70 to be properly spread out around an outer circumference of the proximal end 1206 of the guide wire shield 1200. In some embodiments, the distal end of the implant 70 can longitudinally abut against the proximal end of the distal end 1204 (e.g., at the step 1208), thus providing a longitudinal stop.

FIG. 9B shows an alternate embodiment of a guide wire shield 1200′ having a more tapered configuration. As shown, the proximal end 1206′ of the guide wire shield 1200′ can be a single radially outward taper 1208′ to the distal end 1204′ of the guide wire shield 1200′, which can be generally cylindrical. The guide wire shield 1200′ can also include a lumen 1202′ for receiving the nose cone shaft 27.

The nose cone assembly 31 is disposed so as to be individually slidable with respect to the other assemblies. Further, the nose cone assembly 31 can slide distally and proximally relative to the rail assembly 20 together with the outer sheath assembly 22, mid shaft assembly 21, and inner assembly 18.

In embodiments, the nose cone shaft 27 may be made of a Nitinol material. Such a material may allow for flexibility of the nose cone shaft 27, while allowing the nose cone shaft 27 to be resilient and return back to an unflexed state without kinking or damage to the shaft 27. Further, such a material may be relatively strong, allowing the nose cone shaft 27 to resist forces applied by the delivery system 10 to the shaft 27 during flexing of the delivery system 10 and deployment, or potentially recapture, of an implant from the delivery system 10. In embodiments, the nose cone shaft 27 may comprise a hypotube including cut patterns that may enhance the flexibility of the shaft 27 and reduce the possibility of deformation of the shaft 27.

In embodiments, a nose cone shaft 27 made of a Nitinol material may allow the guide wire shield 1200, 1200′ to be excluded from use. The Nitinol nose cone shaft 27, for example, may resist the force of compression/crimping of the implant 70 upon the shaft 27, and thus the guide wire shield 1200, 1200′ may not be necessary. In embodiments, however, a guide wire shield 1200, 1200′ may yet be utilized with a nose cone shaft 27 made of a Nitinol material. A Nitinol nose cone shaft disclosed herein may be utilized solely, or may be utilized with any of the other apparatuses, systems, or methods disclosed herein.

In some embodiments, one or more spacer sleeves (not shown) can be used between different assemblies of the delivery system 10. For example, a spacer sleeve can be located concentrically between the mid shaft assembly and the rail assembly 20, generally between the mid shaft hypotube 43 and rail hypotube 136. In some embodiments, the spacer sleeve can be generally embedded in the hypotube 43 of the mid shaft assembly 21, such as on an inner surface of the mid shaft assembly 21. In some embodiments, a spacer sleeve can be located concentrically between the rail assembly 20 and the inner assembly 18, generally within the rail hypotube 136. In some embodiments, a spacer sleeve can be used between the outer sheath assembly 22 and the mid shaft assembly 21. In some embodiments, a spacer sleeve can be used between the inner assembly 18 and the nose cone assembly 31. In some embodiments, 4, 3, 2, or 1 of the above-mentioned spacer sleeves can be used. The spacer sleeves can be used in any of the above positions.

The spacer sleeve can be made of a polymer material such as braided Pebax® and can be lined, for example with PTFE, on the inner diameter, though the particular material is not limiting. The spacer sleeve can advantageously reduce friction between the steerable rail assembly 20 and its surrounding assemblies. Thus, the spacer sleeves can act as a buffer between the rail assembly 20 and the inner/nose cone assembly 18/31. Further, the spacer sleeve can take up any gap in radius between the assemblies, preventing compressing or snaking of the assemblies during steering. In some embodiments, the spacer sleeve may include cuts or slots to facilitate bending of the spacer sleeve. In some embodiments, the spacer sleeve may not include any slots, and may be a smooth cylindrical feature.

The spacer sleeve can be mechanically contained by the other lumens and components, and is thus not physically attached to any of the other components, allowing the spacer sleeve to be “floating” in that area. The floating aspect of the spacer sleeve allows it to move where needed during deflection and provide a support and/or lubricious bear surface/surfaces. Accordingly, the floating aspect allows the delivery system 10 to maintain flex forces. However, in some embodiments, the spacer sleeve can be connected to other components.

Each of the outer sheath assembly 22, the mid shaft assembly 21, the inner assembly 18 and the nose cone assembly 31 comprise shafts. Each of the outer sheath assembly 22, the mid shaft assembly 21, and the inner assembly 18 include sheaths having interior lumens. The nose cone assembly 31 comprises a sheath having an interior lumen in an embodiment in which the nose cone assembly 31 includes an interior lumen for a guide wire to extend along.

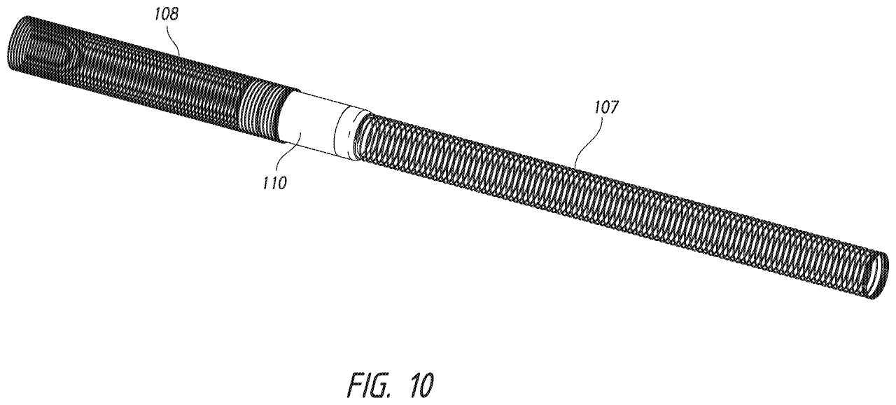

As discussed above, the outer sheath assembly 22, the mid shaft assembly 21, the inner assembly 18, and the rail assembly 20 can contain an outer hypotube 104, a mid shaft hypotube 43, a distal section 126, and a rail hypotube 136, respectively. Each of these hypotubes/sections/shafts can be laser cut to include a number of slots, thereby creating a bending pathway for the delivery system to follow. While different slot assemblies are discussed below, it will be understood that any of the hypotubes can have any of the slot configurations discussed below. FIG. 10-14 show the different hypotubes in isolated format.

The outer hypotube 104, shown in FIG. 10, can be generally formed of a metal coil or a plurality of metal coils. In some embodiments, the outer hypotube 104 can be formed of a proximal metal coil 107 and a distal metal coil 108. The proximal metal coil 107 and the distal metal coil 108 can be longitudinally separated by a tube portion 110, such as shown in FIG. 10. However, in some embodiments the proximal metal coil 107 and the distal metal coil 108 connect. The proximal metal coil 107 and the distal metal coil 108 can be connected to an outer surface of the tube portion 110, for example at the distal end of the proximal metal coil 107 and a proximal end of the distal metal coil 108, in order to form the full outer hypotube 104. In some embodiments, the proximal metal coil 107 and the distal metal coil 108 are generally the same. In some embodiments, the proximal metal coil 107 and the distal metal coil 108 are different, for example in spacing between coils, curvature, diameter, etc. In some embodiments, the distal metal coil 108 has a larger diameter than the proximal metal coil 107, such as when the distal metal coil 108 forms the large diameter of the capsule 106. In some embodiments, they have the same diameter. In some embodiments, one or both of the metal coils 108/107 can form the capsule 106. The coils can be coated by polymer layers, such as described in detail below regarding the capsule construction. The coil construction can allow the outer hypotube 104 to follow the rail in any desired direction.

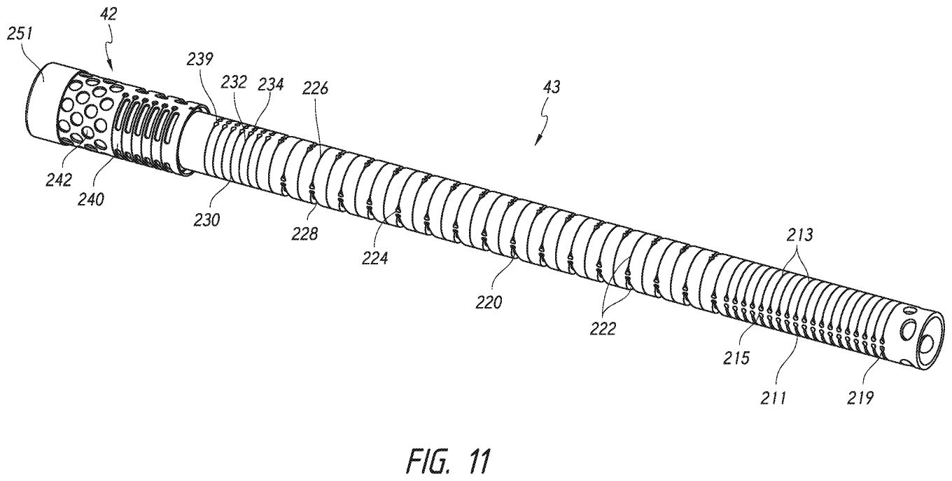

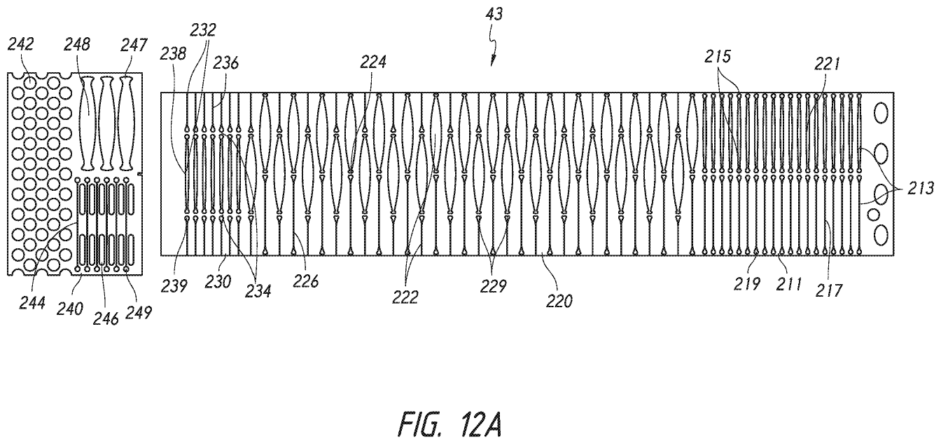

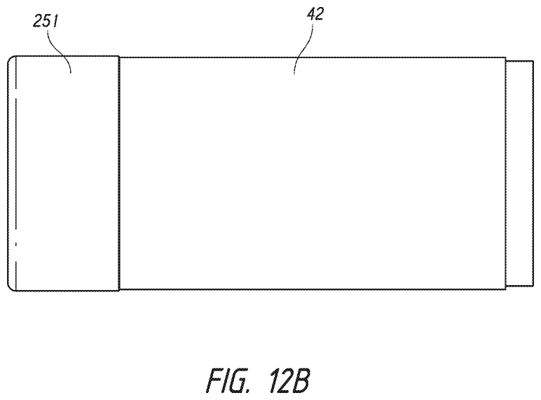

Moving radially inwardly, FIGS. 11-12B shows that the mid shaft hypotube 43 can be a metal laser cut hypotube, such as a lasercut Nitinol hypotube. FIG. 12A illustrates a flat pattern of FIG. 11. As shown in the figures, the hypotube 43 can have a number of cuts forming slots/apertures in the hypotube. In some embodiments, the cut pattern can be the same throughout. In some embodiments, the mid shaft hypotube 43 can have different sections having different cut patterns.

For example, the proximal end of the mid shaft hypotube 43 can be a first section 211 having a plurality of circumferentially extending cut pairs 213 spaced longitudinally along the first section 211. Generally, two slots are cut around each circumferential location forming almost half of the circumference. Accordingly, two backbones or spines 215 are formed between the cuts 213 extending up the length of the first section 211. The cut pairs 213 can be composed of a first thin cut 217. A second cut 221 of each of the cut pairs 213 can be thicker than the first cut 217, such as 1, 2, 3, 4, or 5 times thicker. In some embodiments, the second cut 217 can be generally the same longitudinal thickness throughout the cut. Each of the cuts of the cut pair 213 can end in a teardrop shape 219 in some embodiments to facilitate bending.

Moving distally, the mid shaft hypotube 43 can include a second section 220 having a number of cut pairs 222. Similar to the first section 211, the second section 220 can have a plurality of circumferentially extending cuts spaced longitudinally along the second section 220. Generally, two cuts (e.g., one cut pair 222) are cut around each circumferential location, forming almost half of a circumference. Accordingly, “backbones” 224 can be formed between the cuts extending up the length of the second section 220. Each cut pair 222 can include a first cut 226 that is generally thin and has no particular shape (e.g., it can look the same as the cuts 213 in the first section 211), and a second cut 228 that is significantly longitudinally thicker than the first cut 226. The second cut 228 can be narrower at its ends and longitudinally thicker in its middle portion, thereby forming a curved cut. Moving longitudinally along the second section 220, each cut pair 222 can be offset approximately 45 or 90 degrees as compared to longitudinally adjacent cut pairs 222. In some embodiments, a second cut pair 222 is offset 90 degrees from an adjacent first cut pair 222, and a third cut pair 222 adjacent the second cut pair 222 can have the same configuration of the first cut pair 222. This repeating pattern can extend along a length of the second section 220, thereby providing a particular bending direction induced by the second cut 228 of the cut pairs 222. Accordingly, the “backbone” or spine 224 shifts circumferential position due to the offsetting of adjacent shifting slot pairs 222. Each of the cuts of the cut pair 222 can end in a teardrop shape 229 in some embodiments to facilitate bending.

Moving distally, the mid shaft hypotube 43 can have a third section 230 having a number of cuts. The outer retention ring 240 can be attached to a distal end of the third section 230. The third section 230 can have circumferentially extending cut pairs 232, each cut on the cut pair extending about half way along the circumference to form the two backbones or spines 234. The cut pairs 232 can be composed of a first thin cut 236, similar to the cuts 213 discussed in the first section 211. A second cut 238 of each of the cut pairs 232 can be thicker than the first cut 236, such as 1, 2, 3, 4, or 5 times thicker. In some embodiments, the second cut 238 can be generally the same longitudinal thickness throughout the cut, unlike the second cut 228 of the second section 220. The first cuts 236 and the second cuts 238 can be circumferentially aligned along a length of the third section 230 so that all of the first cuts 236 are in the same circumferential position and all of the second cuts 238 are in the same circumferential position. The second cuts 238 can be aligned with one of the circumferential positions of the second cuts 228 of the second section 220. Each of the cuts of the cut pair 232 can end in a teardrop shape 239 in some embodiments to facilitate bending.

In some embodiments, an outer retention ring strengthener 240 which can partially or fully circumferentially surround the inner retention member 40 can have a number of cuts/slots/holes/apertures as well, such as shown in FIG. 11-12. This can allow it to bend over curves, especially tight curves. In some embodiments, the distal end of the strengthener 240 includes a number of generally circular/elliptical holes 242. This can last for approximately half of the length of the strengthener 240. On the proximal half, one circumferential half of the strengthener 240 can include repeating thin cuts 244 spaced by elongate ovoid holes 246. For example, two circumferentially spaced apart elongate ovoid holes 246 can be between each thin cut 244. Each of the cuts 244 can end in a teardrop shape 249 in some embodiments to facilitate bending. On the other circumferential half of the proximal section, the strengthener 240 can include a number of large cuts 248, for example 1, 2, 3, 4, or 5 large cuts 248 spaced longitudinally apart. The large cuts 248 can be larger in the middle and narrow towards each circumferential end. The large cuts 248 may include ending expansions 247 to facilitate flexibility.

Additionally, the outer retention strengthener 240 can provide strength to lower deployment forces, protect the implant 70 from any metal layers, and can add strength. In some embodiments, the strengthener 240 be a polymer, such as PTFE, though the type of polymer or material is not limiting. In some embodiments, the strengthener 240 can be a metal. In some embodiments, the strengthener 240 can further include an outer polymer layer/jacket, such as a Pebax ® jacket. This prevents the strengthener 240 from catching on the outer sheath assembly 22.

In certain embodiments, the outer retention ring 42 can further include an inner liner for smoothly transitioning over the implant 70. The inner liner can be PTFE or etched PTFE, though the particular material is not limiting and other reduced friction polymers can be used. As shown in FIG. 12B, to prevent delamination during loading of the implant 70, the liner 251 may not be flush at the distal end of the outer retention ring 42. Instead, the liner 251 can be extended and inverted at the distal end in order to cover the distal end of the outer retention ring 42. In some embodiments, the liner 251 can cover an outer surface of the strengthener 240 as well. This can create a seamless rolled reinforced tip of the liner 251. The liner 251 can fully or partially cover an outer surface of the outer retention ring 42, for example ¼, ⅓, ½, ⅔, ¾ (or greater than ¼, ⅓, ½, ¾) , or all of the outer retention ring 42. This solution is advantageous over previously known methods, such as disclosed in U.S. Pat. No. 6,622,367, incorporated by reference in its entirety, as PTFE lined applications do not adhere particularly well to reinforcements or the outer jacket. By inverting the liner 251 and fusing it to the outer retention ring 42 and/or the strengthener 240 and/or an outer polymer jacket on the strengthener 240/outer retention ring 42, this creates a seamless reinforced tip that can mitigate delamination. Delamination is a serious concern because the delaminated liner can tear and embolize during deployment, and the delaminated layer can cause extremely high loading and deployment forces. Delaminated layers can also cause lumen translation problems by locking up shafts thereby adding translational force requirements.

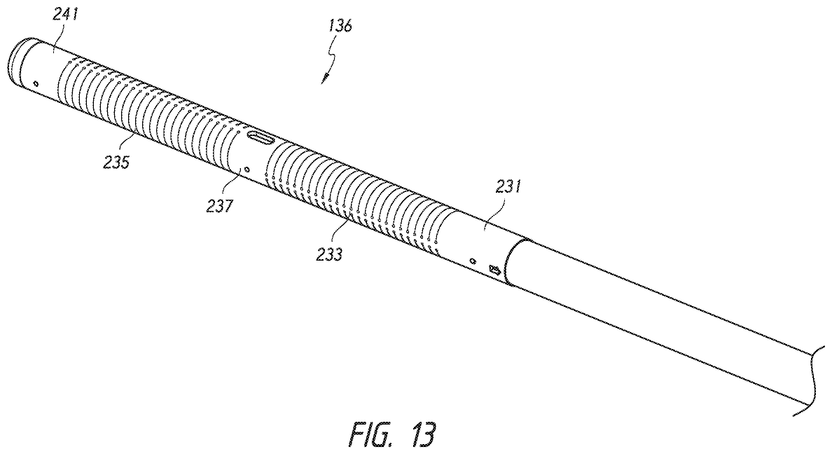

Next, again moving radially inward, FIG. 13 shows an embodiment of the rail hypotube 136 (distal end towards the right). The rail hypotube 136 can also contain a number of circumferential cuts in the form of slots. The rail hypotube 136 can generally be broken into a number of different sections. At the most proximal end is an uncut (or unslotted) hypotube section 231. Moving distally, the next section is the proximal slotted hypotube section 233. This section includes a number of circumferential slots cut into the rail hypotube 136. Generally, two slots are cut around each circumferential location forming almost half of the circumference. Accordingly, two backbones or spines are formed between the cuts extending up the length of the hypotube 136. This is the section that can be guided by the proximal pull wires 140. Moving further distally is the location 237 where the proximal pull wires 140 connect, and thus cuts can be avoided. This section is just distal of the proximally slotted section.

Distally following the proximal pull wire connection area is the distal slotted hypotube section 235. This section is similar to the proximal slotted hypotube section 233, but has significantly more slots cut out in an equivalent length. Thus, the distally slotted hypotube section 235 provides easier bending than the proximally slotted hypotube section 233. In some embodiments, the proximal slotted section 233 can be configured to experience a bend of approximately 90 degrees with a half inch radius whereas the distal slotted section 235 can bend at approximately 180 degrees within a half inch. Further, as shown in FIG. 13, the spines of the distally slotted hypotube section 235 are offset from the spines of the proximally slotted hypotube section 233. Accordingly, the two sections will achieve different bend patterns, allowing for three-dimensional steering of the rail assembly 20. In some embodiments, the spines can be offset 30, 45, or 90 degrees, though the particular offset is not limiting. In some embodiments, the proximally slotted hypotube section 233 can include compression coils. This allows for the proximally slotted hypotube section 233 to retain rigidity for specific bending of the distally slotted hypotube section 235.

At the distalmost end of the distal slotted hypotube section 235 is the distal pull wire connection area 241 which is again a non-slotted section of the rail hypotube 136.

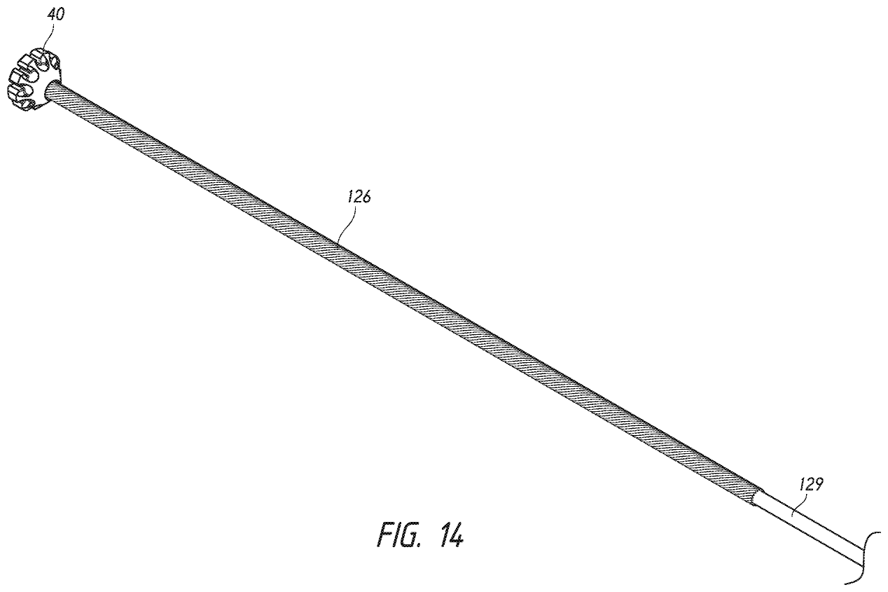

Moving radially inwardly in FIG. 14, the inner assembly 18 is composed generally of two sections. The proximal section is a hypotube 129, either slotted or non-slotted. The distal section 126, which at least partially overlaps an outer surface of the proximal hypotube 129, can be designed to be particularly flexible. For example, the distal section 126 can be more flexible than any of the other shafts discussed herein. In some embodiments, the distal section 126 can be more flexible than any shaft discussed herein other than the nose cone shaft 27. In some embodiments, the distal section 126 can be a flexible tube or hypotube. In some embodiments, the distal section 126 can be a cable, such as a flexible cable. For example, the cable can comprise several strands of wire, such as metal, plastic, polymer, ceramic, etc., wound together to form a rope or cable. Because the cable is so flexible, it can more easily bend with the rail assembly 20. Further, the cable can be smooth, which allows the rail assembly 20 to track over a smooth surface, eliminating the need for any inner liner on the rail assembly 20.

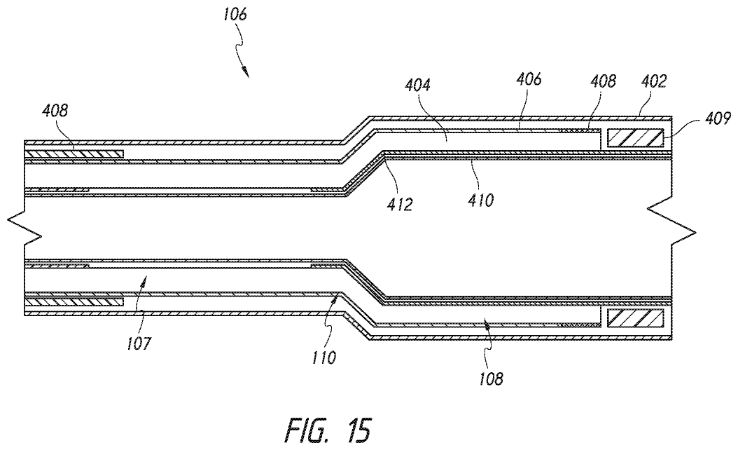

Referring to FIG. 15, the capsule 106 can be formed from one or more materials, such as PTFE, ePTFE, polyether block amide (Pebax®), polyetherimide (Ultem®), PEEK, urethane, Nitinol, stainless steel, and/or any other biocompatible material. The capsule is preferably compliant and flexible while still maintaining a sufficient degree of radial strength to maintain a replacement valve within the capsule 106 without substantial radial deformation, which could increase friction between the capsule 106 and a replacement valve or implant 70 contained therein. The capsule 106 also preferably has sufficient column strength to resist buckling of the capsule, and sufficient tear resistance to reduce or eliminate the possibility of replacement valve tearing and/or damaging the capsule 106. Flexibility of the capsule 106 can be advantageous, particularly for a transseptal approach. For example, while being retracted along a curved member, for example while tracking over a rail assembly as described herein, the capsule 106 can flex to follow the curved member without applying significant forces upon the curved member, which may cause the curved member to decrease in radius. More specifically, the capsule 106 can bend and/or kink as it is being retracted along such a curved member such that the radius of the curved member is substantially unaffected.

FIG. 15 shows embodiments of a capsule 106 that can be used with embodiments of the delivery system 10. The capsule 106 may include any of the materials and properties discussed above. With many implant capsules, compression resistance and flexibility are typically balanced together, as improved flexibility can lead to worse compression resistance. Thus, there tends to be a choice made between compression resistance and flexibility. However, disclosed are embodiments of a capsule 106 that can achieve both high compression resistance as well as high flexibility. Specifically, the capsule 106 can bend in multiple directions.

In particular, a metal hypotube can provide radial strength and compression resistance, while specific cuts such as slots in the hypotube can enable the flexibility of the capsule 106. In some embodiments, a thin liner and a jacket can surround the capsule 106, such as a polymer layer, to prevent any negative interactions between the implant 70 and the capsule 106.

In some embodiments, the capsule 106 can have a particular construction to allow for it to achieve advantageous properties, as shown in FIG. 15. The capsule 106 can be made of several different layers to provide such properties.

In some embodiments, the capsule 106 can be formed of a metal layer 404, which gives the capsule 106 its structure. This metal layer can include the coils discussed with respect to FIG. 10, or could be one or more hypotubes. The capsule 106 is then covered on an outer surface by a polymer layer and on an inner surface by a liner. All of these features are discussed in detail below.

As mentioned, the metal layer 404 can be, for example, a metal hypotube or laser cut hypotube. In some embodiments, the metal layer 404 can be a metal coil or helix, as discussed in detail above with respect to FIG. 10. Though not limiting, the metal layer 404 can have a thickness of 0.007 inches (or about 0.007 inches).

If a metal coil, such as shown in FIG. 10, is used, the coil dimensions can stay the same throughout a length of the metal layer 404. However, in some embodiments the coil dimensions can vary along a length of the metal layer 404. For example, the coils can vary between coils having a 0.014-inch gap with a 0.021-inch pitch (e.g., small coils), coils having a 0.020 inch-gap with a 0.02-inch pitch (e.g., large coils), and coils having a 0.020-inch gap with a 0.027-inch pitch (e.g., spaced large coils). However, these particular dimensions are merely examples, and other designs can be used as well.

The distalmost end of the metal layer 404 can be formed out of the small coils. Moving proximally, the metal layer 404 may then transition to a section of large coils, followed again by a section of small coils, and then finally the proximalmost section can be the spaced large coils. As an example set of lengths, though not limiting, the distalmost small coil section may have a length of 10 mm (or about 10 mm). Moving proximally, the adjacent large coil section may extend 40 mm (or about 40 mm) to 60 mm (or about 60 mm) in length. These two sections would be found in the distal metal coil 108 shown in FIG. 10. Moving to the proximal metal coil 107 shown in FIG. 10, the small coil section can have a length of 10 mm (or about 10 mm). The remaining portion of the proximal metal coil 107 can be the spaced large coil section. The spaced large coil section can have a length of 40 mm (or about 40 mm) to 60 mm (or about 60 mm) or greater.

As mentioned, the metal layer 404 (either coil or hypotube) can be covered by an outer polymer layer or jacket 402. In some embodiments, the outer polymer 402 layer is an elastomer, though the particular material is not limiting. In some embodiments, the outer polymer layer 402 can comprise polytetrafluoroethylene (PTFE) or expanded polytetrafluoroethylene (ePTFE). The ePTFE can have very different mechanical properties that PTFE. For example, ePTFE can be much more flexible while still maintaining good tensile/elongation properties. In some embodiments, the outer polymer layer 402 can comprise a thermoplastic elastomer, such as PEBAX®. In some embodiments, the outer polymer layer 402 can be pre-axially stressed before applying to the capsule. The outer polymer layer 402 can be approximately 0.006 to 0.008 inches in thickness, but the particular thickness is not limiting.

The outer polymer layer 402 can be applied to the metal layer 404 to form an outer jacket, such as by reflowing the polymer. In some embodiments, the outer polymer layer 402 can be directly applied to the metal layer 404. In some embodiments, an adhesive layer 406 can be disposed between the metal layer 404 and the outer polymer layer 402 to promote attachment of the outer polymer layer to the metal layer. For example, a fluoropolymer, or other soft durometer fluoroelastomer, can be applied between the metal layer 404 and the outer layer 402 in order to attach the two layers together and prevent delamination. In some embodiments, the adhesive layer 406 is not used.

In some implementations, other materials can be included between the metal layer 404 and the outer polymer layer 402 in order to improve properties. For example, fluorinated ethylene propylene (FEP) sections 408 can improve radial strength, in particular when the implant is under compression. While an FEP layer 408 is discussed as a particular material, other high strength polymers, metals, or ceramics can be used as well, and the particular material is not limiting. The FEP layer 408 can also act as an adhesive in some instances.

FEP sections 408 can be included at the distal and proximal ends of the capsule 106. The FEP sections 408 can either overlap the adhesive layer 406. Thus, FEP sections 408 can be located between the adhesive layer 406 and the metal layer 404 or between the adhesive layer 406 and the outer polymer layer 402. In some embodiments, the FEP sections 408 may be located in sections of the capsule 106 that do not include an adhesive layer 406.

The FEP section 408 located at the distal end of the capsule 106 can have a length of 10 mm (or about 10 mm), though the particular length is not limiting. In some embodiments, the FEP section 408 is approximately 0.003 inches in thickness, but the thickness may vary and is not limited by this disclosure. In some embodiments, different FEP sections 408 (e.g., a proximal section and a distal section) can have different thicknesses. In some embodiments, all FEP 408 layers have the same thickness. Example thicknesses can be 0.006 inches or 0.003 inches.

Moving to the inside of the metal layer 404, a liner 410 can be included on its radially inner surface. The liner 410 can be formed of a low friction and/or high lubricity material that allows for the capsule 106 to be translated over the implant 70 without catching or damaging portions of the implant 70. In some embodiments, the liner 410 can be PTFE, which can resist radial expansion and decrease friction with the implant 70.

In some embodiments, the liner 410 is made from ePTFE. However, it can be difficult to reflow a standard ePTFE liner 410 on the inner layer of the capsule 106. Accordingly, the ePTFE liner layer 410 can be pre-compressed before applying onto the inner layer of the capsule 106. In some embodiments, portions of the outer polymer layer 402 and the liner 410 can be in contact with one another. Thus, prior to bonding the two layers together, the ePTFE liner 410 and/or outer polymer layer 402 can be axially compressed. Then, the layers can be bonded together with reflow techniques during manufacturing. For example, the ePTFE liner 410 can be axially compressed, such as over a mandrel, while the outer polymer layer 402 can be placed over it. These two layers can then be reflowed (e.g., melting under pressure) to connect. The combined layers can be slid into and/or around the metal layer 404 discussed herein, and can be melted under pressure again to form the final capsule 106. This technique can allow for the capsule 106 to maintain flexibility and prevent breakage/tearing.

As mentioned above, the inner liner 410 can be ePTFE in some embodiments. The surface friction of ePTFE can be about 15% less than standard PTFE, and can be about 40% less than standard extruded thermoplastics that are used in the art.

In certain embodiments, the liner layer 410 can extend only along an inner surface of the capsule 106 and terminate at a distal end. However, to prevent delamination during loading of the implant 70, the liner 410 may not be flush at the distal end of the capsule 106. Instead, the liner 410 can be extended and inverted at the distal end in order to cover the distal end of the capsule 106 as well as an outer diameter of a portion of the outer polymer layer 402. This can create a seamless rolled reinforced tip of the liner 410. This solution is advantageous over previously known methods, such as disclosed in U.S. Pat. No. 6,622,367, incorporated by reference in its entirety, as PTFE lined applications do not adhere particularly well to reinforcements or the outer jacket. By inverting the liner 410 and fusing it with the outer polymer layer 402, this creates a seamless reinforced capsule tip that can mitigate delamination. Delamination is a serious concern because the delaminated liner can tear and embolize during deployment, and the delaminated layer can cause extremely high loading and deployment forces. Delaminated layers can also cause lumen translation problems by locking up shafts thereby adding translational force requirements.

In some embodiments, another FEP section 412 can be included between the liner 410 and the metal layer 404. The FEP section 412 can be located on distal metal coil 108, as well as the tube 110 transitioning between the distal metal coil 108 and the proximal metal coil 107. In some embodiments, the FEP section 412 may continue partially or fully into the proximal metal coil 107.

In some embodiments, an FEP section 412 can be included in the proximalmost portion of the proximal metal coil 107. This FEP section 412 be approximately 0.5 inches in length. In some embodiments, there is a longitudinal gap between the proximalmost FEP section 412 and the FEP section 412 that extends over the distal metal coil 108. In some embodiments, the previously mentioned FEP sections 412 are continuous.

As shown in FIG. 15, the metal layer 404 may stop proximal to the edges of the outer polymer layer 402, liner 410, and FEP section 412. If so, a thicker portion of an adhesive layer 409 can be applied at the distal end of the metal layer 404 to match the distal end of the other layers. However, this section can be removed during manufacture, so the distal end of the metal layer 404 is the distal end of the capsule 106, which can then be covered by the liner 410. In some embodiments, the extended sections distal to the metal layer 404 are not used.

In embodiments, a covering layer may be applied to the capsule 106 that may include reinforcing fibers or beads. A delivery system accordingly may include an elongate shaft, having a proximal end and a distal end, and including an implant retention area configured to retain the implant. A covering layer may be on the elongate shaft and may include reinforcing fibers or beads. The covering layer may form an interior liner of the elongate shaft. The covering layer may comprise the interior liner 410 of the capsule 106, or a liner of another capsule as disclosed herein, or may be a liner of another portion of the delivery system 10. For example, the covering layer may comprise a liner extending along the interior of the outer sheath assembly 22 from the capsule 106 back to the handle of the delivery system 10, and thus may form an interior liner of the outer sheath. In embodiments, the covering layer may be applied to other components of the delivery system 10, including the mid shaft assembly, or the interior shaft assembly, among other components. In embodiments, the covering layer may be applied to as an outer layer of one or more of the components.

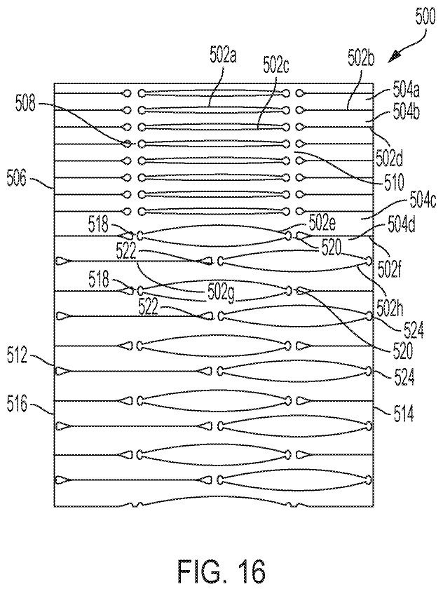

The covering layer may include the reinforcing fibers or beads to provide strength for the covering layer. For example, the covering layer may include a material such as polytetrafluoroethylene (PTFE) that is mixed with the reinforcing fibers or beads. The PTFE may provide a lubricious surface that is then reinforced with the reinforcing fibers or beads for strength. The tensile strength of the covering layer, for example, may be improved with the use of the reinforcing fibers or beads. Further, in embodiments as shown in FIG. 16-21, in which a hypotube including cut patterns is utilized in a capsule, the reinforcing fibers or beads may serve to protect an implant from the cut patterns of the hypotube. The reinforcing fibers or beads may comprise glass, and in embodiments may comprise silicate fibers or beads or carbon (e.g., graphite) fibers or beads.

The covering layer may further provide toughness and durability to protect any of the hypotube cut patterns (e.g., a laser cut member) from binding during relative translation. For example, as a cut hypotube of the capsule is retracted or advanced relative to a cut hypotube of the mid-assembly, then there may be a reduced possibility of such hypotubes binding during relative translation.

The covering layer may be formed by a mixture of the base material (e.g., PTFE) with the reinforcing fibers or beads. For example, PTFE pellets may be mixed with the reinforcing fibers or beads in a desired proportion. The mixture may be heated and then extruded to form the covering layer. The remainder of the delivery system 10, for example, the capsule 106 and other portion of the outer sheath assembly 22 may be formed with the covering layer. For example, the covering layer may be applied as an interior liner for the capsule 106.

The proportion of the base material (e.g., PTFE) to the reinforcing fibers or beads may be set as desired. In embodiments, the proportion may be a mixture of the base material (e.g., PTFE) and the reinforcing fibers and beads in which between 1% to 10%, inclusive, comprises the reinforcing fibers or beads. In embodiments, the proportion may be a mixture of the base material (e.g., PTFE) and the reinforcing fibers and beads in which 0.5% to 25%, inclusive, comprises the reinforcing fibers or beads. In embodiments, the proportion may be a mixture of the base material (e.g., PTFE) and the reinforcing fibers and beads in which 0.1% to 30%, inclusive, comprises the reinforcing fibers or beads. In embodiments, the proportion may be a mixture of the base material (e.g., PTFE) and the reinforcing fibers and beads in which up to 25% or 30%, inclusive comprises the reinforcing fibers or beads.

A method may include deploying the elongate shaft to a location within a patient's body, the elongate shaft including an implant retention area retaining an implant for implantation within the patient's body and a covering layer including reinforcing fibers or beads. The method may include retracting a capsule surrounding the implant retention area, with the covering layer forming an interior liner of the capsule. The method may include sliding the interior liner along the implant, which may reduce friction with the implant and provide a tough and durable interior liner.

A method may include providing the elongate shaft and preparing the covering layer including reinforcing fibers or beads. The method may include preparing a mixture of PTFE with the reinforcing fibers or beads, or another base material. The mixture may be extruded with the PTFE including the reinforcing fibers or beads. The extrusion may be provided with the remainder of the elongate shaft, and may comprise a liner of the elongate shaft such as an interior liner of a capsule or another portion of the outer sheath. The covering layer disclosed herein may be utilized solely, or may be utilized with any of the other apparatuses, systems, or methods disclosed herein.

FIG. 16 illustrates an embodiment of a hypotube 500 shown in a flattened configuration that may be utilized with assemblies of the present disclosure. The hypotube 500 may be utilized as a metal layer, or metal portion of one or more of the assemblies disclosed herein, and particularly may comprise a portion of a capsule 106 as shown in FIG. 4. The hypotube 500 may be utilized with a capsule 106 that is configured to surround an implant retention area 16 of the elongate shaft 12. The hypotube 500 may be utilized as a metal layer 404 for a capsule as shown in FIG. 15, and may be utilized in other portions of the assemblies that utilize hypotubes, or other portions of the assemblies. The hypotube 500 may be positioned between an outer jacket and a liner layer disclosed herein, among other components disclosed in embodiments herein. Although shown in a flattened configuration in FIG. 16, the hypotube 500 has outer sides 512, 514 that when connected form a cylindrical shape for the hypotube 500 in a similar manner as the hypotube shown in FIG. 11.

The hypotube 500 may have one or more cuts 502a-h that form a plurality of rings 504a-d of the hypotube 500. The cuts 502a-h may be in shape of slots as shown in FIG. 16 or may have other shapes as desired. The plurality of rings 504a-d may be spaced longitudinally from each other and extend circumferentially around the hypotube 500.

The hypotube 500 may include a first section 506 that may be a distal portion of the hypotube 500. The distal portion of the hypotube 500 may form a distal portion of the capsule 106, or a distal portion of the assembly that the hypotube 500 is utilized with. The first section 506 may include cut pairs (502a and 502b form a pair, with 502c and 502d forming another pair). The cuts 502a and 502c may be aligned longitudinally, at the same circumferential position, and the cuts 502b and 502d may be aligned longitudinally, at the same circumferential position, such that spines 508, 510 may be positioned between circumferentially spaced cuts 502a, b. The spines 508, 510 may extend longitudinally along the hypotube 500.

The hypotube 500 is shown in FIG. 16 in a flattened configuration, and as such, when the outer sides 512, 514 of the hypotube 500 are connected such that the hypotube 500 forms a cylinder, the first section 506 will include a set of circumferentially extending cuts 502a, 502c on one side of the hypotube 500 and a set of circumferentially extending cuts 502b, 502d on the opposing side of the hypotube 500. Each of the cuts disclosed in this application may end in a teardrop shape to facilitate bending.

Each cut in the pair 502a, b may extend circumferentially around almost half of the circumference of the hypotube 500, and similarly each cut in the pair 502c, d may extend circumferentially around almost half of the circumference of the hypotube 500. One cut 502a, c in a pair may be circumferentially spaced from the other cut and have a greater longitudinal size or thickness than the other cut 502b, d in the pair. The thickness of the cut 502a, c may be 1, 2, 3, 4, or 5 times thicker than the thickness of the other cut in the pair 502b, d, among other thicknesses. The cuts 502a, 502c having a greater longitudinal size or width than the other cuts 502b, 502d will cause the section 506 to have a bias to flex in a direction towards the cuts 502a, 502c and away from the cuts 502b, 502d. The spines 508, 510 accordingly may extend along the neutral axis of bend when the first section 506 is bent.

The first section 506 accordingly may comprise a section of the hypotube 500 biased to flex in a single direction.

The hypotube 500 may include a second section 516. The second section 516 may be positioned proximal of the first section 506 and may comprise a proximal portion of the capsule 106 or other proximal portion of an assembly that the hypotube 500 is utilized with.

The second section 516 may include longitudinally spaced rows of cuts 502e-h that form rings 504c-d of the hypotube 500 that are spaced longitudinally from each other and extend circumferentially around the hypotube 500.

The cuts 502e-h may comprise pairs of cuts 502e, f and 502g, h. Each cut in the pair 502e, f may extend circumferentially around almost half of the circumference of the hypotube 500, and similarly each cut in the pair 502g, h may extend circumferentially around almost half of the circumference of the hypotube 500.

One cut 502e, h in the pair may have a greater longitudinal size or thickness than the other cut 502f, g in the pair. The thickness of the cut 502e, h may be 1, 2, 3, 4, or 5 times longitudinally thicker than the thickness of the other circumferentially spaced cut 502f, g in the pair. The pairs of cuts 502e, f and 502g, h may be offset from each other circumferentially, and may be offset from each other by about 90 degrees as shown in FIG. 16, or by a different offset as desired. As such, the pairs of cuts 502e, f and 502g, h may form two pairs of spines 518, 520 and 522, 524, with the spines 518, 520 longitudinally aligned with spines 508, 510 of the first section 506. The pair of spines 518, 520 may be offset from the pair of spines 522, 524 by about 90 degrees as shown in FIG. 16, or by a different offset as desired. The spines within each pair may be positioned 180 degrees from each other. Such a configuration may be formed by a repeating pattern of staggered cuts comprising the pairs of cuts 502e, f and 502g, h being offset from each other along the length of the hypotube 500. The position of the spines 518, 520 and 522, 524, and the increased longitudinal size or width of the cuts 502e, h will cause the section 516 to have a bias to flex in two directions (a first direction and a second direction), one towards the cuts 502e and one towards the cuts 502h. The spines 518, 520 and 522, 524 may each accordingly extend along the neutral axis of bend when the second section 516 is bent. A greater number of directions of bend may be provided if desired (e.g., at least two directions of bend).

The hypotube 500 may provide support for an assembly of the delivery system 10 and may provide support for the capsule 106. The plurality of cuts shown in FIG. 16 may allow for flexibility of the capsule 106, particularly in the directions of bias formed by the cut patterns.

The plurality of rings 504a-d of the hypotube 500 may provide for support of the capsule 106. In addition, the plurality of rings 504a-d may allow for a force to be applied by the capsule 106 in a distal direction. Such a force may be applied in an embodiment in which the capsule 106 is utilized for recapture of an implant that may have been partially deployed from the implant retention area 16 (as shown in FIG. 2C). For example, if the capsule 106 has been retracted to deploy the implant from the implant retention area 16, it may be desired to retrieve the partially deployed implant. The capsule 106 may then be forced distally to recapture the partially deployed implant and possibly return the implant to the implant retention area 16. The structural strength provided by the hypotube 500 may allow this distal force to be applied to the partially deployed implant for recapture. The plurality of rings 504a-d of the hypotube 500 may compress against each other to allow the distal force to be applied.

Further, the biased flexibility provided by the plurality of cuts may allow the hypotube 500, and accordingly the capsule 106, to flex to accommodate bend of the capsule 106 which may include bending caused by translation along a bent rail assembly. The first section 506 may be biased to bend in a single direction to accommodate that direction of bend of the rail assembly. The second section 516 may be biased to bend in two directions (or at least two directions) to accommodate two directions of bend (or at least two directions of bend) of the rail assembly.

The configuration of the hypotube 500, including use of the plurality of rings 504a-d , may allow for an improved distal force to be applied by the capsule 106 or another portion of the assembly utilizing the hypotube 500. The plurality of rings 504a-d may contact and press against each other to allow the distal force to be applied. Notably, however, the directions of bend of the first section 506 and the second section 516 should be aligned with the directions of bend of the rail assembly or other bend of the elongate shaft 12 to allow for desired bend of the hypotube 500. As such, it may be beneficial to produce a hypotube configuration that lacks a flex bias in a direction.

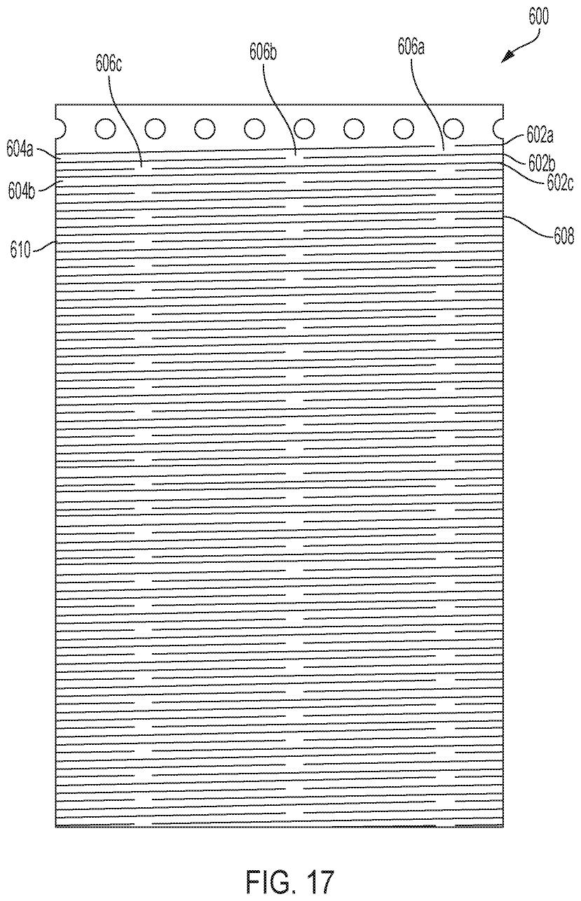

FIG. 17 illustrates an embodiment of a hypotube 600 shown in a flattened configuration that may be utilized with assemblies of the present disclosure. The hypotube 600 may be utilized as a metal layer, or metal portion of one or more of the assemblies disclosed herein, and particularly may comprise a portion of a capsule 106 as shown in FIG. 4. The hypotube 600 may be utilized with a capsule 106 that is configured to surround an implant retention area 16 of the elongate shaft 12. The hypotube 600 may be utilized as a metal layer 404 for a capsule as shown in FIG. 15, and may be utilized in other portions of the assemblies that utilize hypotubes, or other portions of the assemblies. The hypotube 600 may be positioned between an outer jacket and a liner layer disclosed herein, among other components disclosed in embodiments herein. Although shown in a flattened configuration in FIG. 17, the hypotube 600 has outer sides 608, 610 that when connected form a cylindrical shape for the hypotube 600 in a similar manner as the hypotube shown in FIG. 11.

The hypotube 600 has one or more cuts 602a-c that form a plurality of rings 604a-b of the hypotube 600. The plurality of rings 604a-b may be longitudinally spaced from each other and form a pattern of rings extending circumferentially for the length of the hypotube 600. The one or more cuts represented as 602a-c shown in FIG. 17 are cut into a spiral that extends circumferentially around the hypotube 600 for the length of the hypotube 600. The cuts may be separated by spines 606a, 606b, 606c that extend longitudinally along the hypotube 600 and connect the plurality of rings 604a-b. The spines may be offset circumferentially from each other. The spines 606a, 606b, 606c may be positioned such that a single cut of the cut pattern extends more than 360 degrees around the hypotube 600.

The spiral configuration of the hypotube 600 may allow the hypotube 600 to lack a bias in a direction of bend. The hypotube 600 accordingly may be configured to bend in multiple directions without a particular bias towards a direction and may have equal flexibility in all radial directions. Such a feature may allow the hypotube 600 to have a greater variety of orientations about the directions of bend of the rail assembly, as the hypotube 600 lacks a particular bias towards one direction of bend of the rail assembly. The spiral configuration may allow the hypotube 600 to remain flexible, to flex in various directions of bend as desired.

Further, the spiral configuration of the hypotube 600 may allow the plurality of rings 604a-b to more strongly contact and compress against each other to transmit a distal force for implant recapture or the like. The gaps formed by the cuts 602a-c may be reduced in size when the hypotube 600 is distally compressed, such that a rigid rod structure is formed for recapture of the implant. The lack of bias of the hypotube 600 may also reduce the possibility of the hypotube 600 deflecting in a direction due to a flex bias of the hypotube 600.

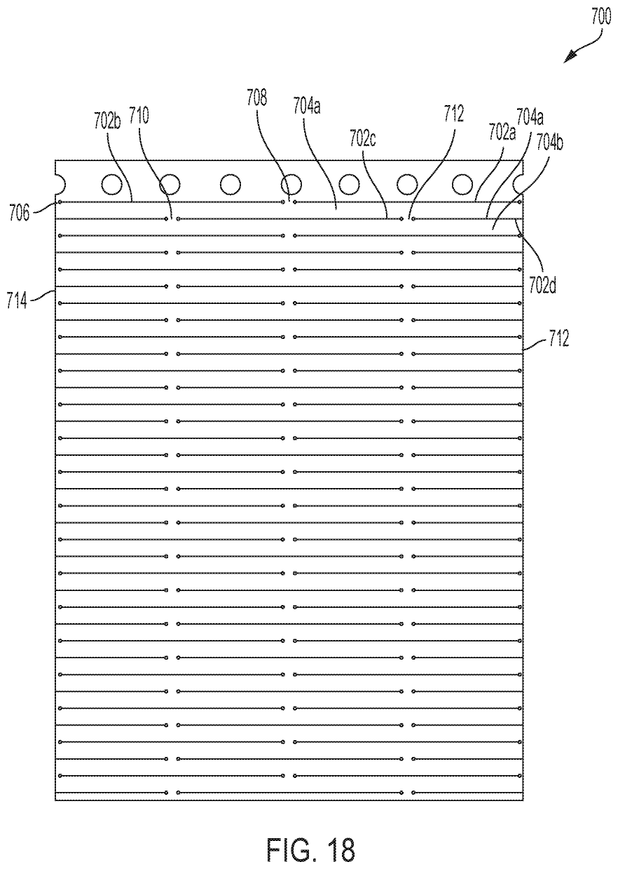

FIG. 18 illustrates an embodiment of a hypotube 700 shown in a flattened configuration that may be utilized with assemblies of the present disclosure. The hypotube 700 may be utilized as a metal layer, or metal portion of one or more of the assemblies disclosed herein, and particularly may comprise a portion of a capsule 106 as shown in FIG. 4. The hypotube 700 may be utilized with a capsule 106 that is configured to surround an implant retention area 16 of the elongate shaft 12. The hypotube 700 may be utilized as a metal layer 404 for a capsule as shown in FIG. 15, and may be utilized in other portions of the assemblies that utilize hypotubes or other portions of the assemblies. The hypotube 700 may be positioned between an outer jacket and a liner layer disclosed herein, among other components disclosed in embodiments herein. Although shown in a flattened configuration in FIG. 18, the hypotube 700 has outer sides 712, 714 that when connected form a cylindrical shape for the hypotube 700 in a similar manner as the hypotube shown in FIG. 11.

The hypotube 700 has one or more cuts 702a-d that form a plurality of rings 704a-b of the hypotube 700. The plurality of rings 704a-b may be longitudinally spaced from each other and form a pattern of rings extending circumferentially for the length of the hypotube 700.

The one or more cuts 702a-d shown in FIG. 18 are cut into pairs (cuts 702 a and 702 b form a pair, and cuts 702 c and 702 d form a pair) that are offset from each other. The cuts 702a-d may have equal longitudinal widths, and may have equal circumferential lengths. The one or more cuts 702a-d may form a repeating pattern of staggered cuts that have an equal size. The cuts may be separated by pairs of spines (706 and 708 form a pair, and 710 and 712 form a pair) that are offset from each other and each extend longitudinally along the hypotube 700. Each spine in the pair may be offset 180 degrees from the other spine in the pair. The amount of offset of the pairs of spines may be 90 degrees as shown in FIG. 18, or another amount as desired.

The offset configuration of the cuts 702a-d of the hypotube 700 may allow the hypotube 700 to lack a bias in a direction of bend. The hypotube 700 accordingly may be configured to bend in a multitude of directions without a particular bias towards a direction and may have equal flexibility in all radial directions. Such a feature may allow the hypotube 700 to have a greater variety of orientations about the directions of bend of the rail assembly, as the hypotube 700 lacks a particular bias towards one direction of bend of the rail assembly. Further, the circumferential length and longitudinal width of the cuts 702a-d is equal in FIG. 18, with equal offset of cuts in repeating rows of cuts.

Further, the small width of the cuts 702a-d of the hypotube 700 may allow the plurality of rings 704a-b to contact and compress against each other to transmit a distal force for implant recapture or the like. The gaps formed by the cuts 702a-d may be reduced in size when the hypotube 700 is distally compressed, such that a rigid rod structure is formed for recapture of the implant. The lack of bias of the hypotube 700 may also reduce the possibility of the hypotube 700 deflecting in a direction due to a flex bias of the hypotube 700.

FIG. 19 illustrates an embodiment of a hypotube 800 shown in a flattened configuration that may be utilized with assemblies of the present disclosure. The hypotube 800 may be utilized as a metal layer, or metal portion of one or more of the assemblies disclosed herein, and particularly may comprise a portion of a capsule 106 as shown in FIG. 4. The hypotube 800 may be utilized with a capsule 106 that is configured to surround an implant retention area 16 of the elongate shaft 12. The hypotube 800 may be utilized as a metal layer 404 for a capsule as shown in FIG. 15, and may be utilized in other portions of the assemblies that utilize hypotubes or other portions of the assemblies. The hypotube 800 may be positioned between an outer jacket and a liner layer, among other components disclosed in embodiments herein. Although shown in a flattened configuration in FIG. 19, the hypotube 800 has outer sides 804, 806 that when connected form a cylindrical shape for the hypotube 800 as shown in FIG. 19.

The hypotube 800 is configured similarly as the hypotube 700 shown in FIG. 18, however, the size of the one or more cuts 802a-d that form a plurality of rings 806a-b of the hypotube has increased. The length and width of the cuts 802a-d remains equal, however. The increased size of the cuts 802a-d may allow for greater flexibility of the hypotube 800. The size of the cuts 802a-d may be reduced when the hypotube 800 is distally compressed, such that a rigid rod structure is formed for recapture of the implant. The lack of bias of the hypotube 800 may also reduce the possibility of the hypotube 800 deflecting in a direction due to a flex bias of the hypotube 800.

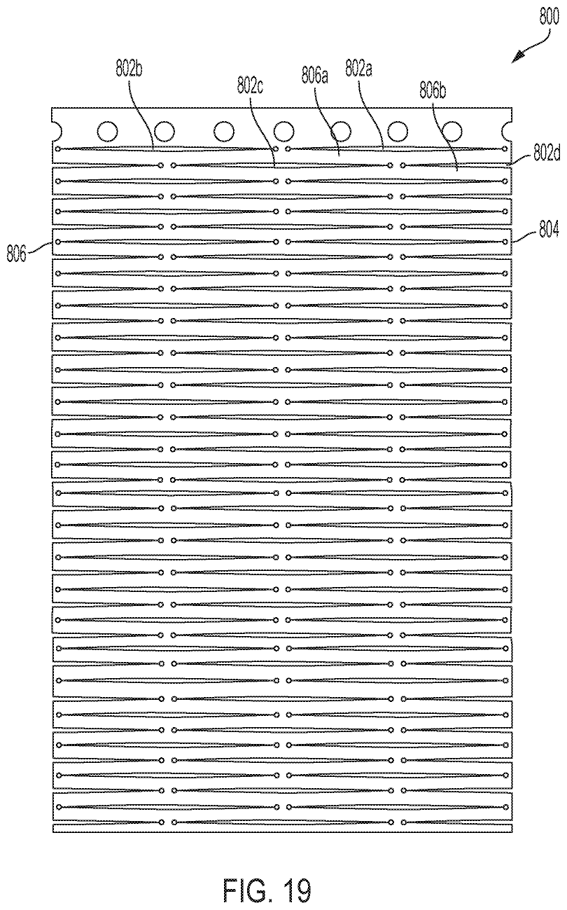

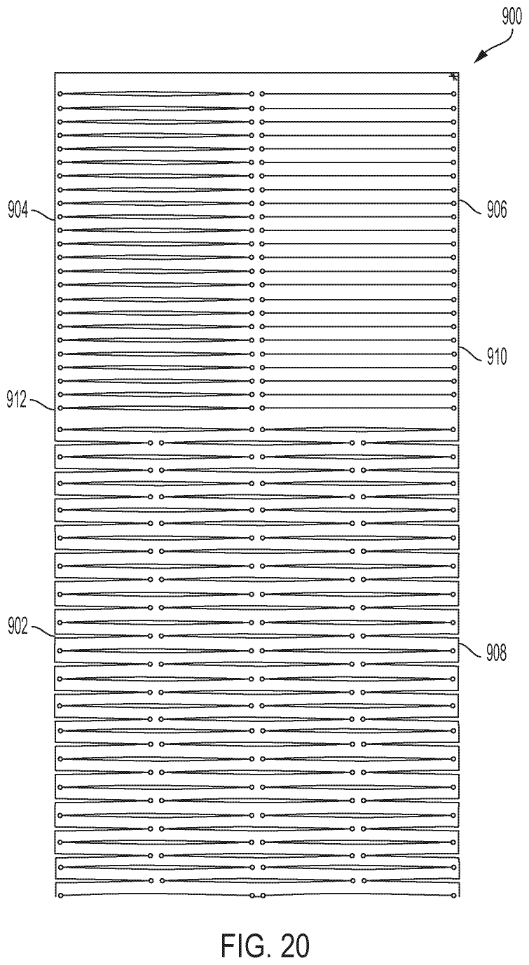

In certain embodiments, a hypotube may be utilized that includes a combination of a cut pattern having a bias to flex in a direction, and a cut pattern lacking a bias to flex in a direction. FIG. 20 illustrates such a pattern, in which a hypotube 900 includes a first section 902 that has a cut pattern 908 that is configured as the cut pattern of the hypotube 800 shown in FIG. 19. The hypotube 900 includes a second section 904 with a cut pattern 906 that is configured as the cut pattern of the section 506 of the hypotube 500 shown in FIG. 16. The first section 902 may be positioned proximally and the second section 904 may be positioned distally.

The hypotube 900 is shown in a flattened configuration that may be utilized with assemblies of the present disclosure. The hypotube 900 may be utilized as a metal layer, or metal portion of one or more of the assemblies disclosed herein, and particularly may comprise a portion of a capsule 106 as shown in FIG. 4. The hypotube 900 may be utilized with a capsule 106 that is configured to surround an implant retention area 16 of the elongate shaft 12. The hypotube 900 may be utilized as a metal layer 404 for a capsule as shown in FIG. 15, and may be utilized in other portions of the assemblies that utilize hypotubes or other portions of the assemblies. The hypotube 900 may be positioned between an outer jacket and a liner layer, among other components disclosed in embodiments herein. Although shown in a flattened configuration in FIG. 20, the hypotube 900 has outer sides 910, 912 that when connected form a cylindrical shape for the hypotube 900 as shown in FIG. 20.

The hypotubes 600, 700, and 800 and the cut pattern 908 shown in FIG. 20 may beneficially lack a bias of towards a direction of bend, yet may provide flexibility in multiple directions of bend. The hypotubes 600, 700, and 800 and the cut pattern 908 may also provide a compressive force that allows the hypotubes 600, 700, and 800 and the cut pattern 908 to be utilized for implant recapture if desired.

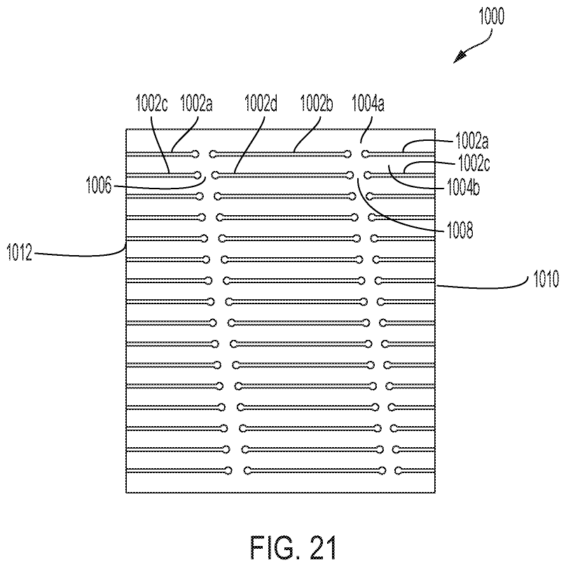

FIG. 21 illustrates an embodiment of a hypotube 1000 shown in a flattened configuration that may be utilized with assemblies of the present disclosure. The hypotube 1000 may be utilized as a metal layer, or metal portion of one or more of the assemblies disclosed herein, and particularly may comprise a portion of a capsule 106 as shown in FIG. 4. The hypotube 1000 may be utilized with a capsule 106 that is configured to surround an implant retention area 16 of the elongate shaft 12. The hypotube 1000 may be utilized as a metal layer 404 for a capsule as shown in FIG. 15, and may be utilized in other portions of the assemblies that utilize hypotubes or other portions of the assemblies. The hypotube 1000 may be positioned between an outer jacket and a liner layer, among other components disclosed in embodiments herein. Although shown in a flattened configuration in FIG. 21, the hypotube 1000 has outer sides 1010, 1012 that when connected form a cylindrical shape for the hypotube 1000 as shown in FIG. 21.

Pairs of cuts (one pair includes cuts 1002a, b and another pair includes cuts 1002c, d) may be offset circumferentially from each other such that the spines 1006, 1008 form an angle relative to the longitudinal direction. The cuts 1002a, b and 1002c, d accordingly may form an angled pattern or sweep pattern in which the hypotube 1000 has a bias to flex in a direction with the spines 1006, 1008 extending along the neutral axis of bend. The direction of bend bias accordingly may vary along the length of the hypotube 1000. The gaps formed by the cuts 1002a, b and 1002c, d may be reduced in size when the hypotube 1000 is distally compressed, such that a rigid rod structure is formed for recapture of the implant.

Notably, the directions of bias of the bend of the hypotube 1000 should be aligned with the directions of bend of the rail assembly. It may thus beneficial to allow a portion of the delivery system including a hypotube having a biased direction of flex, to rotate to align the bias direction with the direction of bend of a rail assembly, or another structure forming a bend of the delivery system. The embodiments of hypotubes disclosed herein may be utilized solely, or may be utilized with any of the other apparatuses, systems, or methods disclosed herein.

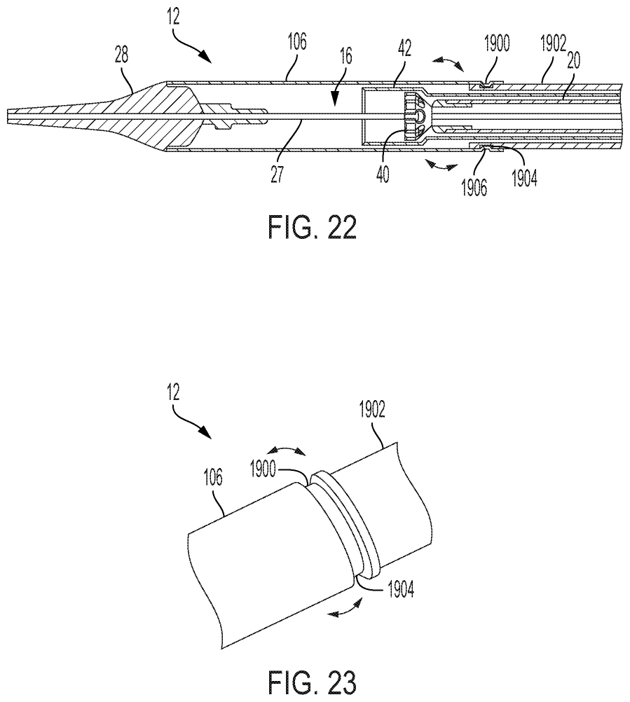

FIG. 22-25 illustrate embodiments in which the delivery system 10 may be configured to allow the capsule 106 to rotate. The rotation of the capsule 106 may allow a hypotube included with the capsule 106 to rotate such that a direction of bias of a cut pattern of the hypotube aligns with a direction of bend of the rail assembly. The capsule 106 may be configured to surround the implant retention area 16. Thus, for the embodiments of hypotubes 500, 900, 1000 that include a direction of flex bias, the capsule 106 may be able to rotate to align the direction of the flex bias with a direction of bend of the rail assembly. Such a feature may be useful to reduce the possibility of hypotubes that include a direction of flex bias from becoming misaligned with the direction of bend of the rail assembly and having reduced operability.

FIG. 22 illustrates a distal portion of a delivery system that includes a coupler 1900 configured to couple the capsule 106 to a shaft portion 1902 of the elongate shaft 12 that is positioned proximal of the capsule 106. The shaft portion 1902 may comprise a portion of the outer shaft assembly, which may include the outer hypotube 104, or the shaft 102, or another portion of the outer shaft assembly as desired.

The coupler 1900 may allow the capsule 106 to rotate about the axis of the elongate shaft 12. The coupler 1900 may take a variety of forms and may include a protrusion 1904 that is positioned within a channel 1906. The protrusion 1904 may be configured to rotate relative to the channel 1906 to allow the capsule 106 to rotate. As shown in FIG. 22, the protrusion 1904 may comprise a material that is deflected into the channel 1906. For example, the protrusion 1904 may be swaged into the channel 1906 and thus able to rotate relative to the channel 1906. The protrusion 1904 may be coupled to the capsule 106, such as a proximal portion of the capsule 106, and the channel 1906 may be coupled to a distal portion of the shaft portion 1902. In other embodiments, the protrusion 1904 may be coupled to the shaft portion 1902 and the channel 1906 may be coupled to the capsule 106. The protrusion 1904 and the channel 1906 may be positioned in other locations on either the capsule 106 or the shaft portion 1902 as desired.

FIG. 23 illustrates a side perspective view of an exterior of the elongate shaft 12 including the coupler 1900. The capsule 106 may be configured to rotate about the axis of the elongate shaft 12 relative to the shaft portion 1902, as indicated with the arrows shown in FIG. 23.

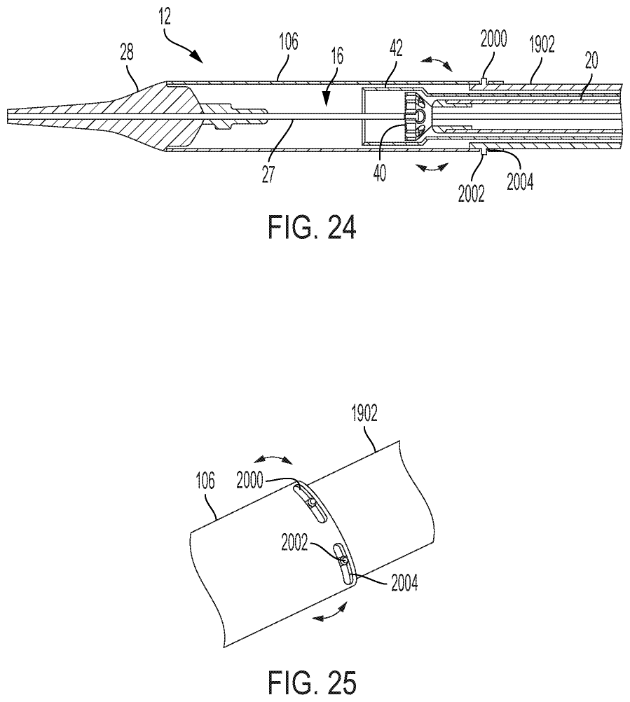

FIG. 24 illustrates an embodiment of the coupler 2000 in which the coupler comprises a protrusion 2002 that may be configured to rotate relative to the channel 2004 to allow the capsule 106 to rotate. As shown in FIG. 24, the protrusion 2002 may comprise a pin. The channel 2004 may comprise a window. The pin may be configured to slide along the window to allow the capsule 106 to rotate. The window may be coupled to the capsule 106, such as a proximal portion of the capsule 106, and the pin may be coupled to a distal portion of the shaft portion 1902. In other embodiments, the window may be coupled to the shaft portion 1902 and the pin may be coupled to the capsule 106. The protrusion 2002 and the channel 2004 may be positioned in other locations on either the capsule 106 or the shaft portion 1902 as desired.

FIG. 25 illustrates a side perspective view of an exterior of the elongate shaft 12 including the coupler 2000. The capsule 106 may be configured to rotate about the axis of the elongate shaft 12 relative to the shaft portion 1902, as indicated with the arrows shown in FIG. 25.

The couplers 1900, 2000 shown in FIG. 22-25 may take a variety of other forms as desired, and may be varied from the protrusion and channel configurations shown in FIG. 22-25. The perspective views shown in FIGS. 23 and 25 show the couplers positioned on an outer surface of the elongate shaft 12, however, in other embodiments the couplers may be covered by an outer jacket or another structure as desired.

The couplers 1900, 2000 may beneficially allow the capsule 106 to rotate. The rotation of the capsule 106 may allow a hypotube included with the capsule 106 to rotate such that a direction of bias of a cut pattern of the hypotube aligns with a direction of bend of the rail assembly. Thus, for the embodiments of hypotubes 500, 900, 1000 that include a direction of flex bias, the capsule 106 may be able to rotate to align the direction of the flex bias with a direction of bend of the rail assembly.

The hypotubes 500, 900, 1000 may be configured to passively rotate to align the direction of the flex bias with a direction of bend of the rail assembly. As such, as the hypotubes 500, 900, 1000 pass over the bend of the rail assembly, the reduced resistance to bending caused by aligning the direction of the flex bias with a direction of bend of the rail assembly, may allow the hypotubes 500, 900, 1000 to passively rotate.

Although the couplers 1900, 2000 are shown in relation to the capsule 106, in other embodiments other portions of the elongate shaft 12 may be configured to rotate through use of the couplers 1900, 2000. Such portions may comprise a mid shaft assembly, which may include an outer retention ring 42 that may be configured to rotate in a similar manner as the capsule 106. The mid shaft assembly may include a hypotube, of which a coupler may allow the outer retention ring 42 to rotate with such that a direction of bias of a cut pattern of the hypotube aligns with a direction of bend of the rail assembly. Other portions of the elongate shaft 12 may utilized couplers for rotation as desired.

The rotation of the portion of the elongate shaft 12, which may include the capsule 106 may allow for rotation during a proximal retraction or a distal advancement of the portion of the elongate shaft 12. For example, the capsule 106 may rotate via one of the couplers 1900, 2000 when the capsule is being retracted proximally, or may rotate via one of the couplers 1900, 2000 when the capsule if being advanced distally. Notably, during advancement of the capsule 106 distally, a compressive force may be applied to the capsule 106 both in a proximal direction due to compression of the expandable implant, and in a distal direction due to the force applied to the capsule 106 by the handle of the delivery system. It is noted that as a result of such forces, structural damage may be produced, such as crushing of the capsule 106 if such forces are sufficiently strong. The embodiments of couplers and capsules disclosed herein may be utilized solely, or may be utilized with any of the other apparatuses, systems, or methods disclosed herein.

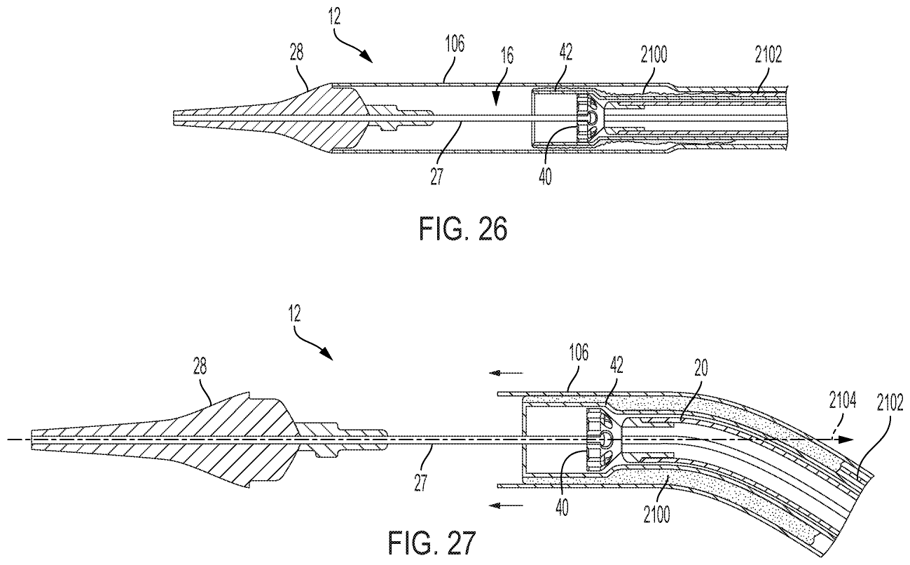

FIG. 26 illustrates a distal portion of a delivery system that includes an inflatable body 2100. The inflatable body 2100 may be configured to inflate to support a sheath. The inflation of the inflatable body 2100 may serve to reduce the possibility of structural damage to the sheath and may strengthen the sheath during distal movement such as implant recapture.

As shown in FIG. 26, the sheath may comprise an outer sheath, which may include the capsule 106 and other portions such as an outer hypotube 104 and a shaft 102 (as shown in FIG. 4). The inflatable body 2100 may be configured to inflate to support the outer shaft when the outer shaft is being extended distally. For example, when the capsule 106 is being extended distally for recapture of an expandable implant, the inflatable body 2100 may be inflated to provide support for the capsule 106 and other portions of the sheath. The support may reduce the possibility of structural damage to the capsule 106, as structural strength is provided to the capsule 106.

As shown in FIG. 26, the inflatable body 2100 may be positioned within an interior lumen of the sheath. The inflatable body 2100 may be positioned on an interior shaft that may be positioned within the lumen of the sheath. The inflatable body 2100 may be positioned between the interior shaft and the sheath and configured to inflate to support the sheath. The interior shaft may comprise a middle shaft (mid shaft) of the elongate shaft 12 as shown, or in other embodiments may comprise another shaft within the elongate shaft 12. The inflatable body 2100 may be positioned on a shaft that is adjacent to the sheath, such that inflation of the inflatable body 2100 causes contact of an interior surface of the sheath.

A conduit 2102 may couple to the inflatable body 2100 and be configured to provide fluid to inflate the inflatable body 2100. The conduit 2102 may extend along the length of the elongate shaft 12 and may be positioned within the outer lumen of the outer shaft. A proximal end of the conduit 2102 may couple to an inflation port or the like for inflating the inflatable body 2100. The conduit 2102 may extend between the outer sheath and the mid sheath as shown in FIG. 26. In other embodiments, the conduit 2102 may be positioned in other locations as desired.

The inflatable body 2100 may include one or more balloons, which may be pleated or may have another form. The inflatable body 2100 may have other forms in other embodiments. The inflatable body 2100 may be configured to exert sufficient force against the interior surface of the sheath such that the sheath is supported upon the inflatable body 2100 inflating and contacting the interior surface of the sheath. Such support may occur as the sheath is being advanced distally, and may occur as the sheath is bent around the rail assembly 20.

FIG. 27 illustrates the inflatable body 2100 inflated and supporting the sheath (shown as the capsule 106 of the outer sheath) at a bend of the outer shaft. The capsule 106 is being advanced distally, which may be for recapture of an expandable implant (although the implant is not visible in FIG. 27). A resistive force in the direction 2104 marked with a dashed arrow in FIG. 27 may be applied to the capsule 106. Without the presence of the inflatable body 2100, the resistive force in the direction 2104 may damage the capsule 106. The inflatable body 2100, however, may inflate to contact the interior surface of the capsule 106, and fill the space between the capsule 106 and the mid shaft. The inflatable body 2100 may then be deflated at a desired time.

Although the outer sheath is shown as the capsule 106 in FIG. 27, an inflatable body may be utilized in any embodiment of sheath. For example, the mid shaft may comprise a sheath that extends around the inner shaft, with the inner shaft positioned within the outer sheath of the mid shaft. An inflatable body may be positioned between the inner shaft and the sheath of the mid shaft and may support the mid shaft as the mid shaft is advanced proximally for recapture of an implant or the like. The inflatable body may be utilized in other locations as desired. The embodiments of inflatable bodies and shafts disclosed herein may be utilized solely, or may be utilized with any of the other apparatuses, systems, or methods disclosed herein.

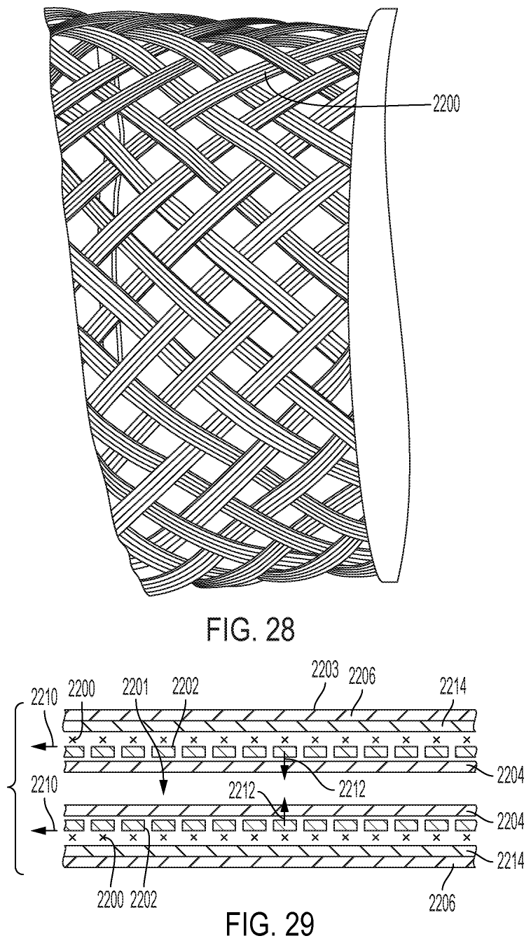

In certain embodiments, the elongate shaft 12 of the delivery system may include a wall having a tensile layer that provides strength to the elongate shaft 12 upon being withdrawn proximally. FIG. 28, for example, illustrates an embodiment of a tensile layer in the form of a braid layer 2200 that may be utilized to provide strength to the elongate shaft 12 upon being withdrawn proximally.

The braid layer 2200 may comprise a weave, as shown in FIG. 28, of overlapping metal wires or other forms of fibers, that form a sheath. In other embodiments, other materials may be utilized to form the braid layer 2200 as desired.

FIG. 29 illustrates a cross sectional view of a wall of a sheath of the elongate shaft including the braid layer of FIG. 28. A wall 2203 extends circumferentially around an interior lumen 2201 to form the body of the sheath. The construction of the wall 2203 of the sheath is visible, including the braid layer 2200 surrounding a metal layer 2202. An interior liner layer 2204 is visible surrounded by the metal layer 2202. An outer jacket layer 2206 is visible extending around the braid layer 2200. An interior lumen 2201 is surrounded by the sheath.

The metal layer 2202 may be configured similarly as the metal layers disclosed within this application, including embodiments of a coil or a hypotube disclosed herein, including the hypotubes shown in FIG. 16-21. The braid layer 2200 may surround the metal layer 2202 such that as an axial force 2210 providing tension is applied to the braid layer 2200, the braid layer 2200 compresses onto the metal layer 2202. The compression is represented by arrows 2212. The metal layer 2202 may resist the compression of the braid layer 2200 upon the metal layer 2202, thus causing the braid layer 2200 to exhibit a high tensile strength. The braid layer 2200 may thus be utilized to strengthen the sheath of the elongate shaft, allowing a greater retraction force to be applied by the sheath of the elongate shaft.

A buffer layer 2214 may be positioned between the outer jacket layer 2206 and the braid layer 2200. The buffer layer 2214 may prevent the outer jacket layer 2206 from flowing between the weave of the braid layer 2200 and thus diminishing the effectiveness of the braid layer 2200 by filling the interstices of the braid layer 2200 with material. The buffer layer 2214 may be made of a polymer and may comprise expanded polytetrafluoroethylene (ePTFE) or in other embodiments may comprise other materials. The outer jacket layer 2206 may comprise PEBAX or another form of polymer.

The construction of the wall of the sheath of the elongate shaft as shown in FIG. 29, including the use of the braid layer 2200 may be utilized with various assemblies of the delivery system. For example, the capsule 106 of the outer sheath may be configured to include the construction shown in FIG. 29. In other embodiments, other assemblies, including any portion of the outer sheath, a middle sheath, or the inner assembly may utilize the construction of the elongate shaft as shown in FIG. 29. The construction of the wall of the sheath disclosed herein may be utilized solely, or may be utilized with any of the other apparatuses, systems, or methods disclosed herein.

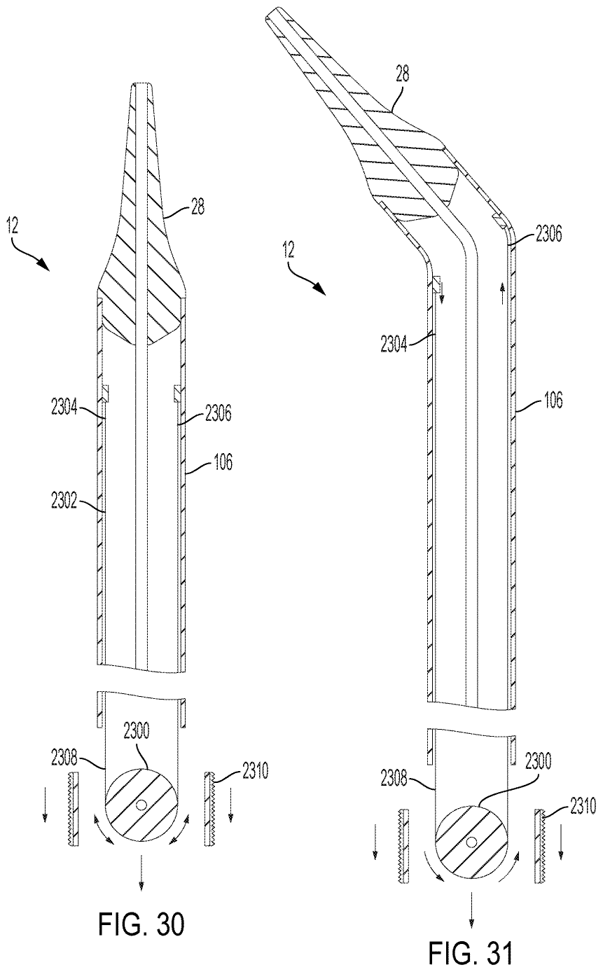

Other devices, solely or in combination with a braid layer, may improve the retraction force of a portion of the elongate sheath. FIG. 30 illustrates an embodiment of a delivery system that utilizes a cable router 2300. The delivery system may utilize a cable 2302 having two end portions 2304, 2306. The end portions 2304, 2306 may couple to a sheath, which may comprise the capsule 106 as shown in FIG. 30, but in other embodiments may comprise other sheaths or portions of the delivery system, including the elongate shaft. The cable 2302 may include an intermediate portion 2308 that may extend between the end portions 2304, 2306. The end portions 2304, 2306 may be coupled to respective opposite sides of the sheath. For example, the end portion 2304 may couple to an interior surface of the sheath and the end portion 2306 may couple to an interior surface of the sheath on an opposite side of the sheath. The end portions 2304, 2306 may be separated by the interior lumen of the sheath. The end portions 2304, 2306 may couple to portions of the sheath that are positioned on opposite sides of a neutral axis of the sheath within the same plane of bend. For example, the end portion 2304 may couple to a portion of the sheath that forms an interior curvature of the sheath when the sheath is bent, and the end portion 2306 may couple to a portion of the sheath that forms an outer curvature of the sheath when the sheath is bent. If the sheath is bent in the opposite direction, then the end portion 2304 may couple to a portion of a sheath that forms an outer curvature of the sheath, and the end portion 2306 may couple to a portion of the sheath that forms an inner curvature of the sheath when the sheath is bent in the opposite direction.

The cable 2302 may be configured to slide along the length of the sheath, and may extend within the interior lumen of the sheath. The cable 2302 may extend along the length of the sheath to engage the cable router 2300.

The cable router 2300 may take a variety of forms and may comprise a pulley wheel as shown in FIG. 30. The cable router 2300 may comprise a channel to allow the cable 2302 to bend around the cable router 2300. The channel for example may comprise a bent tube that redirects the direction of the cable 2302. Other forms of cable router 2300 may be utilized as desired.

The cable router 2300 may be configured to engage the intermediate portion 2308 of the cable 2302 such that the cable may move along the cable router 2300 when the cable 2302 is slid due to a bending of the sheath such as the capsule 106 shown in FIG. 30. For example, in an embodiment in which the cable router 2300 is a pulley wheel, the pulley wheel may rotate to allow the cable 2302 to move along the cable router 2300. In an embodiment in which the cable router 2300 is a channel, the channel may allow the cable 2302 to slide along the channel.

The delivery system may include a control mechanism 2310. The control mechanism 2310 may comprise a body configured to move the cable router 2300 by applying tension to the cable router 2300 to retract the cable router 2300 in a proximal direction. The control mechanism 2310 may be configured to retract the sheath as well in a similar manner. The control mechanism 2310 may include threading or the like to allow the control mechanism 2310 to be operated by a mechanism of the handle or other portion of the delivery system. The cable router 2300 and control mechanism 2310 may be positioned in a handle of a delivery system or in another location as desired.

In operation, the cable 2302 may be configured to passively have its length vary between the end portion 2304 and the cable router 2300, and between the end portion 2306 and the cable router 2300, due to a deflection of the sheath. FIG. 31, for example, illustrates an operation of the cable 2302 and cable router 2300. The sheath, shown as capsule 106, may be deflected due to a variety of reasons, which may include a deflection of the rail assembly. The deflection of the sheath causes the end portion 2304 of the cable 2302 to be moved proximally, and the end portion 2306 of the cable 2302 to be moved distally. The amount of proximal movement of the end portion 2304 of the cable 2302 (the change in length) may be equal to the amount of distal movement of the end portion 2306 of the cable 2302 (the change in length) and the variation in length may simultaneously occur. The cable router 2300 may serve to allow the intermediate portion 2308 of the cable 2302 to move along the cable router 2300 to transfer the length of the cable 2302 from the end portion 2304 to the end portion 2306. Alternatively, the sheath may be deflected in an opposite direction, with the end portion 2304 extending distally and the end portion 2306 extending proximally.

The length of the cable 2302 may be routed between the end portions 2304, 2306 to allow the cable 2302 to remain taut between the end portions 2304, 2306 during flex of the sheath. The control mechanism 2310 may retract the cable router 2300 to allow the cable 2302 to pull on the sheath to enhance a pull strength of the sheath if the sheath is retracted. Such a feature may be utilized with a capsule 106 that may be retracted to allow an expandable implant to be deployed. Such a feature may be utilized with any other portion of the elongate sheath that is retracted, such as a middle sheath that is retracted to retract an outer retention ring 42. A sheath coupled to a nose cone of the elongate shaft may be retracted as well, among other locations of use. The cable router disclosed herein may be utilized solely, or may be utilized with any of the other apparatuses, systems, or methods disclosed herein.

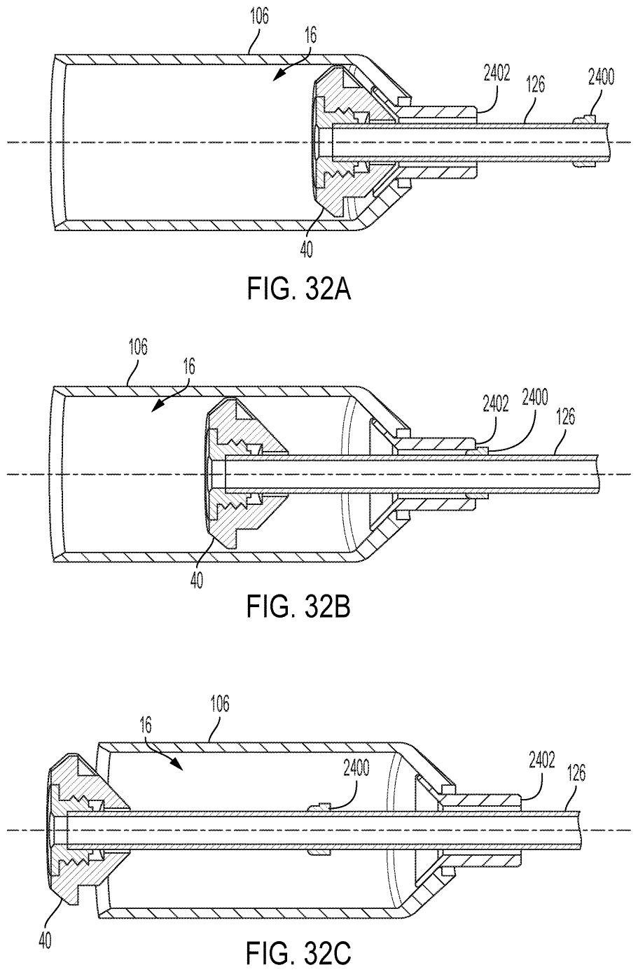

FIGS. 32-34 illustrate distal portions of delivery systems including stops that are positioned on an interior shaft of the delivery system and configured to impede proximal movement of the capsule 106. FIG. 32A illustrates a side cross sectional view of a distal portion of the delivery system. The capsule 106 is shown to extend over the inner sheath assembly in FIG. 32A, although in other embodiments the capsule 106 may extend over other interior shafts such as the mid shaft assembly.

The interior shaft, shown as the distal section 126 of the inner shaft assembly, may include a stop 2400 positioned thereon. The stop 2400 may comprise a protrusion that extends radially outward from the interior shaft. The stop 2400 may be configured such that the stop 2400 applies a force to a proximal body 2402 of the capsule 106 that impedes proximal movement of the capsule 106. FIG. 32B, for example, illustrates the capsule 106 having been withdrawn, with the proximal body 2402 contacting the stop 2400. The position of the capsule 106 may thus be held until additional force is applied to the capsule 106 to overcome the protrusion of the stop 2400 and allow the capsule 106 to further move proximally to allow the expandable implant to deploy from the implant retention area 16. FIG. 32C, for example, illustrates the capsule 106 having been withdrawn proximally past the stop 2400.

The stop may have a variety of other forms as desired. For example, in FIGS. 33A-C, the stop 2404 may have the form of threading on the interior shaft. The capsule may include a threaded portion 2406 that may allow the capsule 106 to be rotated relative to the threading on the interior shaft to allow the capsule 106 to move proximally past the stop 2404. FIG. 33C, for example, illustrates the capsule 106 having been withdrawn proximally past the stop 2404.

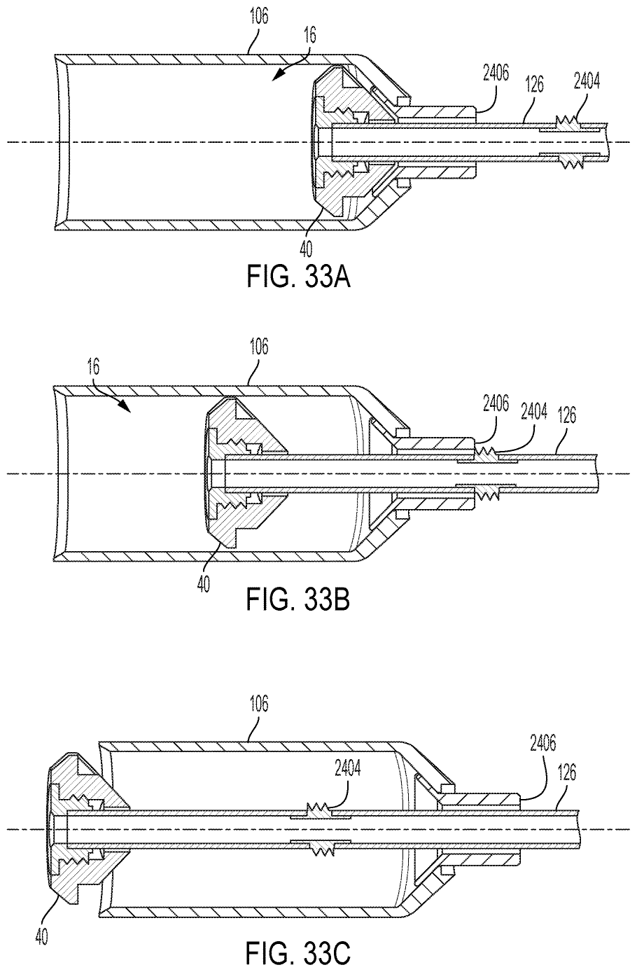

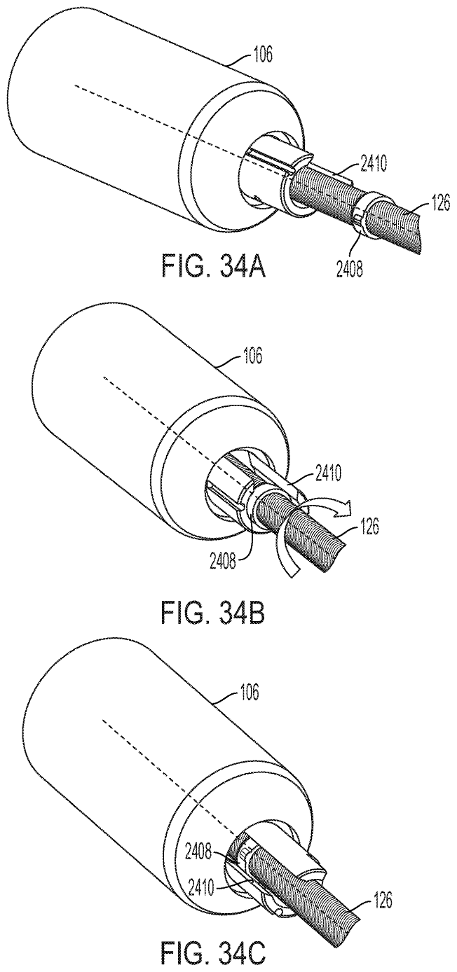

In FIGS. 34A-C, the stop 2408 may have the form of a keyed shape on the interior shaft. The capsule 106 may include complementary keyed shape on a proximal body 2410 that is matched by the stop 2408 to allow the capsule 106 to move proximally past the stop. The capsule 106 may be rotated relative to the threading on the interior shaft to allow the capsule 106 to move proximally past the stop 2408. FIG. 34C, for example, illustrates the capsule 106 having been withdrawn proximally past the stop 2408.

The stops shown in FIGS. 32-34 may be configured to provide for two stage deployment of the implant retained within the implant retention area 16. The stops may be positioned on the interior shaft to allow the capsule 106 to retract for a defined distance, to allow a defined amount of partial release, or partial exposure, or partial expansion of the implant upon the stop being contacted. For example, as shown in FIG. 33B, the capsule 106 may be retracted for a defined distance until the stop 2400 is contacted. The defined distance may be a stopping point for the user of the delivery system, to assure at this point that the implant is partially deployed in the desired location. The stop 2400 may prevent the user from retracting the capsule 106 entirely, without first stopping to confirm that the expandable implant is partially deployed in the desired location. Upon the user performing such a confirmation, the capsule 106 may continue to be retracted past the stop 2400 to allow the expansion and deployment of the expandable implant to continue. The stop 2400 thus may serve as a tactile feedback for the user of a desired stop location for the user to assure at this point that the expandable implant is partially deployed in the desired location. The stops 2404, 2408 may serve a similar function as stop 2400. The stops may be positioned proximate the capsule 106, to allow for tactile feedback that is positioned proximate the capsule 106, which may better allow the user to operate the delivery system. After the stops are overcome, full release or exposure or expansion of the implant may occur.

The stops may have varied configurations and positions than shown in FIGS. 32-34, as desired. The stops may be positioned on an interior shaft such as a middle shaft (mid shaft) or an inner shaft as desired, among other locations. The stops disclosed herein may be utilized solely, or may be utilized with any of the other apparatuses, systems, or methods disclosed herein.

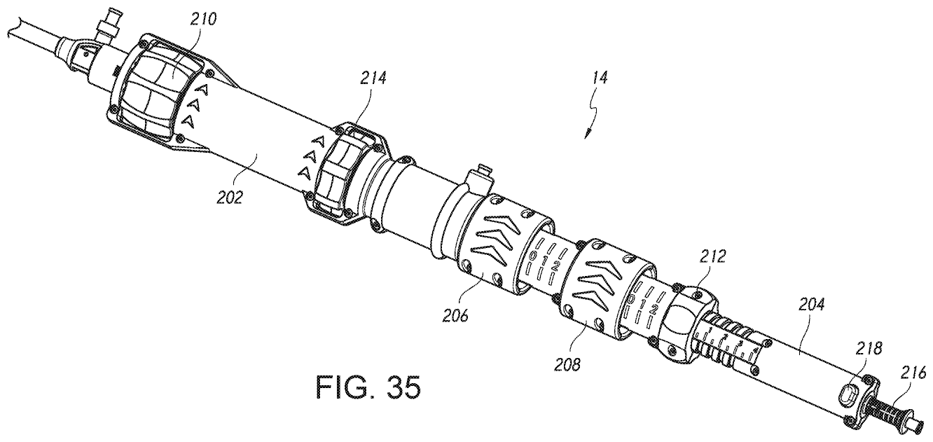

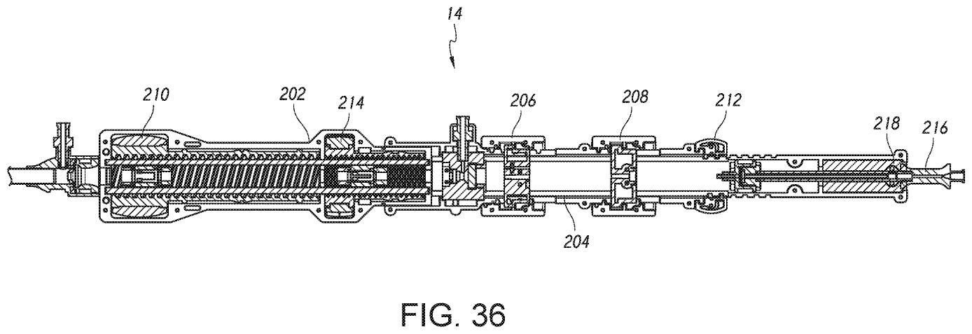

Referring to FIG. 35, a handle 14 is located at the proximal end of the delivery system 10. A cross-section of the handle 14 is shown in FIG. 36. The handle 14 can include a number of actuators, such as rotatable knobs, that can manipulate different components of the delivery system 10. The operation of the handle 14 is described with reference to delivery of a replacement mitral valve implant 70, though the handle 14 and delivery system 10 can be used to deliver other devices as well.

The handle 14 is generally composed of two housings, a rail housing 202 and a delivery housing 204, the rail housing 202 being circumferentially disposed around the delivery housing 204. The inner surface of the rail housing 202 can include a screwable section configured to mate with an outer surface of the delivery housing 204. Thus, the delivery housing 204 is configured to slide (e.g., screw) within the rail housing 202, as detailed below. The rail housing 202 generally surrounds about one half the length of the delivery housing 204, and thus the delivery housing 204 extends both proximally and distally outside of the rail housing 202.

The rail housing 202 can contain two rotatable knobs, a distal pull wire knob 206 and a proximal pull wire knob 208. However, the number of rotatable knobs on the rail housing 202 can vary depending on the number of pull wires used. Rotation of the distal pull wire knob 206 can provide a proximal force, thereby providing axial tension on the distal pull wires 138 and causing the distal slotted section 235 of the rail hypotube 136 to bend. The distal pull wire knob 206 can be rotated in either direction, allowing for bending in either direction, which can control anterior-posterior angles. Rotation of the proximal pull wire knob 208 can provide a proximal force, and thus axial tension, on the proximal pull wires 140, thereby causing the proximal slotted section 233 of the rail hypotube 136 to bend, which can control the medial-lateral angle. The proximal pull wire knob 208 can be rotated in either direction, allowing for bending in either direction. Thus, when both knobs are actuated, there can be two bends in the rail hypotube 136, thereby allowing for three-dimensional steering of the rail shaft 132, and thus the distal end of the delivery system 10. Further, the proximal end of the rail shaft 132 is connected on an internal surface of the rail housing 202.

The bending of the rail shaft 132 can be used to position the system, in particular the distal end, at the desired patient location, such as at the native mitral valve. In some embodiments, rotation of the pull wire knobs 206/208 can help steer the distal end of the delivery system 10 through the septum and left atrium and into the left ventricle so that the implant 70 is located at the native mitral valve.

Moving to the delivery housing 204, the proximal ends of the inner shaft assembly 18, outer sheath assembly 22, mid shaft assembly 21, and nose cone shaft assembly 31 can be connected to an inner surface of the delivery housing 204 of the handle 14. Thus, they can move axially relative to the rail assembly 20 and rail housing 202.

A rotatable outer sheath knob 210 can be located on the distal end of the delivery housing 204, being distal to the rail housing 202. Rotation of the outer sheath knob 210 will pull the outer sheath assembly 22 in an axial direction proximally, thus pulling the capsule 106 away from the implant 70 and releasing the distal end 303 of implant 70. Thus the outer sheath assembly 22 is individually translated with respect to the other shafts in the delivery system 10. The distal end 303 of the implant 70 can be released first, while the proximal end 301 of the implant 70 can remain radially compressed between the inner retention member 40 and the outer retention member 42.

A rotatable mid shaft knob 214 can be located on the delivery housing 204, in some embodiments proximal to the rotatable outer sheath knob 210, being distal to the rail housing 202. Rotation of the mid shaft knob 214 will pull the mid shaft assembly 21 in an axial direction proximally, thus pulling the outer retention ring 42 away from the implant 70 and uncovering the inner retention member 40 and the proximal end 301 of the implant 70, thereby releasing the implant 70. Thus, the mid shaft assembly 21 is individually translated with respect to the other shafts in the delivery system 10.

Located on the proximal end of the delivery housing 204, and thus proximal to the rail housing 202, can be a rotatable depth knob 212. As the depth knob 212 is rotated, the entirety of the delivery housing 204 moves distally or proximally with respect to the rail housing 202 which will remain in the same location. Thus, at the distal end of the delivery system 10, the inner shaft assembly 18, outer sheath assembly 22, mid shaft assembly 21, and nose cone shaft assembly 31 together (e.g., simultaneously) move proximally or distally with respect to the rail assembly 20 while the implant 70 remains in the compressed configuration. In some embodiments, actuation of the depth knob 212 can sequentially move the inner shaft assembly 18, outer sheath assembly 22, mid shaft assembly 21, and nose cone shaft assembly 31 relative to the rail assembly 20. In some embodiments, actuation of the depth knob 212 can together move the inner shaft assembly 18, outer sheath assembly 22, and mid shaft assembly 21 relative to the rail assembly 20. Accordingly, the rail shaft 132 can be aligned at a particular direction, and the other assemblies can move distally or proximally with respect to the rail shaft 132 for final positioning while not releasing the implant 70. The components can be advanced approximately 1, 2, 3, 5, 6, 7, 8, 9, or 10 cm along the rail shaft 132. The components can be advanced more than approximately 1, 2, 3, 5, 6, 7, 8, 9, or 10 cm along the rail shaft 132. An example of this is shown in FIG. 2C. The capsule 106 and outer retention ring 42 can then be individually withdrawn with respect to the inner assembly 18 as discussed above, in some embodiments sequentially, releasing the implant 70. The assemblies other than the rail assembly 20 can then be withdrawn back over the rail shaft 132 by rotating the depth knob 212 in the opposite direction.

The handle 14 can further include a mechanism (knob, button, handle) 216 for moving the nose cone shaft 27, and thus the nose cone 28. For example, a knob 216 can be a portion of the nose cone assembly 31 that extends from a proximal end of the handle 14. Thus, a user can pull or push on the knob 216 to translate the nose cone shaft 27 distally or proximally individually with respect to the other shafts. This can be advantageous for proximally translating the nose cone 28 into the outer sheath assembly 22/capsule 106, thus facilitating withdraw of the delivery system 10 from the patient.

In some embodiments, the handle 14 can provide a lock 218, such as a spring lock, for preventing translation of the nose cone shaft 27 by the knob 216 discussed above. In some embodiments, the lock 218 can be always active, and thus the nose cone shaft 27 will not move without a user disengaging the lock 218. The lock can be, for example, a spring lock that is always engaged until a button 218 on the handle 14 is pressed, thereby releasing the spring lock and allowing the nose cone shaft 27 to translate proximally/distally. In some embodiments, the spring lock 218 allows one-way motion, either proximal or distal motion, of the nose cone shaft 27 but prevents motion in the opposite direction.

The handle 14 can further include a communicative flush port for flushing out different lumens of the delivery system 10. In some embodiments, a single flush port on the handle 14 can provide fluid connection to multiple assemblies. In some embodiments, the flush port can provide fluid connection to the outer sheath assembly 22. In some embodiments, the flush port can provide fluid connection to the outer sheath assembly 22 and the mid shaft assembly 21. In some embodiments, the flush port can provide fluid connection to the outer sheath assembly 22, the mid shaft assembly 21, and the rail assembly 20. In some embodiments, the flush port can provide fluid connection to the outer sheath assembly 22, the mid shaft assembly 21, the rail assembly 20, and the inner assembly 18. Thus, in some embodiments, the rail shaft 132, the outer retention ring 42, and the capsule 106 can all be flushed by a single flush port.

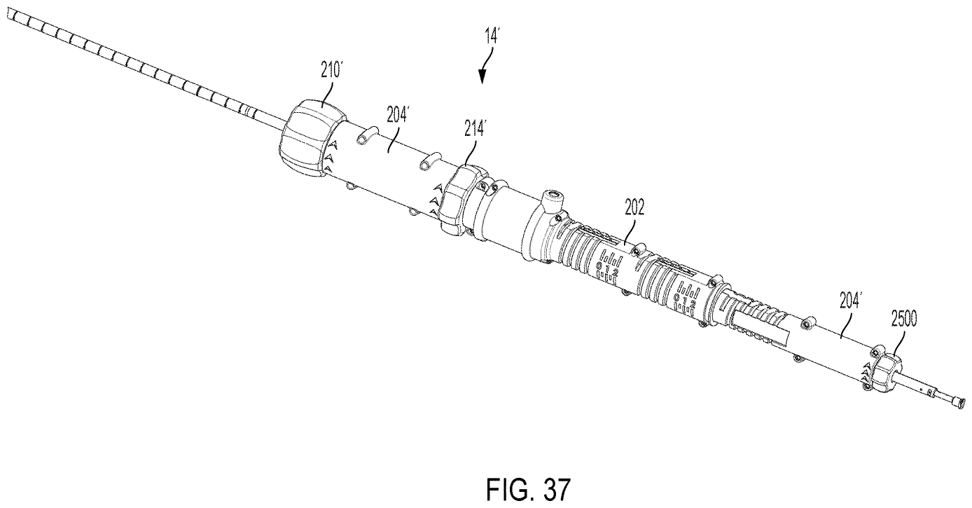

FIG. 37-45 illustrate an embodiment of a handle 14′ including control knobs 210′, 214′, 2500 that each have an outer grip surface that is exposed for gripping around an entire outer circumference of the respective control knob 210′, 214′, 2500. This is in contrast with the control knobs 210, 214 shown in FIG. 35 for example, in which a portion of the outer grip surface of the control knobs 210, 214 is covered by a bridge portion of the handle 14 that extends over the control knobs 210, 214.

The handle 14′ shown in FIG. 37 does not include bridges over the respective control knobs 210′, 214′, 2500, and accordingly each control knob 210′, 214′, 2500 has an outer grip surface that is exposed for gripping around an entire outer circumference of the respective control knob 210′, 214′, 2500. Such a feature may advantageously allow for greater grip force to be applied to the respective control knobs 210′, 214′, 2500, to better allow a user to rotate the knobs and move a respective portion of the delivery system to which the respective control knobs 210′, 214′, 2500 is coupled. The lack of presence of bridge portions of the handle 14 over the control knobs 210′, 214′, 2500 improves the ergonomics of the handle 14′ and allows for greater grip and forces to be applied by the user to the control knobs 210′, 214′, 2500.

The handle 14′ may include a rail housing 202 that is configured similarly as the rail housing 202 shown in FIG. 35 and coupled to the delivery housing 204′ in a similar manner as the rail housing 202 couples to the delivery housing 204. The pull wire knobs 206, 208 are not shown in FIGS. 37-45 for clarity, and the depth knob 212 is not shown for clarity as well, although the pull wire knobs 206, 208 and depth knob 212 would operate with the handle 14′ in the same manner as with the handle 14.

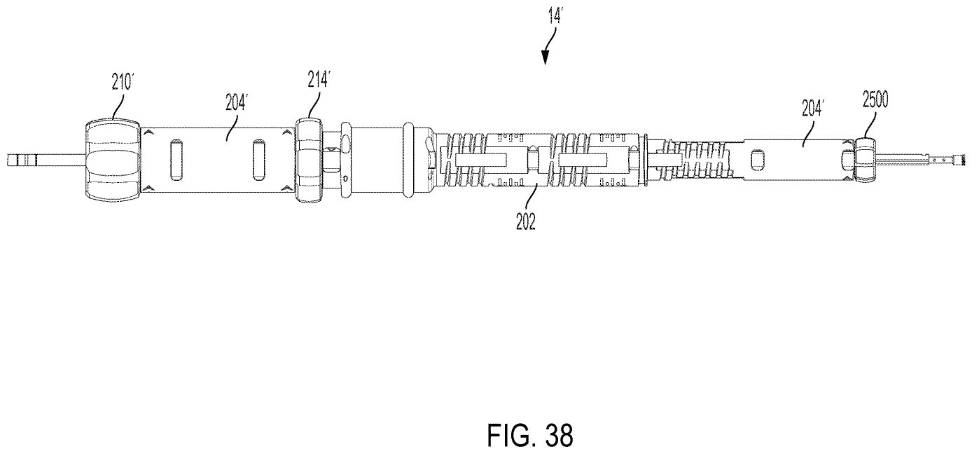

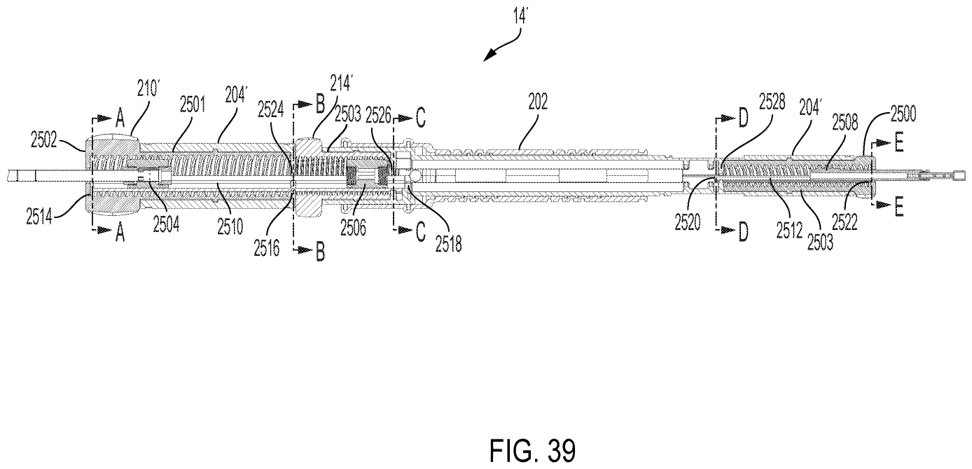

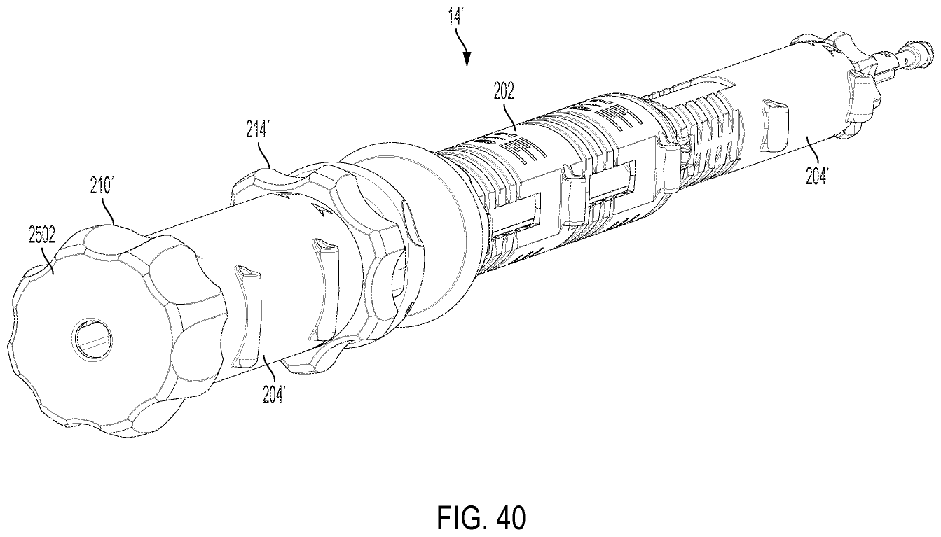

FIG. 37 illustrates a side perspective view of the handle 14′, and FIG. 38 illustrates a bottom view of the handle 14′. FIG. 39 illustrates a center cross sectional view of the handle 14′ from the bottom view of FIG. 38. The interior construction of the handle 14′ is shown in FIG. 39. The distalmost control knob 210′ is positioned at a distal end of the handle 14′ and is configured to control operation of the outer sheath assembly. A distal surface 2502 (visible in FIG. 40) forms the distal face of the handle 14′ and connects opposite sides of the grip surface of the control knob 210′. A middle control knob 214′ is positioned on the delivery housing 204′ and is configured to control operation of the mid shaft assembly. A proximal control knob 2500 is positioned at a proximal end of the handle 14′ and is configured to control operation of the nose cone assembly. The distalmost control knob 210′ and middle control knob 214′ may each be configured to be rotated to move their respective assemblies to release a portion of an implant from an implant retention area 16.

The delivery housing 204′ may include interior cavities that house respective sliders 2504, 2506, 2508 that are each coupled to the outer sheath assembly, the mid shaft assembly, and the nose cone assembly respectively. The sliders may be configured to slide along respective cavities that house the sliders 2504, 2506, 2508 to allow the sliders 2504, 2506, 2508 to translate the respective assemblies.

The control knobs 210′, 214′, 2500 may couple to bodies 2501, 2503, 2505 having threading that engages the threading of the sliders 2504, 2506, 2508, to allow the rotational motion of the control knob 210′, 214′, 2500 to produce axial or linear sliding of the respective slider 2504, 2506, 2508. One or more beams 2510, 2512 may be provided that may serve to prevent rotation of the sliders 2504, 2506, 2508 when the respective control knob 210′, 214′, 2500 rotates. Each beam 2510, 2512 may include a channel having a shape that is keyed to a shape of the respective slider 2504, 2506, 2508 to prevent rotation of the slider 2504, 2506, 2508 upon rotation of the control knob 210′, 214′, 2500. Only a portion of the threading of the bodies (e.g., half) may engage a slider to cause movement of the slider, as a portion of the threading of the bodies is covered by a respective beam. The respective slider 2504, 2506, 2508 is positioned within the channel of the beam and slides along the channel to cause movement of the assembly that the slider 2504, 2506, 2508 is coupled to.

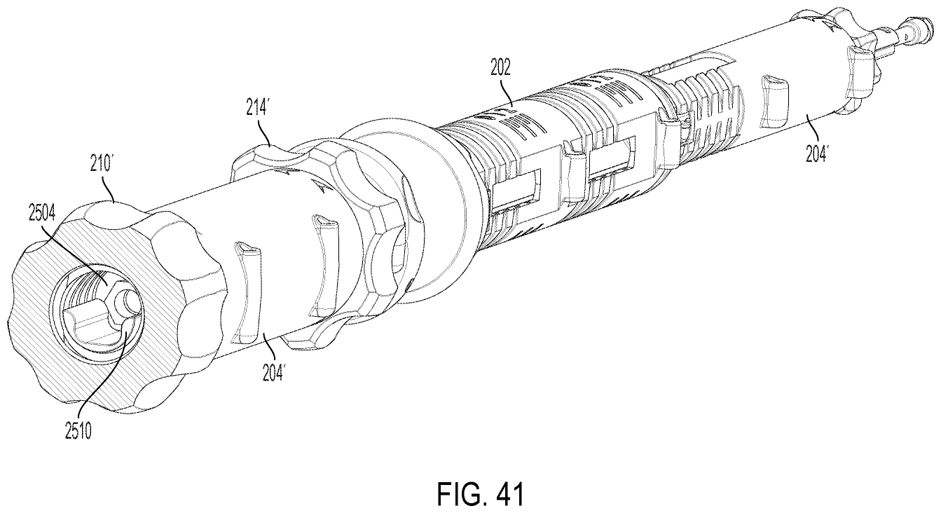

The beams 2510, 2512 may include walls that are positioned on both sides of an indentation to form the respective channel of the beams 2510, 2512. As such, the beams 2510, 2512 may have a “u” shape, as shown in FIGS. 41-45. Such a shape provides enhanced structural support for the handle 14′ and resists torque applied to the beams 2510, 2512 upon rotation of the control knobs 210′, 214′, 2500.

The beams 2510, 2512 may each extend within the cavities that house the sliders 2504 2506, 2508, and may be suspended within the cavities such that the threading of the control knobs 210′, 214′, 2500 does not engage or rotate the beams 2510, 2512. The beams 2510, 2512 may be supported by the delivery housing 204′ at supports 2514, 2516, 2518, 2520, 2522. The delivery housing 204′ may also include walls 2524, 2526, 2528 that separate the cavities of the delivery housing 204′ and serve to retain the beams 2510, 2512 such that the beams 2510, 2512 do not rotate with the respective cavities of the delivery housing 204′.

FIG. 40 illustrates a front perspective view of the handle 14′ with the elongate shaft not visible. The distal surface 2502 includes a central opening that allows the elongate shaft to pass through.

FIG. 41 illustrates a cross sectional view along line A-A in FIG. 39. The “u” shape of the beam 2510 is visible, as well as the keyed shape of the slider 2504.

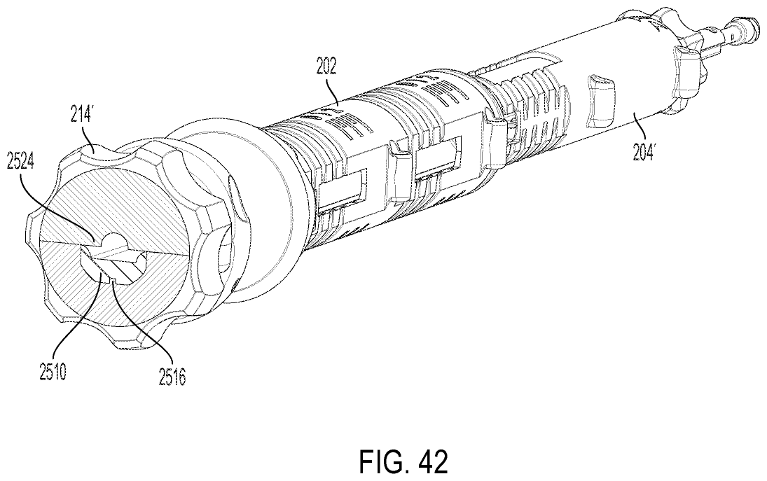

FIG. 42 illustrates a cross sectional view along line B-B in FIG. 39. The support 2516 shown as a protrusion entering a portion of the beam 2510. A support wall 2524 is adjacent to the beam 2510 to prevent rotation of the beam 2510 within the handle 14′.

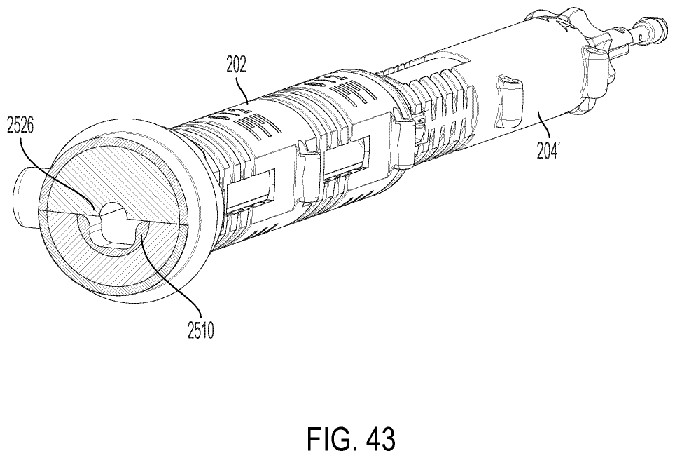

FIG. 43 illustrates a cross sectional view along line C-C in FIG. 39. A support wall 2526 is adjacent to the beam 2510 to prevent rotation of the beam 2510 within the handle 14′.

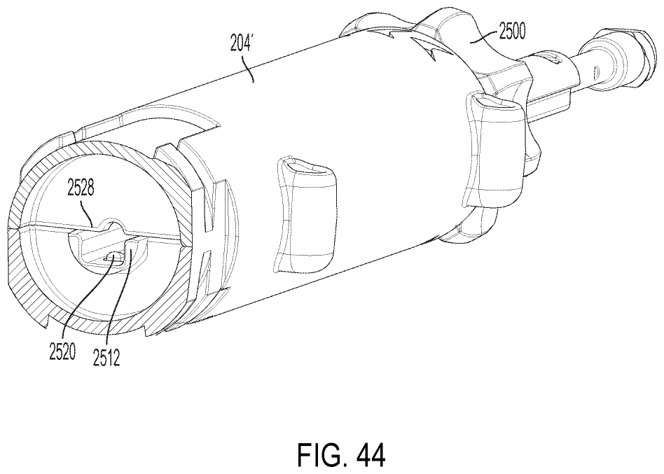

FIG. 44 illustrates a cross sectional view along line D-D in FIG. 39. The support 2520 is shown as a protrusion entering a portion of the beam 2512. A support wall 2528 is adjacent to the beam 2512 to prevent rotation of the beam 2512 within the handle 14′.

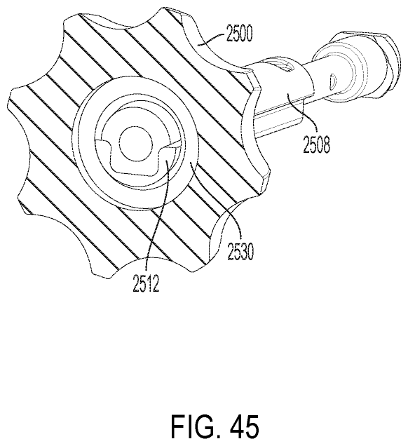

FIG. 45 illustrates a cross sectional view along line E-E in FIG. 39. The control knob 2500 is shown to include an unthreaded portion 2530 that serves as a stop to prevent the slider 2508 from being passed out of the proximal end of the handle 14′.

The handle 14′ may be configured to control a length of a path of travel of the respective sliders 2504, 2506, 2508 in a variety of manners. For example, a threading of the control knobs may be discontinued at a certain point to prevent travel of the respective sliders 2504, 2506, 2508. A size of the cavity that the slider 2504, 2506, 2508 travels in may be reduced as desired. In certain embodiments, stops in the form of protrusions may be placed along the beams 2510, 2512 or otherwise in the path of travel to control a length of a path of travel of the respective sliders 2504, 2506, 2508. In FIG. 39, the control knob 210′ serves as a distal stop to prevent distal axial movement of the slider 2504. The handle 14′ may be utilized with any embodiment of delivery system disclosed herein. The handle 14′ disclosed herein may be utilized solely, or may be utilized with any of the other apparatuses, systems, or methods disclosed herein.

Embodiments of the delivery systems disclosed herein may include markers that are configured to enhance an echogenicity of the elongate shaft of the delivery systems, to define a location of a portion of the elongate shaft when viewed under ultrasound imaging. Such markers may be positioned in a variety of locations of the elongate shaft, and may be positioned on a portion of the elongate shaft that is beneficially identified under ultrasound imaging to improve the delivery of the expandable implant. The ultrasound imaging may include echocardiography among other forms of ultrasound imaging.

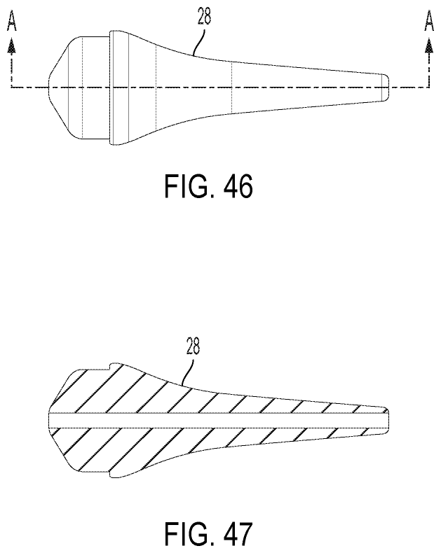

FIG. 46 illustrates a side view of a nose cone 28 of the delivery system. The nose cone 28 has a smooth tapered outer surface, which does not readily appear in ultrasound imaging. Further, FIG. 47 shows a cross sectional view of the nose cone 28 shown in FIG. 46. The nose cone 28 is shown to include a homogenous construction out of a single type of material, which is preferably soft and pliable such as a flexible polymer.

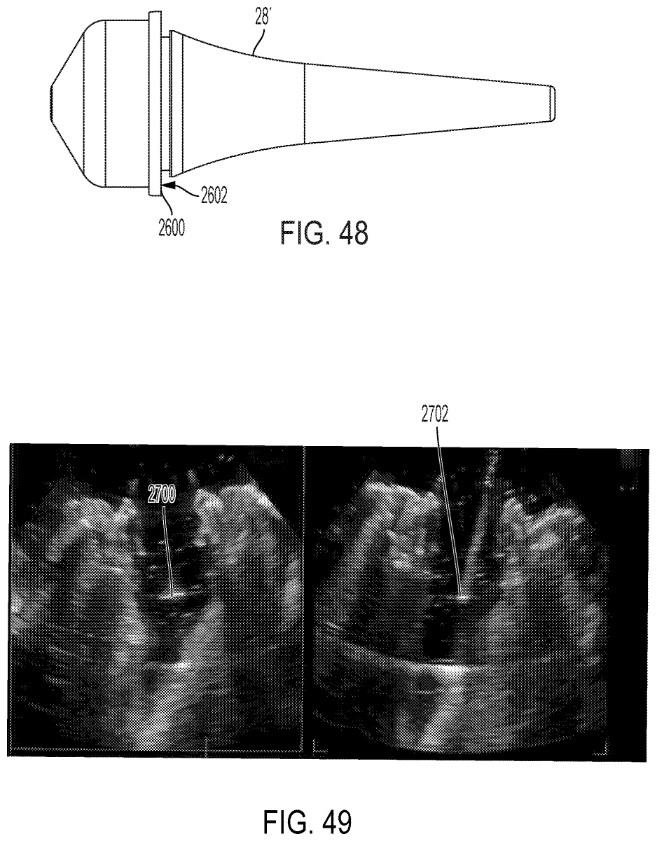

FIG. 48 illustrates a side view of a portion of the nose cone shown in FIG. 46 as nose cone 28′, and including a marker that is configured to enhance an echogenicity of the elongate shaft (particularly the nose cone 28′), to define a location of the portion (nose cone 28′) of the elongate shaft when viewed under ultrasound imaging. The marker as shown in FIG. 48 has the form of a contoured portion of the nose cone 28′ that forms an edge 2600 of the nose cone 28′. The edge 2600 extends around the circumference of the nose cone 28′ and forms a planar surface 2602 that extends around the circumference of the nose cone 28′. The planar surface 2602 faces distally. The edge 2600 forms an abrupt transition of acoustic impedance for the nose cone 28′, which enhances the echogenicity of the nose cone 28′. Further, the edge 2600 shape extending around the circumference of the nose cone 28′ enhances the acoustic reflectance for a variety of directions of incident ultrasound waves, thus also enhancing the echogenicity of the nose cone 28′.

FIG. 49 illustrates two echocardiogram images of the nose cone 28′ including the edge 2600 shown in FIG. 48. The edge 2600 is more brightly shown in the two echocardiogram images, as indicated by locations 2700 and 2702, which may allow the user to more easily locate the position of the nose cone 28′ in the images.

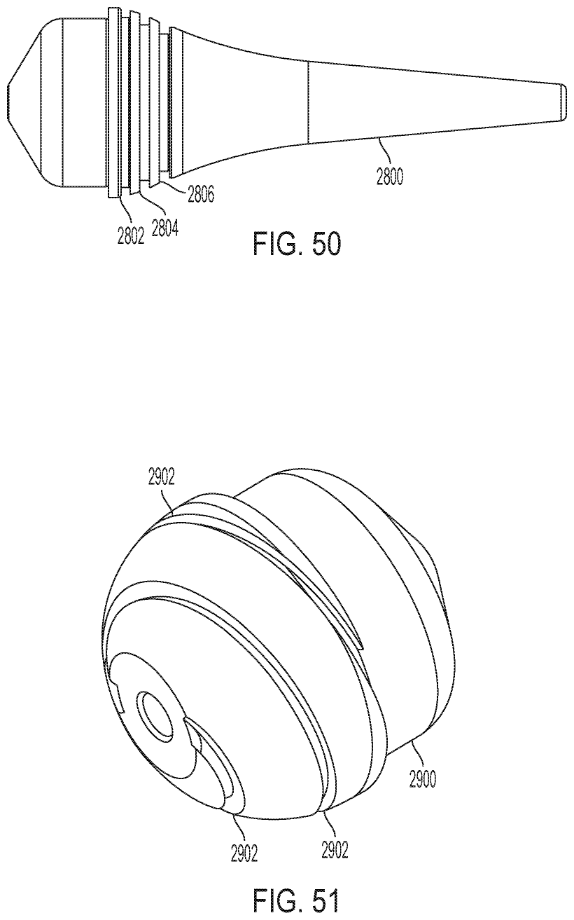

FIG. 50 illustrates an embodiment of a nose cone 2800 including a marker including a plurality of edges 2802, 2804, 2806 that enhance an echogenicity of the elongate shaft (particularly the nose cone 2800), to define a location of the portion (nose cone 2800) of the elongate shaft when viewed under ultrasound imaging. The plurality of edges 2802, 2804, 2806 may operate similarly as the edge 2600 shown in FIG. 48, and may each extend around the circumference of the nose cone 2800 and form respective distal facing planar surfaces that extend around the circumference of the nose cone 2800. A plurality of ledges may be formed by the plurality of edges 2802, 2804, 2806.

FIG. 51 illustrates an embodiment of a nose cone 2900 including a marker including an edge 2902 that enhance an echogenicity of the elongate shaft (particularly the nose cone 2900), to define a location of the portion (nose cone 2900) of the elongate shaft when viewed under ultrasound imaging. The edge 2902 may operate similarly as the edge 2600 shown in FIG. 48, and may each extend around the circumference of the nose cone 2900 and form a distal facing planar surface that extends around the circumference of the nose cone 2900. The edge 2902 may spiral around the nose cone 2900 to form a pattern in the shape of a spiral around the nose cone 2900.

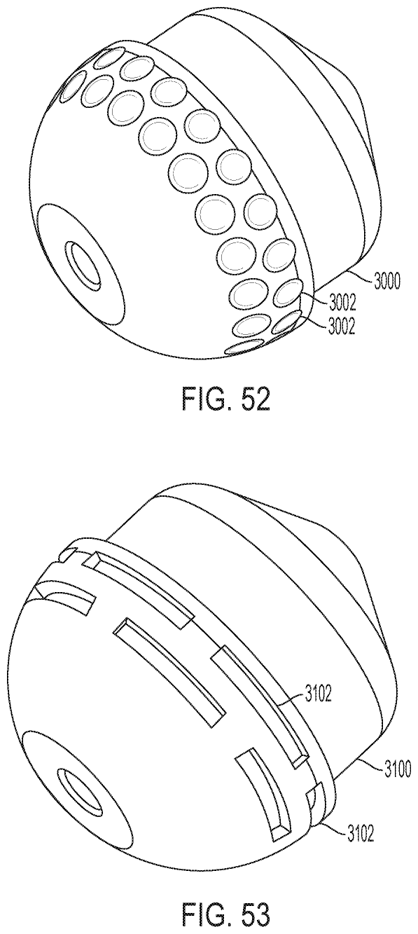

FIG. 52 illustrates an embodiment of a nose cone 3000 including a marker including a plurality of edges 3002 that enhance an echogenicity of the elongate shaft (particularly the nose cone 3000), to define a location of the portion (nose cone 3000) of the elongate shaft when viewed under ultrasound imaging. The edges 3002 may operate similarly as the edge 2600 shown in FIG. 48, and may form a pattern facing distally on the nose cone 3000. The pattern may include a plurality of indentations in the form of dimples on the nose cone 3000.

FIG. 53 illustrates an embodiment of a nose cone 3100 including a marker including a plurality of edges 3102 that enhance an echogenicity of the elongate shaft (particularly the nose cone 3100), to define a location of the portion (nose cone 3100) of the elongate shaft when viewed under ultrasound imaging. The edges 3102 may operate similarly as the edge 2600 shown in FIG. 48, and may form a pattern facing distally on the nose cone 3100. The pattern may include a plurality of indentations in the form of slots on the nose cone 3100.

The surfaces shown in FIG. 48, and FIGS. 50-53 may comprise the outer surfaces of the respective nose cones, or in other embodiments the markers may be encapsulated in material. The encapsulating material may comprise a material having different acoustic impedance than the markers, which may greater enhance the echogenicity of the markers. The encapsulating material may allow the nose cones to retain a smooth tapered surface.

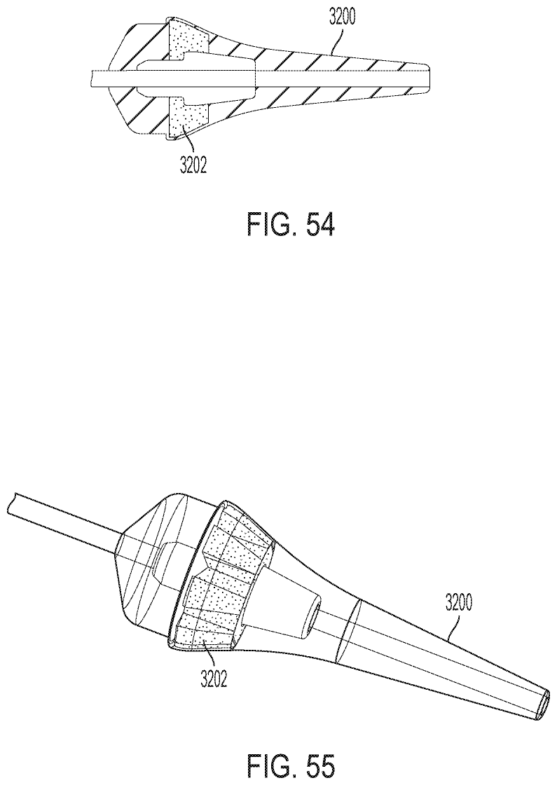

FIG. 54 illustrates a side cross sectional view of a nose cone 3200 including two materials having different acoustic impedances than each other. One material forms the marker 3202 shown in FIG. 54 and the other material forms adjacent encapsulating material having a different acoustic impedance than the material of the marker 3202 and that forms the outer surface of the nose cone 3200. The marker 3202 may be encapsulated such that the nose cone 3200 retains a smooth tapered surface.

FIG. 55 illustrates a perspective view of the nose cone 3200 including the marker 3202 The marker may include a plurality of edges, and may include multiple planar surfaces oriented along axial planes of the nose cone 3200 and planar surfaces oriented along the radial planes. The edges may form an abrupt transition of acoustic impedance for the nose cone 3200, which enhances the echogenicity of the nose cone 3200. Further, the edges and orientation of planar surfaces enhance the acoustic reflectance for a variety of directions of incident ultrasound waves, thus also enhancing the echogenicity of the nose cone 3200. The marker 3202 includes a plurality of fins extending radially outward.

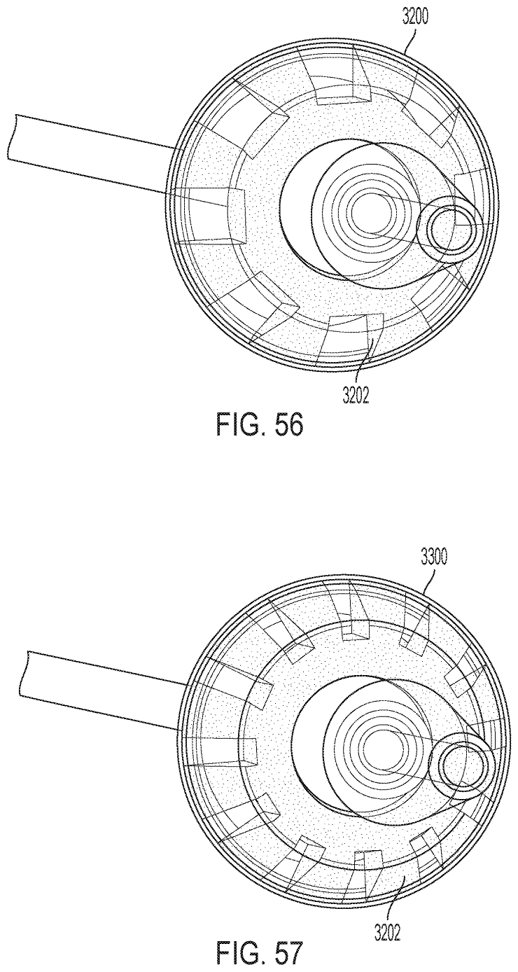

FIG. 56 illustrates a perspective view of the nose cone 3200 including the marker 3202 at a greater angle than shown in FIG. 55. The orientation of the plurality of fins is visible.

FIG. 57 illustrates a perspective view of a nose cone 3300 including a marker 3302 configured similarly as the marker 3202, but with a greater number of fins than shown in FIG. 56.

The markers disclosed herein may be made of a material having a relatively high acoustic impedance, which may comprise metals or the like. The acoustic impedance may be greater than the impedance of an adjacent material to allow for enhanced echogenicity resulting from use of the marker.

The markers disclosed herein may be utilized in a variety of locations of the elongate shaft and not only on the nose cone comprising the tip of the elongate shaft. For example, the capsule, the outer retention ring of the mid shaft, and other locations on the outer sheath assembly, the mid shaft assembly, and the inner shaft assembly may include the markers as desired.

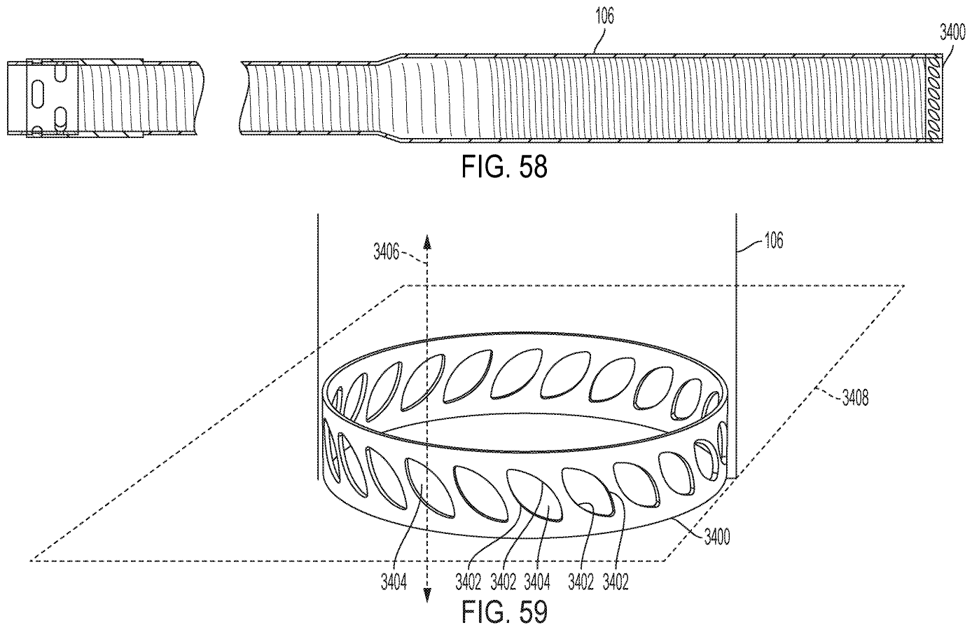

FIG. 58 illustrates a side cross sectional view of a capsule 106 including a marker 3400 at a distal end of the capsule 106. The marker 3400 may include a plurality of edges that enhance an echogenicity of the elongate shaft (particularly the distal end of the capsule 106), to define a location of the portion (the distal end of the capsule 106) of the elongate shaft when viewed under ultrasound imaging.

FIG. 59 illustrates a close up perspective view of the marker 3400. The marker may include the plurality of edges 3402 that bound apertures 3404 of the marker 3400. The plurality of apertures 3404 may be spaced circumferentially about the marker 3400. The apertures 3404 may allow for a transition of acoustic impedance between the body of the marker 3400 and the apertures 3404, which may be filled with a material having a different acoustic impedance than the body of the marker 3400. The apertures 3404 may each have an oblong shape in which adjacent apertures 3404 overlap each other, such that a vertical scan slice taken of the marker along a vertical dimension, as represented by line 3406, will pass through the apertures 3404 and allow for the transition in acoustic impedance caused by the apertures 3404. Further, the angled profile of the apertures 3404 may enhance the acoustic reflectance for a variety of directions of incident ultrasound waves. The marker 3400 in the form of a ring extends about a longitudinal axis of the elongate shaft, and the plurality of apertures are positioned circumferentially about the longitudinal axis.

The position of the apertures 3404 vertically upon the body of the marker 3400 will also enhance the possibility of an acoustic slice passing through the apertures 3404 when extending in a transverse dimension, as represented by plane 3408 in FIG. 59.

The marker 3400 may comprise a band of material having a different acoustic impedance than adjacent material, and may be positioned as desired. The marker 3400 for example, may be positioned on the outer retention ring of the mid shaft, and on the nose cone or tip. Other locations on the outer sheath assembly, the mid shaft assembly, and the inner shaft assembly may include the markers as desired.

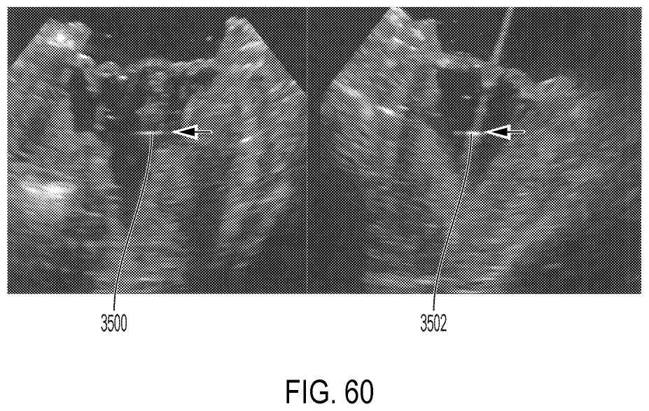

FIG. 60 illustrates two echocardiogram images of the capsule including the marker 3400. The marker 3400 is more brightly shown in the two echocardiogram images, as indicated by locations 3500 and 3502, which may allow the user to more easily locate the position of the capsule in the images. The markers may be coupled to a tip of the elongate shaft, or in any other location, and may appear more brightly viewed with ultrasound imaging such as echocardiography than remaining portions of the tip or remaining portions of the elongate shaft.

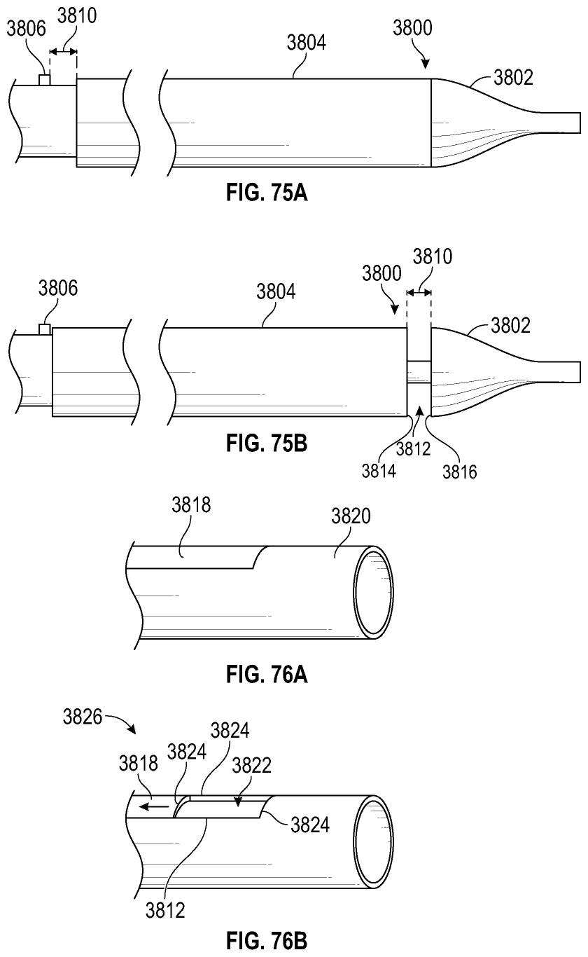

The markers may be configured to be activated in embodiments herein. The markers may be configured to have a greater echogenicity upon being activated. An activation mechanism, or another system, may be utilized to activate the markers as disclosed herein. FIG. 75A, for example, illustrates an embodiment of a marker 3800 that is configured to be activated. A distal end of a delivery system is shown in FIG. 75A, including a nose cone 3802 and a capsule 3804. The outer surface of the nose cone 3802 and the outer surface of the capsule 3804 may comprise a smooth surface forming a smooth outer profile in the configuration shown in FIG. 75A. In such a configuration, for example, a delivery system may be passed through the patient's body, with the smooth outer profile reducing the possibility of damaging a surface of the patient's body.

The capsule 3804 may be configured to be retracted to activate the marker 3800 and allow the marker 3800 to enhance the echogenicity of the elongate shaft of the delivery system. An activation mechanism accordingly may comprise the capsule 3804 and an indicator 3806 that may be positioned on a proximal portion of the elongate shaft. The indicator 3806 may be configured as a stop as shown in FIG. 75A, or may be configured as a tactile indicator, audible indicator, or other form of indicator in embodiments.

Upon the user desiring to activate the marker 3800, a first portion of the elongate shaft may be moved relative to a second portion of the elongate shaft to activate the marker 3800. For example, the capsule 3804 may be retracted a length 3810 (marked in FIGS. 75A and 75B) to form a gap 3812 (marked in FIG. 75B) between two portions of the delivery system (e.g., the capsule 3804 and the nose cone 3802). FIG. 75B, for example, illustrates the capsule 3804 retracted by the length 3810. The capsule 3804 moves axially relative to the nose cone 3802.

Referring to FIG. 75B, the gap 3812 formed by the movement of the capsule 3804 may expose the respective edges 3814, 3816 of the capsule 3804 and the nose cone 3802 thus activating the marker 3800. In such a configuration, a user may be able to identify the marker 3800 with ultrasound imaging. The marker 3800 includes a contoured portion forming edges 3814, 3816 of the elongate shaft. A planar surface formed by edge 3814 may face distally, and a planar surface formed by edge 3816 may face proximally.

In embodiments, the indicator 3806 may indicate to the user to only retract the capsule 3804 a relatively short distance, to maintain a proximity between the edges 3814, 3816. The proximity of the edges 3814, 3816 may enhance the echogenicity of the marker 3800. Upon the user desiring to further retract the capsule 3804 (e.g. to deploy the implant), the indicator 3806 may be overcome, for example, by overcoming a stop or otherwise continuing movement past the indicator 3806. For example, the indicator 3806 may be moved (such as being pressed or slid) such that the capsule 3804 may continue to be retracted.

In such a configuration, the portion of the delivery system including the marker 3800 may have a smooth outer profile, with the edges 3814, 3816 exposed only at a desired time by the user operating the activation mechanism. Thus, the possibility of damage to the patient's body caused by an uneven outer profile may be reduced until a desired time of activation of the marker 3800.

Other configurations of activatable markers may be utilized in embodiments. FIG. 76A, for example, illustrates an embodiment in which a first portion of the elongate shaft (e.g., strip of material 3818) forms an outer surface of the delivery system (e.g., an outer surface of a capsule 3820). The portion as shown may comprise a capsule of the elongate shaft or another portion as desired. The strip of material 3818 may be moved relative to a second portion of the elongate shaft (e.g., an adjacent portion of the elongate shaft) to form a gap 3822 between the portions. A user may operate an activation mechanism as disclosed herein for example. The gap 3822 may expose edges 3824 of the marker 3826 (as marked in FIG. 76B). The material 3818 may be slid axially.

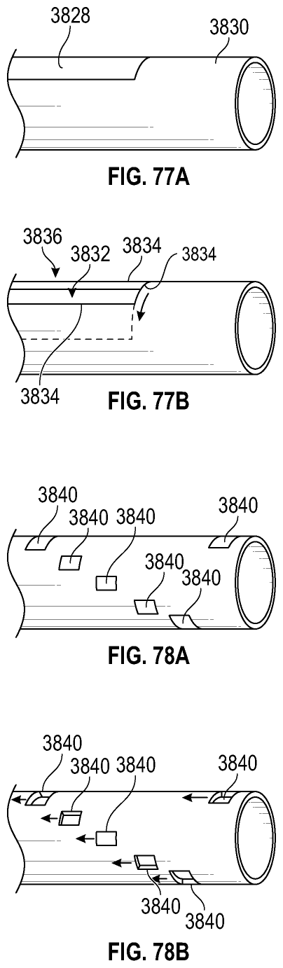

FIG. 77A, illustrates an embodiment in which a first portion of the elongate shaft (e.g., strip of material 3828) forms an outer surface of the delivery system (e.g., an outer surface of a capsule 3830). The strip of material 3828 may be moved to form a gap 3832 and expose edges 3834 of the marker 3836 (as marked in FIG. 77B). The material 3828 may be rotated relative to the second portion of the elongate shaft (e.g., an adjacent portion of the elongate shaft) to activate the marker 3836. A user may operate an activation mechanism as disclosed herein for example to rotate the strip of material 3828.

In embodiments, a plurality of markers may be positioned on the delivery system. The markers may be spaced from each other at a distance such that a user viewing the markers on ultrasound imaging is able to identify a relative location on the delivery system. FIG. 78A, for example, illustrates an embodiment in which the plurality of markers 3840 have graduated spacing on the delivery system. The markers 3840 may form a pattern in a spiral. As such, upon activation of the markers 3840 (as shown in FIG. 78B), a plurality of locations on the delivery system are indicated. A user may operate an activation mechanism as disclosed herein for example. Strips of material forming an outer surface of the delivery system may each be moved to form gaps and expose the edges of the markers 3840. A user may be able to match the location of the marker 3840 with a respective location on the delivery system. Further, a user may be able to determine a relative scaling on the imaging system based on distance imaged between the markers 3840. For example, if the user is aware that each marker is 3 millimeters apart from each other, then that scaling may be utilized to determine distance viewed on the imaging system.

The use of markers disclosed herein may more easily allow a user to locate a portion of the elongate shaft under ultrasound imaging, which may include echocardiography. The user may more easily be able to determine a deployment location for the expandable implant, which may include a depth of the expandable implant and a relation of the expandable implant relative to structures of the patient's heart, including papillary muscles and any valve flaps, including mitral valve flaps. The user beneficially may visualize the location of portions of the elongate shaft without relying solely on fluoroscopy for visualization. A marker as disclosed herein may be utilized on any part of a catheter, intravenous, or gastrointestinal system or implant or any other device for insertion into a human body where a specific location needs to be identified with ultrasound imaging or with echocardiography. The markers disclosed herein may be utilized solely, or may be utilized with any of the other apparatuses, systems, or methods disclosed herein.

Methods of using the delivery system 10 in connection with a replacement mitral valve will now be described. In particular, the delivery system 10 can be used in a method for percutaneous delivery of a replacement mitral valve to treat patients with moderate to severe mitral regurgitation. The below methods are merely examples of the how the delivery system may be used. It will be understood that the delivery systems described herein can be used as part of other methods as well. The embodiments shown in FIG. 16-60 may be incorporated and utilized as desired.

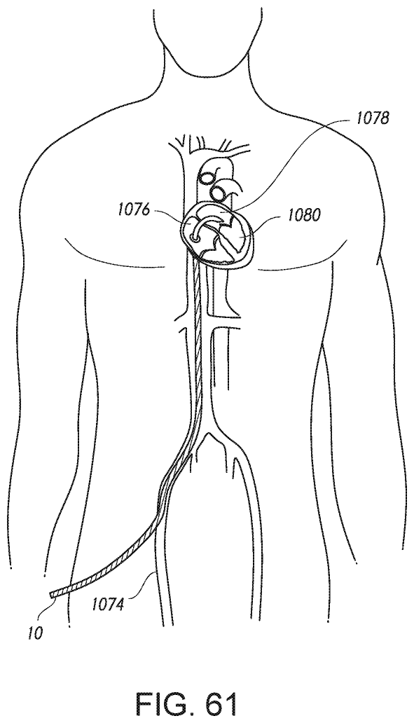

As shown in FIG. 61, in one embodiment the delivery system 10 can be placed in the ipsilateral femoral vein 1074 and advanced toward the right atrium 1076. A transseptal puncture using known techniques can then be performed to obtain access to the left atrium 1078. The delivery system 10 can then be advanced in to the left atrium 1078 and then to the left ventricle 1080. FIG. 61 shows the delivery system 10 extending from the ipsilateral femoral vein 1074 to the left atrium 1078. In embodiments of the disclosure, a guide wire is not necessary to position the delivery system 10 in the proper position, although in other embodiments, one or more guide wires may be used.

Accordingly, it can be advantageous for a user to be able to steer the delivery system 10 through the complex areas of the heart in order to position a replacement mitral valve in line with the native mitral valve. This task can be performed with or without the use of a guide wire with the above disclosed system. The distal end of the delivery system can be advanced into the left atrium 1078. A user can then manipulate the rail assembly 20 to target the distal end of the delivery system 10 to the appropriate area. A user can then continue to pass the bent delivery system 10 through the transseptal puncture and into the left atrium 1078. A user can then further manipulate the delivery system 10 to create an even greater bend in the rail assembly 20. Further, a user can torque the entire delivery system 10 to further manipulate and control the position of the delivery system 10. In the fully bent configuration, a user can then place the replacement mitral valve in the proper location. This can advantageously allow delivery of a replacement valve to an in-situ implantation site, such as a native mitral valve, via a wider variety of approaches, such as a transseptal approach.

The rail assembly 20 can be particularly advantageous for entering into the native mitral valve. As discussed above, the rail assembly 20 can form two bends, both of which can be located in the left atrium 1078. The bends in the rail assembly 20 can position the implant 70, located in the implant retention area 16, so that it is coaxial with the native mitral valve. Once the implant 70 is coaxial, the outer sheath assembly 22, mid shaft assembly 21, inner assembly 18, and nose cone assembly 31 can together be advanced (e.g., using the depth knob 212 of the handle 14) distally relative to the rail assembly 20. These assemblies advance straight off of the rail assembly 20, thus advancing them coaxial with the native mitral valve until the implant 70 is to be released while maintain the implant 70 in the compressed configuration, as discussed below. Thus, the rail assembly 20 provides the ability for a user to lock the angular position in place, so that the user then has to just longitudinally advance the other assemblies over the rail assembly 20 while not needed to make any angular changes, greatly simplifying the procedure. The rail assembly 20 acts as an independent steering assembly, where all the assembly does is provide steerability and no further prosthesis release functionality. Further, the construction of the rail assembly 20 as described above is sufficiently rigid so that when the rail assembly is actuated to its bent shape, movement of the other components, e.g., the outer sheath assembly 22, mid shaft assembly 21, inner assembly 18, and/or nose cone assembly 31, the rail assembly 20 maintains its shape. Thus, the rail assembly 20 can remain in the desired bent position during the sliding of the other assemblies relative to the rail assembly 20, and the rail assembly 20 can help direct the other assemblies to the final position. The proximal/distal translation of the other assemblies over the rail assembly 20 allows for ventricular-atrial motion. In addition, once the distal anchors 80 of the implant 70 have been released in the left ventricle 1080, but prior to full release, the other assemblies can be proximally retracted over the rail assembly 20 to capture any leaflets or chordae.

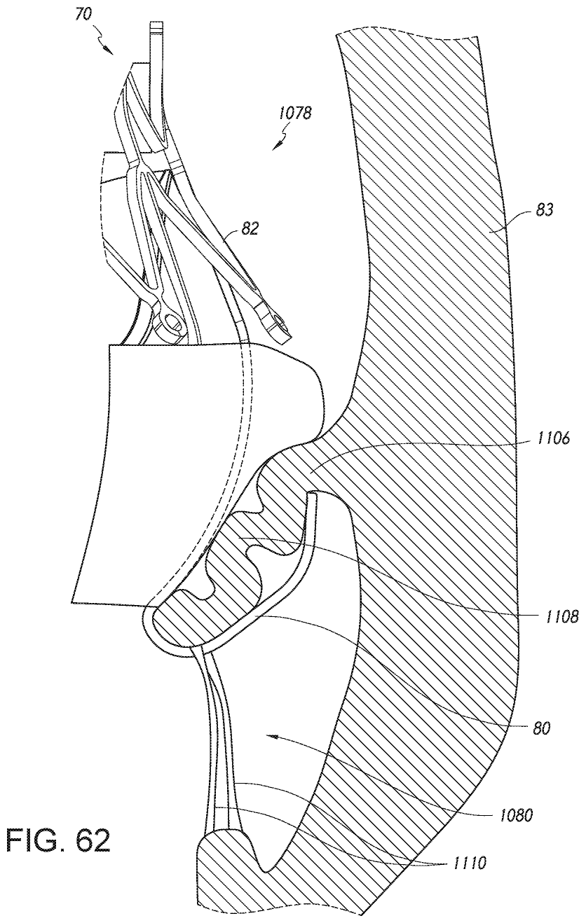

Reference is now made to FIG. 62 which illustrates a schematic representation of a portion of an embodiment of a replacement heart valve (implant 70) positioned within a native mitral valve of a heart 83. Further details regarding how the implant 70 may be positioned at the native mitral valve are described in U.S. Publication No. 2015/0328000A1, the entirety of which is hereby incorporated by reference, including but not limited to FIGS. 13A-15 and paragraphs [0036]-[0045]. A portion of the native mitral valve is shown schematically and represents typical anatomy, including a left atrium 1078 positioned above an annulus 1106 and a left ventricle 1080 positioned below the annulus 1106. The left atrium 1078 and left ventricle 1080 communicate with one another through a mitral annulus 1106. Also shown schematically in FIG. 62 is a native mitral leaflet 1108 having chordae tendineae 1110 that connect a downstream end of the mitral leaflet 1108 to the papillary muscle of the left ventricle 1080. The portion of the implant 70 disposed upstream of the annulus 1106 (toward the left atrium 1078) can be referred to as being positioned supra-annularly. The portion generally within the annulus 1106 is referred to as positioned intra-annularly. The portion downstream of the annulus 1106 is referred to as being positioned sub-annularly (toward the left ventricle 1080).

As shown in FIG. 62, the replacement heart valve (e.g., implant 70) can be positioned so that the mitral annulus 1106 is located between the distal anchors 80 and the proximal anchors 82. In some situations, the implant 70 can be positioned such that ends or tips of the distal anchors 80 contact the annulus 1106 as shown, for example, in FIG. 62. In some situations, the implant 70 can be positioned such that ends or tips of the distal anchors 80 do not contact the annulus 1106. In some situations, the implant 70 can be positioned such that the distal anchors 80 do not extend around the leaflet 1108.

As illustrated in FIG. 62, the implant 70 in the form of a prosthesis, specifically a replacement heart valve, can be positioned so that the ends or tips of the distal anchors 80 are on a ventricular side of the mitral annulus 1106 and the ends or tips of the proximal anchors 82 are on an atrial side of the mitral annulus 1106. The distal anchors 80 can be positioned such that the ends or tips of the distal anchors 80 are on a ventricular side of the native leaflets beyond a location where chordae tendineae 1110 connect to free ends of the native leaflets. The distal anchors 80 may extend between at least some of the chordae tendineae 1110 and, in some situations such as those shown in FIG. 62, can contact or engage a ventricular side of the annulus 1106. It is also contemplated that in some situations, the distal anchors 80 may not contact the annulus 1106, though the distal anchors 80 may still contact the native leaflet 1108. In some situations, the distal anchors 80 can contact tissue of the left ventricle 1080 beyond the annulus 1106 and/or a ventricular side of the leaflets.

During delivery, the distal anchors 80 (along with the frame) can be moved toward the ventricular side of the annulus 1106, such as by translating the other assemblies (e. g,. outer sheath assembly 22, mid shaft assembly 21, inner assembly 18, and nose cone assembly 31) proximally with respect to the rail assembly 20, with the distal anchors 80 extending between at least some of the chordae tendineae 1110 to provide tension on the chordae tendineae 1110. The degree of tension provided on the chordae tendineae 1110 can differ. For example, little to no tension may be present in the chordae tendineae 1110 where the leaflet 1108 is shorter than or similar in size to the distal anchors 80. A greater degree of tension may be present in the chordae tendineae 1110 where the leaflet 1108 is longer than the distal anchors 80 and, as such, takes on a compacted form and is pulled proximally. An even greater degree of tension may be present in the chordae tendineae 1110 where the leaflets 1108 are even longer relative to the distal anchors 80. The leaflet 1108 can be sufficiently long such that the distal anchors 80 do not contact the annulus 1106.

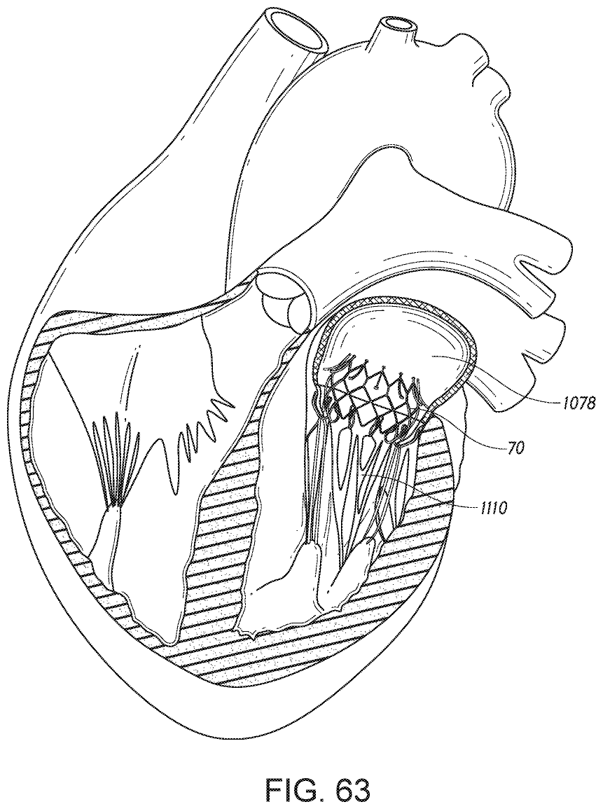

The proximal anchors 82, if present, can be positioned such that the ends or tips of the proximal anchors 82 are adjacent the atrial side of the annulus 1106 and/or tissue of the left atrium 1078 beyond the annulus 1106. In some situations, some or all of the proximal anchors 82 may only occasionally contact or engage atrial side of the annulus 1106 and/or tissue of the left atrium 1078 beyond the annulus 1106. For example, as illustrated in FIG. 62, the proximal anchors 82 may be spaced from the atrial side of the annulus 1106 and/or tissue of the left atrium 1078 beyond the annulus 1106. The proximal anchors 82 could provide axial stability for the implant 70. It is also contemplated that some or all of the proximal anchors 82 may contact the atrial side of the annulus 1106 and/or tissue of the left atrium 1078 beyond the annulus 1106. FIG. 63 illustrates the implant 70 implanted in the heart. Although the illustrated replacement heart valve includes both proximal and distal anchors, it will be appreciated that proximal and distal anchors are not required in all cases. For example, a replacement heart valve with only distal anchors may be capable of securely maintaining the replacement heart valve in the annulus. This is because the largest forces on the replacement heart valve are directed toward the left atrium during systole. As such, the distal anchors are most important for anchoring the replacement heart valve in the annulus and preventing migration.

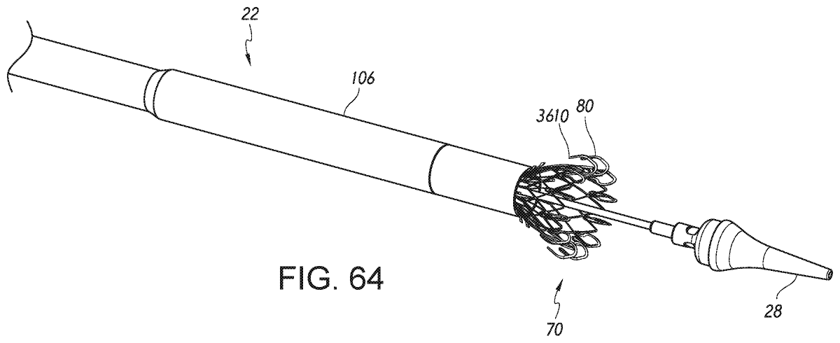

FIGS. 64-66 illustrate the release mechanism of the delivery system 10. During the initial insertion of the implant 70 and the delivery system 10 into the body, the implant 70 can be located within the system 10, similar to as shown in FIG. 2A. The distal end 303 of the implant 70, and specifically the distal anchors 80, are restrained within the capsule 106 of the outer sheath assembly 22, thus preventing expansion of the implant 70. Similar to what is shown in FIG. 2A, the distal anchors 80 can extend distally when positioned in the capsule. The proximal end 301 of the implant 70 is restrained within the capsule 106 and within a portion of the inner retention member 40 and thus is generally constrained between the capsule 106 and the inner retention member 40.

The system 10 can first be positioned to a particular location in a patient's body, such as at the native mitral valve, through the use of the steering mechanisms discussed herein or other techniques.

Once the implant 70 is loaded into the delivery system 10, a user can thread a guide wire into a patient to the desired location. The guide wire passes through the lumen of the nose cone assembly 31, and thus the delivery system 10 can be generally advanced through the patient's body following the guide wire. The delivery system 10 can be advanced by the user manually moving the handle 14 in an axial direction. In some embodiments, the delivery system 10 can be placed into a stand while operating the handle 14 controls.

Once generally in heart, the user can begin the steering operation of the rail assembly 2 using the distal pull wire knob 206 and/or the proximal pull wire knob 208. By turning either of the knobs, the user can provide flexing/bending of the rail assembly 20 (either on the distal end or the proximal end), thus bending the distal end of the delivery system 10 in one, two, or more locations into the desired configuration. As discussed above, the user can provide multiple bends in the rail assembly 20 to direct the delivery system 10 towards the mitral valve. In particular, the bends of the rail assembly 20 can direct a distal end of the delivery system 10, and thus the capsule 106, along the center axis passing through the native mitral valve. Thus, when the outer sheath assembly 22, mid shaft assembly 21, inner assembly 18, and nose cone assembly 31 are together advanced over the rail assembly 20 with the compressed implant 70, the capsule 106 proceed directly in line with the axis for proper release of the implant 70.

The user can also rotate and/or move the handle 14 itself in a stand for further fine tuning of the distal end of the delivery system 10. The user can continually turn the proximal and/or distal pull wire knobs 208/206, as well as moving the handle 14 itself, to orient the delivery system 10 for release of the implant 70 in the body. The user can also further move the other assemblies relative to the rail assembly 20, such as proximally or distally.

In a next step, the user can rotate the depth knob 212. As discussed, rotation of this knob 212 together advances the inner shaft assembly 18, mid shaft assembly 21, outer sheath assembly 22, and nose cone assembly 31 over/through the rail assembly 20 while the implant 70 remains in the compressed configuration within the implant retention area 16. Due to the rigidity of, for example, either the inner shaft assembly 18, the mid shaft assembly 21, and/or the outer sheath assembly 22, these assemblies proceed straight forward in the direction aligned by the rail assembly 20.

Once in the release position, the user can rotate the outer sheath knob 210, which individually translates the outer sheath assembly 22 (and thus the capsule 106) with respect to the other assemblies, in particular the inner assembly 18, in a proximal direction towards the handle 14 as shown in FIG. 64. By doing so, the distal end 303 of implant 70 is uncovered in the body, allowing for the beginning of expansion. At this point, the distal anchors 80 can flip proximally and the distal end 303 begins to expand radially outwardly. For example, if the system 10 has been delivered to a native mitral valve location through a transseptal approach, the nose cone is positioned in the left ventricle, preferably aligning the implant 70 such that it is generally perpendicular to the plane of the mitral annulus. The distal anchors 80 expand radially outwardly within the left ventricle. The distal anchors 80 can be located above the papillary heads, but below the mitral annulus and mitral leaflets. In some embodiments, the distal anchors 80 may contact and/or extend between the chordae in the left ventricle, as well as contact the leaflets, as they expand radially. In some embodiments, the distal anchors 80 may not contact and/or extend between the chordae or contact the leaflets. Depending on the position of the implant 70, the distal ends of the distal anchors 80 may be at or below where the chordae connect to the free edge of the native leaflets.

As shown in the illustrated embodiment, the distal end 303 of the implant 70 is expanded outwardly. It should be noted that the proximal end 301 of the implant 70 can remain covered by the outer retention ring during this step such that the proximal end 301 remains in a radially compacted state. At this time, the system 10 may be withdrawn proximally so that the distal anchors 80 capture and engage the leaflets of the mitral valve, or may be moved proximally to reposition the implant 70. For example, the assemblies may be proximally moved relative to the rail assembly 20. Further, the system 10 may be torqued, which may cause the distal anchors 80 to put tension on the chordae through which at least some of the distal anchors may extend between. However, in some embodiments the distal anchors 80 may not put tension on the chordae. In some embodiments, the distal anchors 80 may capture the native leaflet and be between the chordae without any further movement of the system 10 after withdrawing the outer sheath assembly 22.

During this step, the system 10 may be moved proximally or distally to cause the distal or ventricular anchors 80 to properly capture the native mitral valve leaflets. This can be done by moving the outer sheath assembly 22, mid shaft assembly 21, inner assembly 18, and nose cone assembly 31 with respect to the rail assembly 20. In particular, the tips of the ventricular anchors 80 may be moved proximally to engage a ventricular side of the native annulus, so that the native leaflets are positioned between the anchors 80 and the body of the implant 70. When the implant 70 is in its final position, there may or may not be tension on the chordae, though the distal anchors 80 can be located between at least some of the chordae.

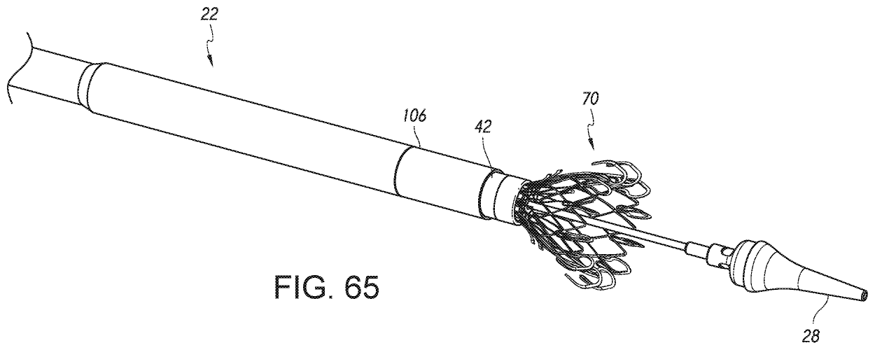

The proximal end 301 of the implant 70 will remain in the outer retention ring 42 after retraction of the capsule 106. As shown in FIG. 65, once the distal end 303 of the implant 70 is fully expanded (or as fully expanded as possible at this point), the outer retention ring 42 can be individually withdrawn proximally with respect to the other assemblies, in particular relative to the inner assembly 18, to expose the inner retention member 40, thus beginning the expansion of the proximal end 301 of the implant 70. For example, in a mitral valve replacement procedure, after the distal or ventricular anchors 80 are positioned between at least some of the chordae tendineae and/or engage the native mitral valve annulus, the proximal end 301 of the implant 70 may be expanded within the left atrium.

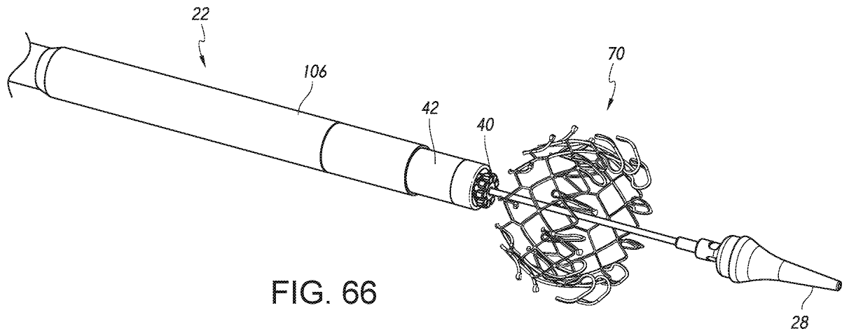

The outer retention ring 42 can be moved proximally such that the proximal end 310 of the implant 70 can radially expand to its fully expanded configuration as shown in FIG. 66. After expansion and release of the implant 70, the inner assembly 18, nose cone assembly 31, mid shaft assembly 21, and outer sheath assembly 22 can be simultaneously withdrawn proximally along or relative to the rail assembly 20 back to their original position. In some embodiments, they are not withdrawn relative to the rail assembly 20 and remain in the extended position. Further, the nose cone 28 can be withdrawn through the center of the expanded implant 70 and into the outer sheath assembly 22, such as by proximally translating the knob 216. The system 10 can then be removed from the patient.

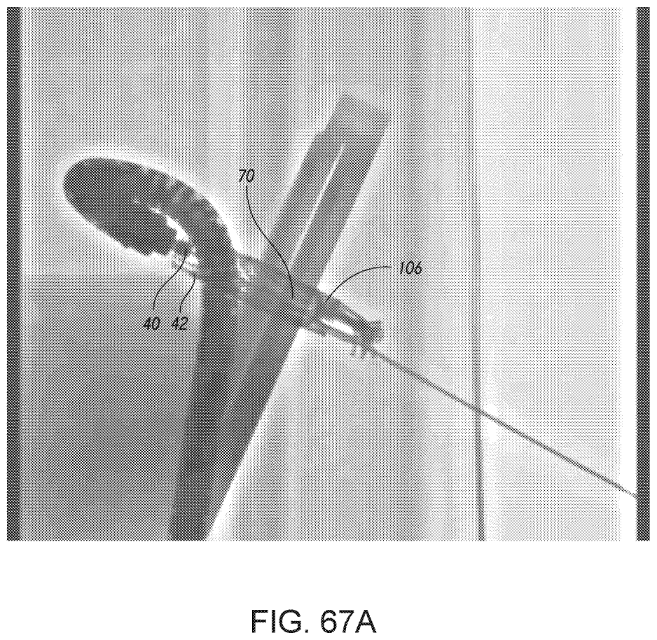

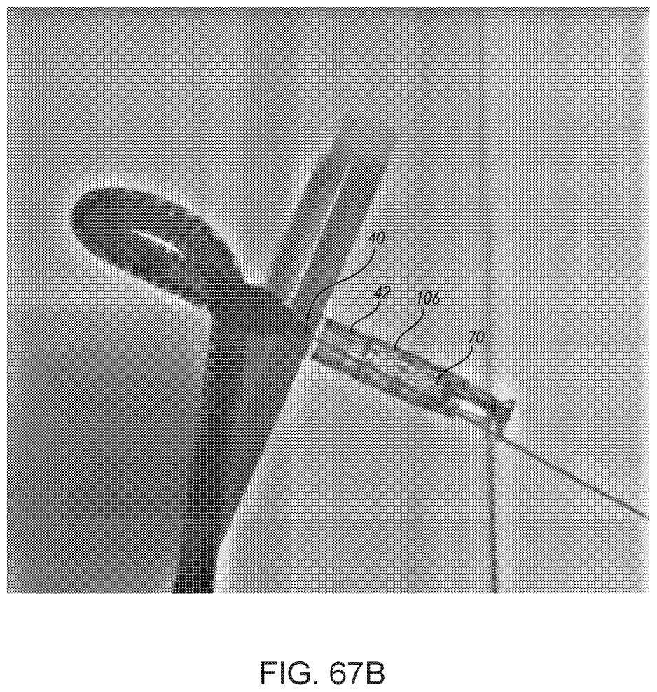

FIGS. 67A-B illustrate the advancement of the different assemblies over the rail assembly 20. FIG. 67A illustrates the assemblies in their proximalmost position over the rail assembly 20. FIG. 67B illustrates the assemblies in their distalmost position as compared to the rail assembly 20, such as shown in FIG. 2C. Thus, the assemblies snake along the rail assembly 20 and extend distally away.

In some embodiments, the implant 70 can be delivered under fluoroscopy so that a user can view certain reference points for proper positioning of the implant 70. Further, echocardiography can be used for proper positioning of the implant 70.

Following is a discussion of an alternative implantation method for delivering a replacement mitral valve to a mitral valve location. Elements of the below can be incorporated into the above discussion and vice versa. Prior to insertion of the delivery system 10, the access site into the patient can be dilated. Further, a dilator can be flushed with, for example, heparinized saline prior to use. The delivery system 10 can then be inserted over a guide wire. In some embodiments, any flush ports on the delivery system 10 can be pointed vertically. Further, if an introducer tube is used, integrated or otherwise, this can be stabilized. The delivery system 10 can be advanced through the septum until a distal end of the delivery system 10 is positioned across the septum into the left atrium 1078. Thus, the distal end of the delivery system 10 can be located in the left atrium 1078. In some embodiments, the delivery system 10 can be rotated, such as under fluoroscopy, into a desired position. The rail can be flex so that direct a distal end of the delivery system 10 towards the septum and mitral valve. The position of the delivery system 10, and the implant 70 inside, can be verified using echocardiography and fluoroscopic guidance.

In some embodiments, the implant 70 can be located, prior to release, above the mitral annulus 1106, in line with the mitral annulus 1106, or below the mitral annulus 1106. In some embodiments, the implant 70 can be located, prior to expansion, fully above the mitral annulus 1106, in line with the mitral annulus 1106, just below the mitral annulus 1106, or fully below the mitral annulus 1106. In some embodiments, the implant 70 can be located, prior to expansion, partially above the mitral annulus 1106, in line with the mitral annulus 1106, or partially below the mitral annulus 1106. In some embodiments, a pigtail catheter can be introduced into the heart to perform a ventriculogram for proper viewing.

In some embodiments, the position of the mitral plane and the height of any papillary muscles on the fluoroscopy monitor can be marked to indicate an example target landing zone. If needed, the delivery system 10 can be unflexed, reduced in rotation, and retracted to reduce tension on the delivery system 10 as well as reduce contact with the left ventricular wall, the left atrial wall, and/or the mitral annulus 1106.

Further, the delivery system 10 can be positioned to be coaxial to the mitral annulus 1106, or at least as much as possible, while still reducing contact with the left ventricular wall, the left atrial wall, and/or the mitral annulus 1106 and reducing delivery system tension. An echo probe can be positioned to view the anterior mitral leaflet (AML), the posterior mitral leaflet (PML) (leaflets 1108), mitral annulus 1106, and outflow tract. Using fluoroscopy and echo imaging, the implant 70 can be confirmed to be positioned at a particular depth and coaxiality with the mitral annulus 1106.

Afterwards, the outer sheath assembly 22 can be retracted to expose the ventricular anchors 80, thereby releasing them. In some embodiments, once exposed, the outer sheath assembly 22 can be reversed in direction to relieve tension on the outer sheath assembly 22. In some embodiments, reversing the direction could also serve to partially or fully capture the implant 70.

The distal anchors 80 can be released in the left atrium 1078. Further, the proximal anchors 82, if included in the implant 70, are not yet exposed. Moreover, the body of the implant 70 has not undergone any expansion at this point. However, in some embodiments, one or more of the distal anchors 80 can be released in either the left atrium 1078 (e.g., super-annular release) or generally aligned with the mitral valve annulus 1106 (e.g., intra-annular release), or just below the mitral valve annulus 1106 (e.g., sub-annular release). In some embodiments, all of the distal anchors 80 can be released together. In other embodiments, a subset of the distal anchors 80 can be released while at a first position and another subset of the distal anchors 80 can be released while at a second position. For example, some of the distal anchors 80 can be released in the left atrium 1078 and some of the distal anchors 80 can be released while generally aligned with the mitral valve annulus 1106 or just below the mitral valve annulus 1106.

If the distal anchors 80 are released “just below” the mitral valve annulus 1106, the may be released at 1 inch, ¾ inch, ½ inch, ¼ inch, ⅛ inch, 1/10 inch or 1/20 inch below the mitral valve annulus 1106. In some embodiments, the distal anchors 80 the may be released at less than 1 inch, ¾ inch, ½ inch, ¼ inch, ⅛ inch, 1/10 inch or 1/20 inch below the mitral valve annulus 1106. This may allow the distal anchors 80 to snake through the chordae upon release. This can advantageously allow the implant 70 to slightly contract when making the sharp turn down toward the mitral valve. In some embodiments, this may eliminate the need for a guide wire assisting to cross the mitral valve. In some embodiments, the guide wire may be withdrawn into the delivery system 10 before or following release of the distal anchors 80.

In some embodiments, the distal anchors 80 can be released immediately after crossing the septum, and then the final trajectory of the delivery system 10 can be determined. Thus, the delivery system 10 can cross the septum, release the ventricular anchors 80, establish a trajectory, and move into the left ventricle to capture the leaflets.

As discussed in detail above, upon release from the delivery system 10, the distal anchors 80 can flip from extending distally to extending proximally. This flip can be approximately 180°. Accordingly, in some embodiments, the distal anchors 80 can be flipped in either the left atrium 1078 (e.g., super-annular flip), generally aligned with the mitral valve annulus 1106 (e.g., intra-annular flip), or just below the mitral valve annulus 1106 (e.g., sub-annular flip). The proximal anchors 82, if any, can remain within the delivery system 10. In some embodiments, all of the distal anchors 80 can be flipped together. In other embodiments, a subset of the distal anchors 80 can be flipped while at a first position and another subset of the distal anchors 80 can be released while at a second position. For example, some of the distal anchors 80 can be flipped in the left atrium 1078 and some of the distal anchors 80 can be flipped while generally aligned with the mitral valve annulus 1106 or just below the mitral valve annulus 1106.

In some embodiments, the distal anchors 80 may be positioned in line with the annulus 1106 or just below the annulus 1106 in the non-flipped position. In some embodiments, the distal anchors 80 may be position in line with the annulus 1106 or just below the annulus 1106 in the flipped position. In some embodiments, prior to flipping the distalmost portion of the implant 70 can be located within or below the mitral valve annulus 1106, such as just below the mitral valve annulus 1106. However, flipping the anchors can cause, without any other movement of the delivery system 10, the distalmost portion of the implant 70/anchors 80 to move upwards, moving it into the left atrium 1078 or moving it in line with the mitral annulus 1106. Thus, in some embodiments the distal anchors 80 can begin flipping at the annulus 1106 but be fully within the left atrium 1078 upon flipping. In some embodiments the distal anchors 80 can begin flipping below the annulus 1106 but be fully within the annulus 1106 upon flipping.

In some embodiments, the distal anchors 80 can be proximal (e.g., toward the left atrium 1078) of a free edge of the mitral leaflets 1108 upon release and flipping. In some embodiments, the distal anchors 80 can be aligned with (e.g., toward the left atrium 1078) a free edge of the mitral leaflets 1108 upon release and flipping. In some embodiments, the distal anchors 80 can be proximal (e.g., toward the left atrium 1078) of a free edge of the mitral valve annulus 1106 upon release and flipping. In some embodiments, the distal anchors 80 can be aligned with (e.g., toward the left atrium 1078) a free edge of the mitral valve annulus 1106 upon release and flipping.

Thus, in some embodiments the distal anchors 80 can be released/flipped above where the chordae 1110 attach to the free edge of the native leaflets 1108. In some embodiments the distal anchors 80 can be released/flipped above where some the chordae 1110 attach to the free edge of the native leaflets 1108. In some embodiments the distal anchors 80 can be released/flipped above where all the chordae 1110 attach to the free edge of the native leaflets 1108. In some embodiments, the distal anchors 80 can be released/flipped above the mitral valve annulus 1106. In some embodiments, the distal anchors 80 can be released/flipped above the mitral valve leaflets 1108. In some embodiments, the distal anchors 80 can be released/flipped generally in line with the mitral valve annulus 1106. In some embodiments, the distal anchors 80 can be released/flipped generally in line with the mitral valve leaflets 1108. In some embodiments, the tips of the distal anchors 80 can be released/flipped generally in line with the mitral valve annulus 1106. In some embodiments, the tips of the distal anchors 80 can be released/flipped generally in line with the mitral valve leaflets 1108. In some embodiments the distal anchors 80 can be released/flipped below where some the chordae 1110 attach to the free edge of the native leaflets 1108. In some embodiments the distal anchors 80 can be released/flipped below where all the chordae 1110 attach to the free edge of the native leaflets 1108. In some embodiments, the distal anchors 80 can be released/flipped below the mitral valve annulus 1106. In some embodiments, the distal anchors 80 can be released/flipped below the mitral valve leaflets 1108.

Once the distal anchors 80 are released and flipped, the delivery system 10 can be translated towards the left ventricle 1080 through the mitral valve annulus 1106 so that the distal anchors 80 enter the left ventricle 1080. In some embodiments, the distal anchors 80 can compress when passing through the mitral valve annulus 1106. In some embodiments, the implant 70 can compress when passing through the mitral valve annulus 1106. In some embodiments, the implant 70 does not compress when it passes through the mitral annulus 1106. The distal anchors 80 can be delivered anywhere in the left ventricle 1080 between the leaflets 1108 and the papillary heads.

In some embodiments, the distal anchors 80 are fully expanded prior to passing through the mitral valve annulus 1106. In some embodiments, the distal anchors 80 are partially expanded prior to passing through the mitral valve annulus 1106 and continued operation of the delivery system 10 can fully expand the distal anchors 80 in the left ventricle 1080.

When the distal anchors 80 enter the left ventricle 1080, the distal anchors 80 can pass through the chordae 1110 and move behind the mitral valve leaflets 1108, thereby capturing the leaflets 1108. In some embodiments, the distal anchors 80 and/or other parts of the implant 70 can push the chordae 1110 and/or the mitral valve leaflets 1108 outwards.

Thus, after release of the distal anchors 80, the delivery system 10 can then be repositioned as needed so that the ends of the left distal anchors 80 are at the same level of the free edge of the native mitral valve leaflets 1108. The delivery system 10 can also be positioned to be coaxial to the mitral annulus 1106 if possible while still reducing contact with the left ventricular wall, the left atrial wall, and/or the annulus 1106.

In some embodiments, only the distal anchors 80 are released in the left atrium 1078 before the implant 70 is move to a position within, or below, the annulus. In some alternate embodiments, the distal end of the implant 70 can be further expanded in the left atrium 1078. Thus, instead of the distal anchors 80 flipping and no portion of the implant 70 body expanding, a portion of the implant 70 can be exposed and allowed to expand in the left atrium 1078. This partially exposed implant 70 can then be passed through the annulus 1106 into the left ventricle 1080. Further, the proximal anchors, if any, can be exposed. In some embodiments, the entirety of the implant 70 can be expanded within the left atrium 1078.

To facilitate passage through the annulus 1106, the delivery system 10 can include a leader element (not shown) which passes through the annulus 1106 prior to the implant 70 passing through the annulus 1106. For example, the leader element can include an expandable member, such as an expandable balloon, which can help maintain the shape, or expand, the annulus 1106. The leader element can have a tapered or rounded shape (e.g., conical, frustoconical, semispherical) to facilitate positioning through and expansion of the annulus 1106. In some embodiments, the delivery system 10 can include an engagement element (not shown) which can apply a force on the implant 70 to force the implant 70 through the annulus 1106. For example, the engagement element can include an expandable member, such as an expandable balloon, positioned within or above the implant 70.

In some embodiments, to facilitate passage through the annulus 1106, a user can re-orient the implant 70 prior to passing the implant 70 through the annulus 1106. For example, a user can re-orient the implant 70 such that it passes through the annulus 1106 sideways.

However, if only the distal anchors 80 are flipped, and no other expansion occurs, the prosthesis can be partially expanded in the ventricle 1080. Thus, when the implant 70 is in the proper location, the distal end can be allowed to expand to capture the leaflets 1108. If the distal end is already expanded, no more expansion may take place or the distal end can be further expanded.

Further, the PML and AML 1106 can be captured, for example by adjusting the depth and angle of the implant 70. If a larger prosthesis diameter is needed to capture the leaflets 1108, the outer sheath assembly 22 can be retracted until the desired diameter of the implant 70 is achieved. Capture of the leaflets 1108 can be confirmed through echo imaging. In some embodiments, a user can confirm that the implant 70 is still in the appropriate depth and has not advanced into the left ventricle 1080. The position can be adjusted as needed.

In some embodiments, once the distal anchors 80 enter the left ventricle 1080 the system 10 can be pulled backwards (e.g., towards the left atrium 1078) to fully capture the leaflets 1108. In some embodiments, the system 10 does not need to be pulled backwards to capture the leaflets 1108. In some embodiments, systolic pressure can push the leaflets 1108 upwards to be captured by the distal anchors 80. In some embodiments, systolic pressure can push the entire implant 70 up towards the mitral annulus 1106 after the leaflets 1108 are captured and the implant 70 is fully or partially released. In some embodiments, a user can rotate the delivery system 10 and/or implant 70 prior to and/or while pulling the delivery system 10 backwards. In some instances, this can beneficially engage a greater number of chordae tendineae.

The outer sheath assembly 22 can be further retracted to fully expand the prosthesis. Once the implant 70 is fully exposed, the delivery system 10 can be maneuvered to be coaxial and height relative to the mitral annulus 1106, such as by flexing, translating, or rotating the delivery system 10. As needed, the implant 70 can be repositioned to capture the free edge of the native mitral valve leaflets 1108. Once full engagement of the leaflets 1108 is confirmed, the implant 70 can be set perpendicular (or generally perpendicular) to the mitral annular plane.

Following, the mid shaft assembly 21 can be withdrawn. The mid shaft assembly 21 can then be reversed in direction to relieve any tension on the delivery system 10.

Below is a discussion of proximal anchors 82, though some embodiments of the implant 70 may not include them. In some embodiments, proximal anchors 82 may not be released from the system 10 until the distal anchors 80 have captured the leaflets 1108. In some embodiments, proximal anchors 82 may be released from the system 10 prior to the distal anchors 80 capturing the leaflets 1108. In some embodiments, the proximal anchors 82 can be released when the distal anchors 80 are super or intra annular and the expanded implant 70 (either partially or fully expanded) can be translated through the mitral annulus 1106. In some embodiments, the proximal anchors 82 could be released when the distal anchors 80 are sub-annular and the entire implant 70 can be pulled up into the left atrium 1078 such that the proximal anchors 82 are supra-annular prior to release. In some embodiments, the proximal anchors 82 could be intra-annular prior to release and the systolic pressure could push the implant 70 atrially such that the proximal anchors 82 end up supra-annular.

After, the leaflet capture and positioning of the implant 70 can be confirmed, along with the relatively perpendicular position with respect to the mitral annular plane. In some embodiments, the nose cone 28 can then be withdrawn until it is within the implant 70. The mid shaft assembly 21 can be further retracted until the implant 70 is released from the delivery system 10. Proper positioning of the implant 70 can be confirmed using TEE and fluoroscopic imaging.

Following, the delivery system 10 can be centralized within the implant 70. The nose cone 28 and delivery system 10 can then be retracted into the left atrium 1078 and removed.

This intra-super annulus release can have a number of advantages. For example, this allows the distal anchors 80 to be properly aligned when contacting the chordae 1110. If the distal anchors 80 were released in the left ventricle 1080, this could cause misalignment or damage to heart tissue, such as the leaflets 1108 or chordae 1110.

In an alternate delivery approach, the delivery system 10 can be translated into the left ventricle 1080 prior to release of the implant 70. Thus, the distal end of the implant 70, and thus the distal anchors 80, can be released and flipped partially, or fully within the left ventricle 1080. Accordingly, in some embodiments the anchors 82 can be released/flipped below the mitral annulus 1106, just below the mitral annulus 1106, and/or below the free edges of the leaflets 1108. Further, the anchors 82 can be released above the papillary heads. Similar methodology as discussed above can then be used to properly position the implant 70 and remove the delivery system 10 to deliver the implant 70. Further, in some embodiments the distal anchors 80 can be released without expanding the prosthesis initially in the ventricle 1080.

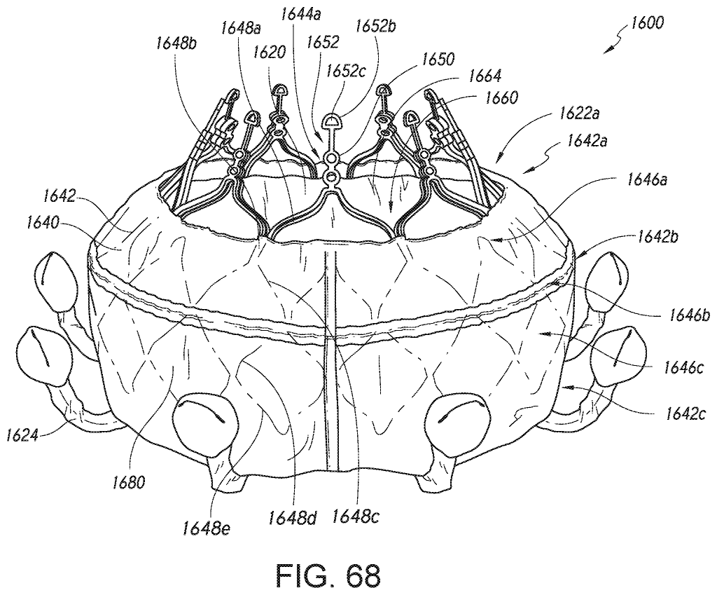

Although many of the systems and methods disclosed herein have been discussed in regard to implantation of a prosthetic mitral valve implant, it is understood that the systems and methods may be utilized to deliver a variety of implants, including implants for repair of a heart valve. For example, other types of heart valve implants that may be utilized are shown in FIGS. 68-69, among other types of implants (e.g., aortic valve implants and other repair implants).

The methods and systems disclosed herein may in certain embodiments not be limited to delivery of implants, but may extend to any medical intervention or insertion into a patient's body, which may include performing a medical procedure within the body. The methods and systems disclosed herein may be utilized in general use of a catheter as desired. For example, the handle shown in FIGS. 35 and 37 and components disclosed therein may comprise a general catheter handle in certain embodiments. Further, the configuration of the delivery apparatus may be modified in other embodiments. For example, for an aortic valve delivery apparatus, the configuration of the implant retention area and other features of the delivery apparatus may be modified.

With reference next to FIG. 68-69, an alternative embodiment of an implant 1600 in an expanded configuration is illustrated. The implant 1600 can include an inner frame 1620, an outer frame 1640, a valve body 1660, and one or more skirts, such as an outer skirt 1680 and an inner skirt 1690.

With reference first to the outer frame 1640 illustrated in FIG. 68-69, the outer frame 1640 can be attached to the inner frame 1620 using any known fasteners and/or techniques. Although the outer frame 1640 is illustrated as a separate component from the inner frame 1620, it is to be understood that the frames 1620, 1640 can be unitarily or monolithically formed.

As shown in the illustrated embodiment, the outer frame 1640 can include an outer frame body 1642. The outer frame body 1642 can have an upper region 1642a, an intermediate region 1642b, and a lower region 1642c. At least a portion of the upper region 1642a of the outer frame body 1642 can be sized and/or shaped to generally match the size and/or shape of an upper region 1622a of the inner frame 1620. As shown in the illustrated embodiment, the upper region 1642a of the outer frame body 1642 can include one or more struts which generally match the size and/or shape of struts of the inner frame 1620. This can locally reinforce a portion of the implant 1600 by effectively increasing the wall thickness of the combined struts.

When in an expanded configuration such as in a fully expanded configuration, the intermediate region 1642b and the lower region 1642c can have a diameter which is larger than the diameter of the upper region 1642a. The upper region 1642a of the outer frame body 1642 can have a decreasing diameter from a lower end to an upper end such that the upper region 1642a is inclined or curved radially inwards towards the longitudinal axis of the implant 1600. Although the outer frame body 1642 has been described and illustrated as being cylindrical or having circular cross-sections, it is to be understood that all or a portion of the outer frame body 1642 can be have a non-circular cross-section such as, but not limited to, a D-shape, an oval or an otherwise ovoid cross-sectional shape.

With continued reference to the outer frame 1640 illustrated in FIG. 68-69, the outer frame body 1642 can include a plurality of struts with at least some of the struts forming cells 1646a-c. Any number of configurations of struts can be used, such as rings of undulating struts shown forming ellipses, ovals, rounded polygons, and teardrops, but also chevrons, diamonds, curves, and various other shapes.

The upper row of cells 1646a can have an irregular octagonal shape such as a “heart” shape. This additional space can beneficially allow the outer frame 1640 to retain a smaller profile when crimped. The cell 1646a can be formed via a combination of struts. As shown in the illustrated embodiment, the upper portion of cells 1646a can be formed from a set of circumferentially-expansible struts 1648a having a zig-zag or undulating shape forming a repeating “V” shape. The struts 1648a can extend radially outwardly from an upper end to a lower end. These struts can generally match the size and/or shape of struts of the inner frame 1620.

The middle portion of cells 1646a can be formed from a set of struts 1648b extending downwardly from bottom ends of each of the “V” shapes. The struts 1648b can extend radially outwardly from an upper end to a lower end. The portion of the cells 1646a extending upwardly from the bottom end of struts 1648b may be considered to be a substantially non-foreshortening portion of the outer frame 1640.

The lower portion of cells 1646a can be formed from a set of circumferentially-expansible struts 1648c having a zig-zag or undulating shape forming a repeating “V” shape. As shown in the illustrated embodiment, the struts 1648c can incorporate a curvature such that the lower end of struts 1648c extend more parallel with the longitudinal axis than the upper end of the struts 1648c. One or more of the upper ends or tips of the circumferentially-expansible struts 1648c can be a “free” apex which is not connected to a strut. For example, as shown in the illustrated embodiment, every other upper end or tip of circumferentially-expansible struts 1648b is a free apex. However, it is to be understood that other configurations can be used. For example, every upper apex along the upper end can be connected to a strut.

The middle and/or lower rows of cells 1646b-c can have a different shape from the cells 1646a of the first row. The middle row of cells 1646b and the lower row of cells 1646c can have a diamond or generally diamond shape. The diamond or generally diamond shape can be formed via a combination of struts.

The upper portion of cells 1646b can be formed from the set of circumferentially-expansible struts 1648c such that cells 1646b share struts with cells 1646a. The lower portion of cells 1646b can be formed from a set of circumferentially-expansible struts 1648d. As shown in the illustrated embodiment, one or more of the circumferentially-expansible struts 1648d can extend generally in a downward direction generally parallel to the longitudinal axis of the outer frame 1640.

The upper portion of cells 1646c can be formed from the set of circumferentially-expansible struts 1648d such that cells 1646c share struts with cells 1646b. The lower portion of cells 1646c can be formed from a set of circumferentially-expansible struts 1648e. Circumferentially-expansible struts 1648e can extend generally in a downward direction.

As shown in the illustrated embodiment, there can be a row of nine cells 1646a and a row of eighteen cells 1646b-c. While each of the cells 1646a-c are shown as having the same shape as other cells 1646a-c of the same row, it is to be understood that the shapes of cells 1646a-c within a row can differ. Moreover, it is to be understood that any number of rows of cells can be used and any number of cells may be contained in the rows.

As shown in the illustrated embodiment, the outer frame 1640 can include a set of eyelets 1650. The upper set of eyelets 1650 can extend from an upper region 1642a of the outer frame body 1642. As shown, the upper set of eyelets 1650 can extend from an upper portion of cells 1646a, such as the upper apices of cells 1646a. The upper set of eyelets 1650 can be used to attach the outer frame 1640 to the inner frame 1620. For example, in some embodiments, the inner frame 1620 can include one or more eyelets which correspond to the eyelets 1650. In such embodiments, the inner frame 1620 and outer frame 1640 can be attached together via eyelets 1650 and corresponding eyelets on the inner frame 1620. For example, the inner frame 1620 and outer frame 1640 can be sutured together through said eyelets or attached via other means, such as mechanical fasteners (e.g., screws, rivets, and the like).

As shown, the set of eyelets 1650 can include two eyelets extending in series from each “V” shaped strut. This can reduce the likelihood that the outer frame 1640 twists along an axis of the eyelet. However, it is to be understood that some “V” shaped struts may not include an eyelet. Moreover, it is to be understood that a fewer or greater number of eyelets can extend from a “V” shaped strut.

The outer frame 1640 can include a set of locking tabs 1652 extending from at or proximate an upper end of the upper region 1642a. As shown, the locking tabs 1652 can extend upwardly from the set of eyelets 1650. The outer frame 1640 can include twelve locking tabs 1652, however, it is to be understood that a greater number or lesser number of locking tabs can be used. The locking tabs 1652 can include a longitudinally-extending strut 1652a. At an upper end of the strut 1652a, the locking tab 1652 can include an enlarged head 1652b. As shown, the enlarged head 1652b can have a semi-circular or semi-elliptical shape forming a “mushroom” shape with the strut 1652a. The locking tab 1652 can include an eyelet 1652c which can be positioned through the enlarged head 1652b. It is to be understood that the locking tab 1652 can include an eyelet at other locations, or can include more than a single eyelet.

The locking tab 1652 can be advantageously used with multiple types of delivery systems. For example, the shape of the struts 1652a and the enlarged head 1652b can be used to secure the outer frame 1640 to a “slot” based delivery system, such as the inner retention member 40 described above. The eyelets 1652c and/or eyelets 1650 can be used to secure the outer frame 1640 to a “tether” based delivery system such as those which utilize sutures, wires, or fingers to control delivery of the outer frame 1640 and the implant 1600. This can advantageously facilitate recapture and repositioning of the outer frame 1640 and the implant 1600 in situ.

The outer frame 1640, such as the outer frame body 1642 can be used to attach or secure the implant 1600 to a native valve, such as a native mitral valve. For example, the intermediate region 1642b of the outer frame body 1642 and/or the outer anchoring feature 1644 can be positioned to contact or engage a native valve annulus, tissue beyond the native valve annulus, native leaflets, and/or other tissue at or around the implantation location during one or more phases of the cardiac cycle, such as systole and/or diastole. As another example, the outer frame body 1642 can be sized and positioned relative to the inner frame anchoring feature 1624 such that tissue of the body cavity positioned between the outer frame body 1642 and the inner frame anchoring feature 1624, such as native valve leaflets and/or a native valve annulus, can be engaged or pinched to further secure the implant 1600 to the tissue. As shown, the inner frame anchoring feature 1624 includes nine anchors; however, it is to be understood that a fewer or greater number of anchors can be used. In some embodiments, the number of individual anchors can be chosen as a multiple of the number of commissures for the valve body 1660. For example, for a valve body 1660 have three commissures, the inner frame anchoring feature 1624 can have three individual anchors (1:1 ratio), six individual anchors (2:1 ratio), nine individual anchors (3:1 ratio), twelve individual anchors (4:1 ratio), fifteen individual anchors (5:1 ratio), or any other multiple of three. In some embodiments, the number of individual anchors does not correspond to the number of commissures of the valve body 1660.

With continued reference to the prosthesis 1600 illustrated in FIGS. 68-69, the valve body 1660 is attached to the inner frame 1620 within an interior of the inner frame body 1620. The valve body 1660 functions as a one-way valve to allow blood flow in a first direction through the valve body 1660 and inhibit blood flow in a second direction through the valve body 1660.

The valve body 1660 can include a plurality of valve leaflets 1662, for example three leaflets 1662, which are joined at commissures. The valve body 1660 can include one or more intermediate components 1664. The intermediate components 1664 can be positioned between a portion of, or the entirety of, the leaflets 1662 and the inner frame 1620 such that at least a portion of the leaflets 1662 are coupled to the frame 1620 via the intermediate component 1664. In this manner, a portion of, or the entirety of, the portion of the valve leaflets 1662 at the commissures and/or an arcuate edge of the valve leaflets 1662 are not directly coupled or attached to the inner frame 1620 and are indirectly coupled or “float” within the inner frame 1620.

With reference next to the outer skirt 1680 illustrated in FIGS. 68-69, the outer skirt 1680 can be attached to the inner frame 1620 and/or outer frame 1640. As shown, the outer skirt 1680 can be positioned around and secured to a portion of, or the entirety of, the exterior of the outer frame 1640. The inner skirt 1690 can be attached to the valve body 1660 and the outer skirt 1680. As shown in FIG. 69, a first end of the inner skirt 1690 can be coupled to the valve body 1660 along portions of the valve body 1660 which are proximate the inner frame 1620. A second end of the inner skirt 1690 can be attached to the lower region of the outer skirt 1680. In so doing, a smooth surface can be formed along under each of the leaflets. This can beneficially enhance hemodynamics by allowing blood to more freely circulate and reducing areas of stagnation.

Although the implant 1600 has been described as including an inner frame 1620, an outer frame 1640, a valve body 1660, and skirts 1680, 1690, it is to be understood that the implant 1600 need not include all components. For example, in some embodiments, the implant 1600 can include the inner frame 1620, the outer frame 1640, and the valve body 1660 while omitting the skirt 1680. Moreover, although the components of the implant 1600 have been described and illustrated as separate components, it is to be understood that one or more components of the implant 1600 can be integrally or monolithically formed. For example, in some embodiments, the inner frame 1620 and the outer frame 1640 can be integrally or monolithically formed as a single component.

The systems, apparatuses, and methods disclosed herein may be utilized in a recapture of an implant that has been fully or partially deployed. For example, in the methods disclosed in regard to FIG. 61-67B, the capsule 106 may be configured to slide distally for recapture of an implant 70 that has been partially or possibly fully deployed. The capsule 106 may accordingly move distally to pull all or a portion of the implant 70 back into the capsule 106 to recapture the implant 70.

With reference to FIG. 64, for example, an implant 70 is shown to be partially deployed from the capsule 106. The arms of the implant 70, in the form of distal anchors 80, have extended outward from the capsule 106 and have bent in a proximal direction relative to their orientation when positioned within the capsule 106 (for example as shown in FIG. 2A). Notably, in this configuration, the distal anchors 80 may extend between chordae 1110 as shown for example in FIG. 62.

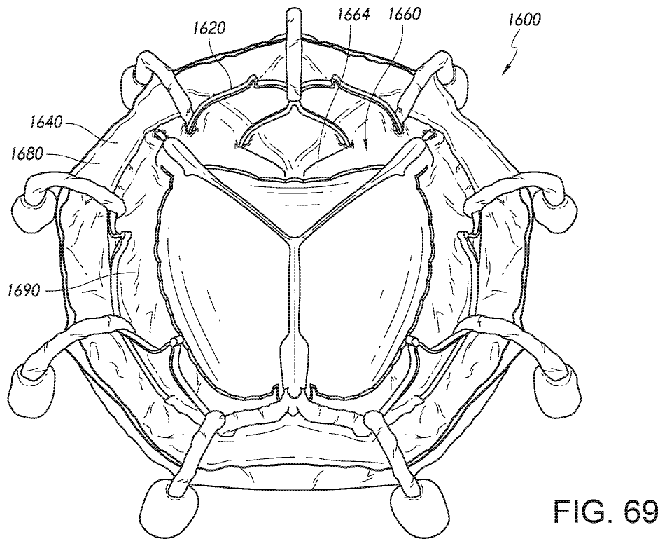

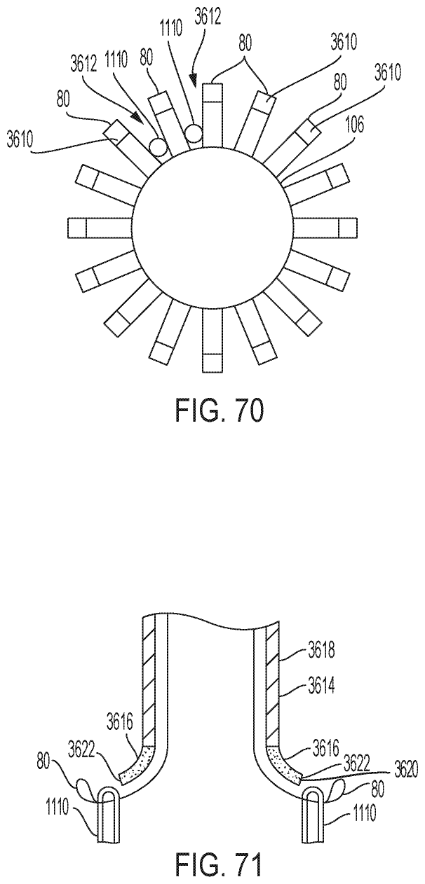

FIG. 70 illustrates a cross sectional view of the capsule 106 in a plane transverse to the axis of the capsule 106. The distal anchors 80 are shown to extend radially outward from the outer surface of the capsule 106 as shown in a perspective view in FIG. 64 for example. The tips 3610 of the distal anchors 80 extend proximally as shown in a perspective view in FIG. 64 for example.

As shown in FIG. 70, the chordae 1110 may be positioned within a narrow spacing 3612 between adjacent distal anchors 80. If the capsule 106 is advanced distally to recapture the implant 70 after full or partial deployment of the implant 70 then the distal anchors 80 may pinch the one or more chordae 1110 positioned within the narrow spacing 3612. As the distal anchors 80 are pulled into the capsule 106, the chordae 1110 may press against the capsule 106 at a radial distance that cause the chordae 1110 to pinch within the narrow spacing 3612. This possible complication may produce injury to the chordae 1110 including possibly severing one or more chordae 1110.

FIG. 71 illustrates a side cross sectional view of an embodiment of a capsule 3614 that has a distal end 3616 that is configured to expand radially outward. The capsule 3614 may otherwise be configured similarly as the capsule 106 or any other embodiment of capsule disclosed herein. For example, the capsule 3614 may be configured to surround an implant retention area.

The distal end 3616 may be configured to bend radially outward. The distal end 3616 as shown in FIG. 71 may be configured to bend radially outward relative to a proximal portion 3618 of the capsule 3614. The distal end 3616 may include an opening 3620 for an implant 70 to be deployed from.

The distal end 3616 may be configured to flare outward relative to the proximal portion 3618 of the capsule 3614. The distal end 3616 may include a contact surface 3622 that is configured to contact and apply a force to the chordae 1110 to wipe any chordae 1110 off of the anchors 80 as the anchors 80 are being retracted back into the capsule 3614 during recapture. The flaring of the distal end 3616 may allow the contact surface 3622 to contact the chordae 1110 at a greater radial position than the position of the chordae 1110 shown in FIG. 70, and as such the chordae 1110 may be less likely to be pinched within a narrow spacing 3612 between the anchors 80. As such, the possibility for damage to the chordae 1110 may be reduced during a recapture of the implant 70.

The distal end 3616 may be configured to flare outward passively, which may be caused by the outward force of the anchors 80 upon the interior surface of the distal end 3616. In other embodiments, the distal end 3616 may be configured to be controlled to flare the distal end 3616 in a desired manner.

The distal end 3616 may be made of a flexible material, which may comprise an elastomer or another form of flexible material. The distal end 3616 may be configured to be pliable, and as such may form a large contact surface area against the anchors 80 to wipe the chordae 1110 off of the anchors 80. The distal end 3616 may be pliable to pass within spacings between the anchors 80. The distal end 3616 may be resilient to return back towards an initial shape of the distal end 3616 upon recapture of the implant 70, and to resist permanent deformation during flaring of the distal end 3616. Other configurations of distal ends 3616 may be utilized as desired.

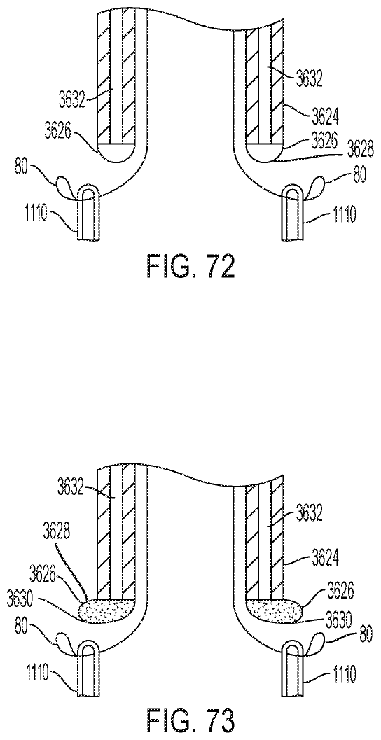

FIG. 72 illustrates a side cross sectional view of an embodiment of a capsule 3624 that has a distal end 3626 that is configured to expand radially outward. The capsule 3624 may otherwise be configured similarly as the capsule 106 or any other embodiment of capsule disclosed herein. For example, the capsule 3624 may be configured to surround an implant retention area. The distal end 3626 of the capsule 3624 may be configured to expand radially outward by being inflated radially outward.

The distal end 3626 may include an inflatable body 3628 that is configured to inflate. The inflatable body 3628 may comprise a balloon or other form of inflatable body that is configured to inflate to cause the distal end 3626 to flare radially outward. The distal end 3626 may include a contact surface 3630 (marked in FIG. 73) that operates similarly as the contact surface 3622 shown in FIG. 71. As such, the contact surface 3630 of the distal end 3626 may be configured to contact and wipe any chordae 1110 off of the anchors 80 as the anchors 80 are being retracted back into the capsule 3624 during recapture. The flaring of the distal end 3626 may allow the contact surface 3630 to contact the chordae 1110 at a greater radial position than the position of the chordae 1110 shown in FIG. 70, and as such the chordae 1110 may be less likely to be pinched within a narrow spacing 3612 between anchors 80. As such, the possibility for damage to the chordae 1110 may be reduced during a recapture of the implant 70. The inflatable body 3628 may be compliant, to allow a portion of the inflatable body 3628 to be positioned between the anchors 80.

The distal end 3626 may be configured to vary in size. FIG. 72 illustrates the distal end 3626 in an unexpanded, uninflated, or undeployed configuration, and FIG. 73 illustrates the distal end 3626 in an expanded, inflated, or deployed configuration. The distal end 3626 in the expanded, inflated, or deployed configuration has a greater size and a greater radial extent than in the unexpanded, uninflated, or undeployed configuration. At least one inflation conduit 3632 may extend along the elongate shaft of the delivery system and be configured to inflate the inflatable body 3628 of the distal end 3626 with fluid or other substances for inflating the distal end 3626. The inflation conduits 3632 may be controlled from a proximal portion of the delivery system to inflate or deflate the distal end 3626 and thus control the size and radial extent of the distal end 3626.

The distal end 3626 may be configured to be expanded, inflated, or deployed at a desired time for recapture of the implant, and may then be unexpanded, deflated, or undeployed following recapture, or following a time for withdrawal of the delivery system from a portion of the patient's body. As such, the distal end 3626 may move from a configuration shown in FIG. 73 back to a configuration shown in FIG. 72. Other configurations of distal ends 3626 may be utilized as desired.

The embodiments disclosed herein may be utilized in a method including deploying an elongate shaft to a location within a patient's body, the elongate shaft including a capsule surrounding an implant retention area retaining an implant for implantation within the patient's body. The capsule may be moved proximally to expose a portion of the implant within the patient's body. The capsule may then be moved distally to recapture a portion of the implant within the patient's body and to pass a distal end 3616, 3626 of the capsule that is expanded radially outward over the portion of the implant that is recaptured. The portion of the implant that is recaptured may include arms of the implant, which may comprise anchors of the implant. The distal end 3616, 3626 of the capsule may be positioned between the arms of the implant, to allow the distal end 3616, 3626 to press against chordae between the arms of the implant. The method may include pushing chordae off of the arms of the implant with the distal end 3616, 3626 of the capsule.

The embodiments of distal ends disclosed in FIGS. 71-73 may be utilized solely or with any embodiment of a delivery system or other system, apparatus, or method disclosed herein.

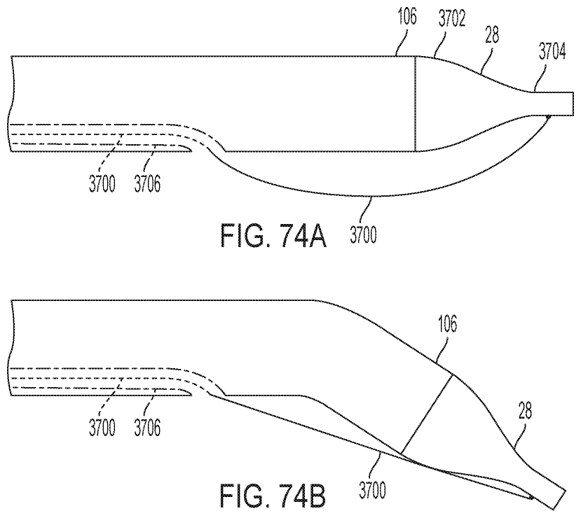

FIGS. 74A and 74B illustrate an embodiment in which a delivery system may utilize a pull tether 3700 that is coupled to a portion of the elongate shaft of the delivery system at or distal the implant retention area and is configured to deflect the distal end of the elongate shaft. Referring to FIG. 2B, for example, the implant retention area 16 is shown to be surrounded by the capsule 106 and has the nose cone shaft 27 extending within the implant retention area 16. The steerable rail assembly 20, however, is positioned proximal of the implant retention area 16. As such, upon bending of the steerable rail assembly 20, the nose cone 28 at the distal end of the elongate shaft follows the bend created by the steerable rail assembly 20 at a position proximal of the implant retention area 16. The embodiment of FIGS. 74A and 74B, however, couples the pull tether 3700 at a position that is at or distal the implant retention area 16. As such, greater torque and control of the distal end of the delivery system may result.

In the embodiment shown in FIGS. 74A and 74B, for example, a distal end of the pull tether 3700 is coupled to the nose cone 28. The distal end of the pull tether 3700 may be positioned at a distal portion of the nose cone 28. For example, the nose cone 28 may include a proximal portion 3702 and a distal portion 3704 and the pull tether 3700 may be coupled to the distal portion 3704 of the nose cone 28. The distal coupling position of the pull tether 3700 may increase the torque that is provided upon the nose cone 28. In other embodiments, other coupling positions may be utilized, such as on the proximal portion 3702 of the nose cone 28.

The pull tether 3700 may have a variety of forms and may comprise a pull wire as disclosed herein or another form of tether. The pull tether 3700 as shown in FIGS. 74A and 74B may have at least a portion that extends external to the elongate shaft. Such a configuration may allow for increased torque upon the distal end of the elongate shaft. At least a portion of the pull tether 3700 may then extend within a channel 3706 internal to the elongate shaft such that the pull tether 3700 does not extend entirely external to the elongate shaft. For example, as shown in FIGS. 74A and 74B, the pull tether 3700 may extend external to the capsule 106. The pull tether 3700 may be pulled and released to control the deflection of the distal end of the elongate shaft.

The distal end of the pull tether 3700 may be coupled to other locations as desired. For example, referring to FIG. 2B, the distal end of the pull tether 3700 may be coupled to the interior nose cone shaft 27 that is coupled to the nose cone 28. The implant retention area 16 may include a proximal portion 3708 and a distal portion 3710, and the pull tether 3700 may be coupled to a portion of the elongate shaft within the distal portion 3710 of the implant retention area 16. As such, the pull tether 3700 may provide torque to the distal end of the elongate shaft that is in addition to any torque provided by the steerable rail assembly 20 that is positioned proximal of the implant retention area 16 and is configured to deflect a portion of the elongate shaft that is positioned proximal of the implant retention area 16.

The innermost assembly of the elongate shaft, as shown in FIG. 2B as the nose cone assembly 31 may accordingly be steerable. The pull tether 3700 may extend proximally from the distal end of the elongate shaft for manipulation at a proximal end of the delivery system to control the deflection of the distal end of the delivery system.

The coupling of the pull tether 3700 to a portion of the elongate shaft of the delivery system at or distal the implant retention area may allow for greater control of the distal end of the elongate shaft. As such, the nose cone 28 forming the tip of the elongate shaft and the nose cone shaft 27 may have additional directions and degrees of flex than provided by the steerable rail assembly 20. Further, the pull tether 3700 may provide for flex distal of the steerable rail assembly 20. The pull tether 3700 may thus allow for tighter turns and greater precision of control of the distal end of the elongate shaft. In embodiments, the pull tether 3700 may be configured to allow for flex in a same plane as provided by the steerable rail assembly 20, or in a different plane.

The pull tether 3700 may also allow the use of a guide wire to be eliminated if desired. For example, the nose cone shaft 27 may lack an internal lumen for the guide wire. The additional control provided by the pull tether 3700 may allow the distal end of the elongate shaft to be controlled such that a guide wire is not needed to direct the distal end of the elongate shaft. In other embodiments, a guide wire may be utilized. The configuration of the pull tether 3700 may be varied in other embodiments.

In embodiments herein, the delivery system may include two elongate shafts, or at least two elongate shafts, that may be utilized in combination to deliver an implant to a location within a patient's body. A first elongate shaft may be steerable, and another or second elongate shaft may include an implant retention area that is configured to retain the implant and may include a deployment mechanism. Each elongate shaft may include a respective axis that the shaft extends along. A coupler may couple the elongate shafts to each other such that the elongate shaft including the implant may slide relative to the steerable elongate shaft, with the axes of the shafts offset from each other.

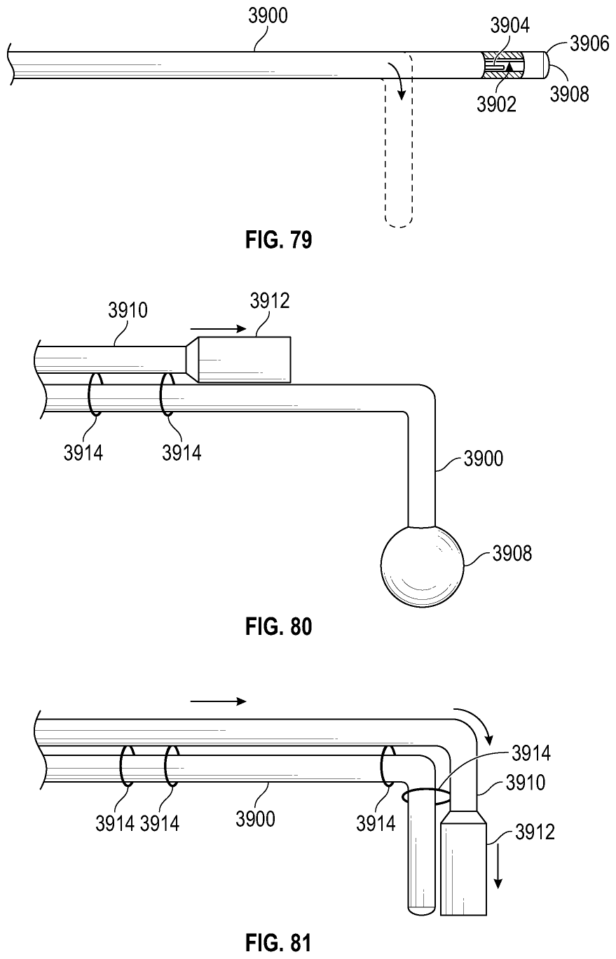

FIG. 79, for example, illustrates an embodiment of a steerable elongate shaft 3900 of a delivery system. The elongate shaft 3900 may be steerable utilizing mechanisms disclosed herein, for example via the use of pull tethers, or may be steerable via another mechanism. The elongate shaft 3900 may be configured to form one or more bends in the elongate shaft 3900 and may be configured to be steerable and bend in at least one plane, or at least two planes as disclosed herein. The steerable elongate shaft 3900 may be configured to be inserted into the patient's body and moved to the desired implantation site for the implant.

The elongate shaft 3900 may be configured to be steerable and may be constructed in a similar manner as a rail assembly 20 as disclosed herein. For example, pull tethers may be utilized to control the deflection of the elongate shaft 3900 in one or more planes, or at least two planes as desired. Unlike the rail assembly 20 disclosed herein, the elongate shaft 3900 may be inserted into the patient's body without the implant and that capsule retaining the implant.

The embodiments disclosed herein may be utilized in a method including deploying an elongate shaft to a location within a patient's body, the elongate shaft including a proximal end and a distal end and an implant retention area retaining an implant for implantation within a patient's body. The method may include deflecting the distal end of the elongate shaft utilizing a pull tether to a portion of the elongate shaft at or distal the implant retention area. The pull tether 3700 and configuration of the pull tether 3700 may be utilized solely or with any of the other apparatuses, systems, or methods disclosed herein.

The elongate shaft 3900 may include an interior lumen 3902 that may be configured to retain components for the delivery system, such as an imaging sensor 3904, such as an ultrasound intra-cardiac echo (ICE) sensor, or other form of imaging sensor as desired that is configured to be positioned within the interior lumen 3902. A distal end 3906 of the shaft 3900 may include an inflatable body 3908 that may be configured to inflate to secure the distal end 3906 of the shaft 3900 in a position and/or determine if the pathway formed by the shaft 3900 is clear of obstructions (e.g., the distal end 3906 does not pass between chordae of a patient's heart).

The elongate shaft 3900 may be introduced first into a patient's body and steered to the desired implantation site.

Referring to FIG. 80, the elongate shaft 3910 may comprise a shaft configured to retain the implant for deployment. The shaft 3910, for example, may include a capsule 3912 at a distal end that retains the implant. The capsule 3912 may surround the implant retention area and retain the implant therein, as disclosed herein. The shaft 3910 may include a deployment mechanism for deploying the implant from the capsule 3912 as disclosed herein. For example, the capsule 3912 may be retracted to expose and deploy the implant. The shaft 3910 may be flexible, and passively flexible, to allow the shaft 3910 to follow the path that is formed by the steerable elongate shaft 3900.

A coupler 3914 may couple the elongate shaft 3910 to the steerable elongate shaft 3900 The coupler 3914 may have a variety of forms and may include a loop as shown in FIG. 80, or may comprise one or more of a magnet, a hook, a loop, or a joint between the shafts 3910, 3900. The coupler 3914 may be configured to allow the elongate shaft 3910 to slide relative to the shaft 3900. In embodiments, the coupling may occur within the patient's body, or may occur outside the patient's body as desired.

The elongate shafts 3900, 3910 may be coupled together such that the respective axes of the shafts 3900, 3910 are offset from each other. The coupler 3914 may be configured to couple the shafts 3900, 3910 to each other such that the shafts 3900, 3910 may slide with the respective axes of the shafts 3900, 3910 parallel with each other, and with the outer surfaces of the shafts 3900, 3910 adjacent to each other. As such, the deployment mechanism (utilized with elongate shaft 3910) may be separated into a separate shaft than the steering mechanism (utilized with elongate shaft 3900). The complexity of each shaft accordingly may be reduced from an embodiment shown in FIG. 1. Further, the capsule 3912 in such an embodiment is not positioned distal of a bend portion, which may enhance the maneuverability of the capsule 3912 in an embodiment as shown in FIG. 80.

In embodiments, the coupler 3914 may be configured to couple the shafts 3900, 3910 together in a defined orientation. For example, a joint such as a dovetail joint may be provided on either shaft 3900, 3910 such that the shafts 3900, 3910 may only couple in a defined orientation. As such, a circumferential position of the 3900 relative to the shaft 3910 may be defined. Such a feature may be beneficial in an embodiment in which an asymmetric implant is deployed. Such an implant may be deployed with the steerable shaft 3900 in a defined position that may aid the deployment of the asymmetric implant. In embodiments, the coupler 3914 may be configured such that the coupling may be rotated to orient the implant to optimize the position of the implant for deployment. In other embodiments, other forms of coupling may be utilized.

Referring back to FIG. 79, in operation, the steerable elongate shaft 3900 may be advanced to the desired location for implantation within the patient's body. The steerable elongate shaft 3900 may be bent in a desired configuration, and may retain that configuration within the patient's body. The distal end 3906 of the elongate shaft 3900 for example, may be steered to the mitral valve, or other valve as desired. In embodiments, as shown in FIG. 80, the inflatable body 3908 may be inflated to secure the distal end 3906 of the shaft 3900 in a position and/or determine if the pathway formed by the shaft 3900 is clear of obstructions.

The steerable elongate shaft 3900 in position within the patient's body may serve as a rail that the elongate shaft 3910 having the implant is slid along. The elongate shaft 3910 may be passed through a separate entry point in the patient's body, for example, a separate leg or separate venous body of the patient's body. In embodiments, the coupler 3914 may couple the shafts 3900, 3910 together within the patient's body such that the shaft 3910 may slide along the steerable shaft 3900. The shaft 3910 may be advanced through the patient's body along the steerable shaft 3900 to the desired implantation site as shown in FIG. 80.

The shaft 3910 may be configured to deflect about any bend in the steerable shaft 3900 as shown in FIG. 81. The deflection of the shaft 3910 may be passive if desired. The shaft 3910 may then be utilized to deploy the implant from the capsule 3912. A deployment mechanism as disclosed herein may be operated and the capsule 3912 may be retracted to deploy the implant. Following deployment, the shaft 3910 may be withdrawn, and then the shaft 3900 may be subsequently withdrawn. The configuration of the shafts 3900, 3910 may be utilized solely or with any of the other apparatuses, systems, or methods disclosed herein.

From the foregoing description, it will be appreciated that an inventive product and approaches for implant delivery systems are disclosed. While several components, techniques and aspects have been described with a certain degree of particularity, it is manifest that many changes can be made in the specific designs, constructions and methodology herein above described without departing from the spirit and scope of this disclosure. The disclosure is not limited to the system and apparatuses disclosed herein, but also the methods of utilizing such systems and apparatuses.

Certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as any subcombination or variation of any subcombination.

Moreover, while methods may be depicted in the drawings or described in the specification in a particular order, such methods need not be performed in the particular order shown or in sequential order, and that all methods need not be performed, to achieve desirable results. Other methods that are not depicted or described can be incorporated in the example methods and processes. For example, one or more additional methods can be performed before, after, simultaneously, or between any of the described methods. Further, the methods may be rearranged or reordered in other implementations. Also, the separation of various system components in the implementations described above should not be understood as requiring such separation in all implementations, and it should be understood that the described components and systems can generally be integrated together in a single product or packaged into multiple products. Additionally, other implementations are within the scope of this disclosure.

Conditional language, such as “can,” “could,” “might,” or “may,” unless specifically stated otherwise, or otherwise understood within the context as used, is generally intended to convey that certain embodiments include or do not include, certain features, elements, and/or steps. Thus, such conditional language is not generally intended to imply that features, elements, and/or steps are in any way required for one or more embodiments.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than or equal to 10% of, within less than or equal to 5% of, within less than or equal to 1% of, within less than or equal to 0.1% of, and within less than or equal to 0.01% of the stated amount. If the stated amount is 0 (e.g., none, having no), the above recited ranges can be specific ranges, and not within a particular % of the value. For example, within less than or equal to 10 wt./vol. % of, within less than or equal to 5 wt./vol. % of, within less than or equal to 1 wt./vol. % of, within less than or equal to 0.1 wt./vol. % of, and within less than or equal to 0.01 wt./vol. % of the stated amount.

Some embodiments have been described in connection with the accompanying drawings. The figures are drawn to scale, but such scale should not be limiting, since dimensions and proportions other than what are shown are contemplated and are within the scope of the disclosed inventions. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various embodiments can be used in all other embodiments set forth herein. Additionally, it will be recognized that any methods described herein may be practiced using any device suitable for performing the recited steps.

While a number of embodiments and variations thereof have been described in detail, other modifications and methods of using the same will be apparent to those of skill in the art. Accordingly, it should be understood that various applications, modifications, materials, and substitutions can be made of equivalents without departing from the unique and inventive disclosure herein or the scope of the claims.

What is claimed is:

1. A delivery system for delivering an implant to a location within a patient's body, the delivery system comprising:
a capsule, located at a distal portion of the delivery system, configured to house the implant, and having a distal opening;
a retention member, wherein:
the delivery system has a delivery state in which the retention member retains the implant in the capsule; and
the capsule is proximally slidable with respect to the retention member to transition the system from the delivery state to a deployment state in which the implant is deployed out of the distal opening; and
at the distal portion, a stop, shaped and positioned to define a stopping point, partway to the deployment state, at which the stop impedes the proximal sliding of the capsule.

2. The delivery system according to claim 1, wherein:
the delivery system further includes the implant;
the implant is retained within the capsule by the retention member; and
the stop is positioned such that sliding the capsule proximally toward the stop partially exposes the implant.

3. The delivery system according to claim 2, wherein the capsule is configured to allow for full exposure of the implant upon the capsule overcoming the stop and sliding the capsule proximally relative to the retention member such that the implant is exposed.

4. The delivery system according to claim 3, wherein the implant is configured to be fully exposed when capsule slides past the retention member.

5. A delivery system for delivering an implant to a location within a patient's body, the delivery system comprising:
a shaft having a retention member that is configured to retain the implant, the retention member positioned at a distal end portion of the shaft;
a sheath, slidably mounted on the shaft, and configured to surround the retention member; and
a stop, positioned at the distal end portion of the shaft proximal to the retention member, and configured to define a stopping point at which the stop impedes proximal sliding of the sheath past the stop.

6. The delivery system according to claim 5, wherein the stop includes a protrusion extending radially outward from the shaft.

7. The delivery system according to claim 6, wherein the protrusion is configured such that applying a predetermined proximal force on the sheath, while the sheath is against the stop, causes the sheath to overcome the protrusion and allows the sheath to move proximally past the stop.

8. The delivery system according to claim 7, wherein the protrusion is configured to elastically deform when the predetermined proximal force is applied to the sheath against the stop such that the sheath overcomes the protrusion.

9. The delivery system according to claim 7, wherein the sheath is configured to elastically deform when the predetermined proximal force is applied to the sheath against the stop such that the sheath overcomes the protrusion.

10. The delivery system according to claim 5, wherein the stop includes first threads and the sheath includes second threads that are complementary to the first threads.

11. The delivery system according to claim 10, wherein threading the first threads through the second threads allows the sheath to slide proximally past the stop.

12. The delivery system according to claim 5, wherein the stop includes a keyed shape and the sheath includes a complementary shape to the keyed shape.

13. The delivery system according to claim 12, wherein the sheath is configured to rotate relative to the stop to accommodate the keyed shape such that the sheath is allowed to move proximally past the stop.

14. The delivery system according to claim 5, wherein the sheath includes a proximal end portion including:
a shaft portion at a proximal end of the proximal end portion, the shaft portion having a smaller diameter than the retention member; and
a seat portion configured to:
receive the retention member; and
prevent the proximal end portion from sliding past the retention member.

15. The delivery system according to claim 14, wherein the sheath is configured to surround the implant while the retention member is seating against the seat portion.

16. The delivery system according to claim 5, wherein the stop is slidable relative to the shaft.

17. The delivery system according to claim 5, wherein the stop is fixed relative to the shaft.

18. The delivery system according to claim 5, wherein the stop is integral with the shaft.

19. A delivery system for delivering an implant to a location within a patient's body, the delivery system comprising:
an elongate shaft having a proximal end and a distal end, and including:
an implant retention area configured to retain the implant;
a sheath having an interior lumen and a capsule configured to surround the implant retention area;
an interior shaft positioned within the interior lumen; and
a stop positioned on the interior shaft and configured to impede proximal movement of the capsule relative to the interior shaft.

20. The delivery system according to claim 19, wherein the elongate shaft includes an inner shaft, and the interior shaft comprises a middle shaft positioned between the inner shaft and the sheath.
